(19) United States
(12) Patent Application Publication
(10) Pub. No.: US 2020/0031895 A1
(43) Pub. Date: Jan. 30, 2020

(54) STABILIZED PROTEOLYTICALLY ACTIVATED GROWTH DIFFERENTIATION FACTOR 11

(71) Applicant: Biogen MA Inc., Cambridge, MA (US)

(72) Inventors: R. Blake Pepinsky, Arlington, MA (US); Andreas Lehmann, Belmont, MA (US); Dingyi Wen, Waltham, MA (US)

(21) Appl. No.: 16/468,347

(22) PCT Filed: Dec. 15, 2017
(86) PCT No.: PCT/US2017/066705

§ 371 Date: Jun. 11, 2019

Related U.S. Application Data
(60) Provisional application No. 62/435,493, filed on Dec. 16, 2016.

(51) Int. Cl. C07K 14/51; C12P 21/02

(52) U.S. Cl. CPC C07K 14/51; A61K 38/00; C12P 21/02

(57) ABSTRACT

Methods of activating GDF11 proteins in vitro as well as formulations of mature GDF11 polypeptides with enhanced solubility at neutral pH are provided.

### CROSS-REFERENCE TO RELATED APPLICATIONS

This application claims priority to U.S. Provisional Appl. No. 62/435,493, filed Dec. 16, 2016, the contents of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Dec. 14, 2016, is named 13751-0263WO1_SL.txt and is 73,561 bytes in size.

### BACKGROUND

Growth differentiation factor 11 (GDF11, also known as bone morphogenetic protein 11) is a member of the transforming growth-β (TGF-β) family of proteins that play diverse roles in regulation of embryonic patterning and morphogenesis, cell proliferation and differentiation, adhesion, immune responses, cell growth arrest and tissue or organ regeneration and maintenance (Massague, Nature Rev. Mol. Cell Biol., 1:169-178 (2000); Chang et al., Endocr Rev., 23:787-823 (2002); Heldin et al., Curr Opin Cell Biol., 21:166-76 (2009); Wakefield and Hill, Nat Rev Cancer, 13:328-41 (2013)). This superfamily contains over 30 members, including TGFβs, activins, inhibins, bone morphogenetic proteins (BMPs), growth and differentiation factors (GDFs), glial cell line-derived neurotrophic factor family of ligands (GFLs), nodal and anti-Mullerian hormone (AMH) (Hinck, FEBS Lett., 586:1860-70 (2012); Weiss and Attisano, Wiley Interdiscip Rev Dev Biol., 2:47-63 (2013)). All of the proteins are expressed as large precursor proteins that undergo proteolytic processing to form non-covalently associated N-terminal pro- and C-terminal mature domains. Processing of the precursors at mono and/or dibasic cleavage sites at the junction of the pro and mature domains can result in the formation of an active complex or a latent complex that requires further processing/activation steps. Most members of the family form active complexes. However, like TGF-β1-3 and myostatin, processing of GDF11 forms a latent complex. Subsequent cleavage at a BMP1 site within the prodomain has been shown to lead to activation of GDF11 (Ge et al., Mol Cell Biol., 25:5846-5858 (2005)). The mature domain of GDF11 is 109 amino acids in length and is not glycosylated. It consists of amino acids 299-407, containing 9 cysteines that form 4 intramolecular disulfides and one interchain disulfide stabilizing the homodimer structure.

GDF11 contributes to early mesoderm development and anterior-posterior patterning of the axonal skeleton. In addition, GDF11 has been identified to have roles in inhibiting neurogenesis in olfactory epithelium, preventing NGF-induced neurite outgrowth in PC12 cells, and in promoting vascular remodeling and neurogenesis in animals treated with GDF11 (Ge et al., (supra); Katsimpardi et al., Science, 344:630-634 (2014)). In view of the roles of GDF11 it is highly desirable to obtain pharmaceutical formulations for use in vivo.

The development of formulations suitable for in vivo administration is a critical part of drug development, requiring individualized optimization for each product (Wang et al, J. Pharm. Sci., 96:1-26 (2007); Uchiyama, Biochim Biophys Acta, 1844:2041-2052 (2014)). Ideal formulations enhance product stability, minimize against degradative pathways such as chemical modification, aggregation and precipitation, improve solubility, improve bioavailability, and can minimize injection site reactions or immunogenicity. The poor solubility of the mature domains of most TGF-β family members at neutral pH is a common occurrence that is routinely addressed by using acidic formulations. Acid formulations carry liabilities in that they promote reversible or irreversible denaturation, chemical degradation, increase surface charge, and frequently lead to precipitation/deposition at sites of injection due to the rapid increase in pH upon delivery. Thus, there is an unmet need for GDF11 compositions that are soluble at neutral pH.

### SUMMARY

This disclosure is based in part on the surprising finding that certain prodomain peptides of GDF11 enhance the solubility of the mature domain of GDF11 at neutral pH without inactivating the activity of the mature domain.

In a first aspect, the disclosure features an isolated multimeric GDF11 protein comprising a first polypeptide, a second polypeptide, a third polypeptide, and a fourth polypeptide. In certain instances, the isolated multimeric GDF11 protein comprises a first polypeptide, a second polypeptide, and a fourth polypeptide. In other instances, the multimeric GDF11 protein comprises a second polypeptide, a third polypeptide, and a fourth polypeptide. The first polypeptide is non-covalently associated with the second polypeptide and the third polypeptide is non-covalently associated with the fourth polypeptide. The second polypeptide is linked to the fourth polypeptide by a disulfide bond. The first polypeptide and the third polypeptide each comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 60-112 of SEQ ID NO:1, and the second polypeptide and the fourth polypeptide each comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 296-407 or 299-407 of human GDF11 (SEQ ID NO:1). In some instances, the first polypeptide and the third polypeptide can contain one to ten (e.g., 1, 2, 3, 4, 5, 6, 7, 8, 9, or 10) substitutions, insertions, and/or deletions within amino acids 60-112 of SEQ ID NO:1. In some instances, the second polypeptide and the fourth polypeptide can contain one to ten (e.g., 1, 2, 3, 4, 5, 6, 7, 8, 9, or 10) substitutions, insertions, and/or deletions within amino acids 296-407 or 299-407 of human GDF11 (SEQ ID NO:1). In some instances, the substitutions are conservative amino acid substitutions. In certain instances, the second polypeptide and the fourth polypeptide can contain one, two, or three deletions at the N- and/or C-terminus of amino acids 296-407 or 299-407 of human GDF11. The multimeric protein can induce SMAD 2/3 phosphorylation in a Kinase Induced Receptor Activation Assay (KIRA).

In a second aspect, the disclosure provides an isolated multimeric GDF11 protein comprising at least two or more of a first polypeptide, a second polypeptide, a third polypeptide, and a fourth polypeptide. In certain instances, the isolated multimeric GDF11 protein comprises a first polypeptide, a second polypeptide, and a fourth polypeptide. In other instances, the isolated multimeric GDF11 protein comprises a second polypeptide, a third polypeptide, and a fourth polypeptide. The first polypeptide is non-covalently associated with the second polypeptide and the third polypeptide is non-covalently associated with the fourth polypeptide. The second polypeptide is linked to the fourth polypeptide by a disulfide bond. The first polypeptide and the third polypeptide each comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 60-112 of SEQ ID NO:1, and the second polypeptide and the fourth polypeptide each comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 296-407 or 299-407 of human GDF11 (SEQ ID NO:1). In some instances, the first polypeptide and the third polypeptide can contain one to ten (e.g., 1, 2, 3, 4, 5, 6, 7, 8, 9, or 10) substitutions, insertions, and/or deletions within amino acids 60-112 of SEQ ID NO:1. In some instances, the second polypeptide and the fourth polypeptide can contain one to ten (e.g., 1, 2, 3, 4, 5, 6, 7, 8, 9, or 10) substitutions, insertions, and/or deletions within amino acids 296-407 or 299-407 of human GDF11 (SEQ ID NO:1). In some instances, the substitutions are conservative amino acid substitutions. In certain instances, the second polypeptide and the fourth polypeptide can contain one, two, or three deletions at the N- and/or C-terminus of amino acids 299-407 of human GDF11. The multimeric GDF11 protein can induce SMAD 2/3 phosphorylation in a KIRA.

These embodiments apply to the first and second aspects. In some embodiments, the first polypeptide and the third polypeptide are each at least 95% identical to amino acids 60-112 of SEQ ID NO:1. In other embodiments, the first polypeptide and the third polypeptide each consist of an amino acid sequence selected from the group consisting of amino acids 60-112, 60-114, and 60-117 of SEQ ID NO:1. In certain embodiments, the first and third polypeptides consist of amino acids 60-112 and 60-114, respectively, or vice versa. In other embodiments, the first and third polypeptides consist of amino acids 60-112 and 60-117 of SEQ ID NO:1, or vice versa. In yet other embodiments, the first and third polypeptides consist of amino acids 60-114 and 60-117 of SEQ ID NO:1, or vice versa. In certain embodiments, the first and third polypeptides each consist of amino acids 60-112 of SEQ ID NO:1. In other embodiments, the first and third polypeptides each consist of amino acids 60-114 of SEQ ID NO:1. In yet other embodiments, the first and third polypeptides each consist of amino acids 60-117 of SEQ ID NO:1. In certain embodiments, the second polypeptide and the fourth polypeptide are each at least 95% identical to amino acids 296-407 or 299-407 of SEQ ID NO:1. In other embodiments, the second polypeptide and the fourth polypeptide are each identical to amino acids 296-407 or 299-407 of SEQ ID NO:1. In a particular embodiment, the first polypeptide and the third polypeptide each consist of amino acids 60-112, 60-114, or 60-117 of SEQ ID NO:1 and the second polypeptide and the fourth polypeptide each consist of amino acids 296-407 or 299-407 of SEQ ID NO:1. In certain embodiments, the asparagine at the position corresponding to position 94 of SEQ ID NO:1 is glycosylated in each of the first polypeptide and the third polypeptide. In certain embodiments, one or more of the polypeptides is linked to a half-life extending moiety. In certain instances, the half-life extending moiety is PEG, XTEN, BSA, or an Fc moiety. In certain embodiments, one or more of the polypeptides is linked to an agent that can traverse the blood brain barrier (e.g., FC5, FC5-Fc, anti-transferrin receptor antibody; insulin receptor antibody, insulin-like growth factor-1 receptor antibody, see e.g., Jones and Shusta, Blood Brain Transport of Therapeutics via Receptor Mediation, Pharm Res. 24:1759-1771 (2007), incorporated by reference in its entirety herein). In some embodiments, the multimeric protein is in a pharmaceutical composition comprising a pharmaceutically acceptable carrier. In some instances, the pH of the pharmaceutical composition is in the range of 5.0 to 6.5. In other instances, the pH of the pharmaceutical composition is about 5.5. In certain instances, the protein remains soluble at pH 7.0.

In a third aspect, the disclosure features an isolated protein comprising, in order, a first amino acid sequence and a second amino acid sequence linked directly via a linker (e.g., a peptide linker of 5 to 100 amino acids in length). The first amino acid sequence is 52 to 65 amino acids in length and comprises an amino acid sequence that is at least 70%, at least 75%, at least 80%, at least 85%, at least 90%, at least 95%, at least 97% identical, or 100% identical to amino acids 60 to 114 of SEQ ID NO:1, or amino acids 71-123 of SEQ ID NO:1. The second amino acid sequence comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 296-407 or 299-407 of SEQ ID NO:1. In some instances, the first amino acid sequence can contain one to ten (e.g., 1, 2, 3, 4, 5, 6, 7, 8, 9, or 10) substitutions, insertions, and/or deletions within amino acids 60-114 of SEQ ID NO:1. In some instances, the second amino acid sequence can contain one to ten (e.g., 1, 2, 3, 4, 5, 6, 7, 8, 9, or 10) substitutions, insertions, and/or deletions within amino acids 296-407 or 299-407 of human GDF11 (SEQ ID NO:1). In some instances, the substitutions are conservative amino acid substitutions. The protein when activated by dimerization or by dimerization and proteolytic cleavage (e.g., with an endoproteinase) can induce SMAD 2/3 phosphorylation in a KIRA.

In certain embodiments, the first amino acid sequence is 52 to 62 amino acids in length (e.g., 52, 53, 54, 55, 56, 57, 58, 59, 60, 61, or 62 amino acids in length). In other embodiments, the first amino acid sequence is 52 to 55 amino acids in length. In certain instances, the first amino acid sequence consists of amino acids 71-123, 60-122, or 60-114 of SEQ ID NO:1. In some embodiments, the second amino acid sequence is at least 95% identical to amino acids 296-407 or 299-407 of SEQ ID NO:1. In other embodiments, the second amino acid sequence consists of amino acids 296-407 or 299-407 of SEQ ID NO:1. In certain embodiments, the peptide linker is 10 to 150, 10 to 144, 10 to 100, 10 to 50, 10 to 40, 10 to 30, 10 to 20, or 10 to 15 amino acids in length. In a specific embodiment, the peptide linker is 15 amino acids in length. In another specific embodiment, the peptide linker is 20 amino acids in length. In yet another specific embodiment, the peptide linker is 144 amino acids in length. In a specific embodiment, the peptide linker comprises or consists of G₄5 (SEQ ID NO: 4). In certain embodiment the serine in SEQ ID NO:4 can be replaced with another amino acid. In another embodiment, the peptide linker lacks the GSG amino acid sequence. In another embodiment, the peptide linker is an XTEN (e.g., AE 144). In certain embodiments, the protein comprises amino acids 35-211 of SEQ ID NO:6; amino acids 36-217 of SEQ ID NO:14; amino acids 36-227 of SEQ ID NO:5; or amino acids 36-219 of SEQ ID NO:9. In certain embodiments, the protein is linked to a half-life extending moiety. In certain instances, the half-life extending moiety is PEG, XTEN, HSA, or an Fc moiety. In certain embodiments, the protein is linked to an agent that can traverse the blood brain barrier (e.g., FC5, FC5-Fc, anti-transferrin receptor antibody; insulin receptor antibody, insulin-like growth factor-1 receptor antibody). In some embodiments, the protein is in a pharmaceutical composition comprising a pharmaceutically acceptable carrier. In some instances, the pH of the pharmaceutical composition is in the range of 5.0 to 6.5. In other instances, the pH of the pharmaceutical composition is about 5.5. In certain instances, the protein remains soluble at pH 7.0. In certain embodiments, provided is a nucleic acid encoding the protein of this aspect. In some embodiments, expression vectors comprising the nucleic acid encoding the protein of this aspect are provided. In some embodiments, the disclosure encompasses isolated host cells comprising the nucleic acid or expression vector described above. Also provided are methods of making the protein of this aspect. The method involves culturing the host cell in a culture medium under conditions in which the protein is produced by the host cell (e.g., secreted into the culture medium). The method optionally involves isolating the protein. The isolated protein can be activated. In some embodiments, the protein is subjected to a disulfide reducing agent to create a first composition. The first composition is divided into a second and a third composition. The second composition is treated with or exposed to a cysteine activating agent to create a fourth composition. The fourth composition is combined with the third composition to create a fifth composition. In some instances, the fifth composition is active. In certain cases, the fifth composition is treated with a protease that cleaves at the BMP1 site of the protein, thereby making an activated protein. In certain embodiments, the disulfide reducing agent is DTT. In certain embodiments, the cysteine activating agent is aldrithiol. In some embodiments, the protease that cleaves at the BMP1 site of the protein is endoproteinase AspN. In certain instances, the method further involves formulating the fourth composition (if active) or fifth composition at a pH of 5.0 to 6.5. In a specific embodiment, the pH is 5.5. In some embodiments, the protein is produced by a mammalian cell (e.g., a CHO, COS, 293, NIH3T3 cell). In some embodiments, the protein is produced by a fungal cell (e.g., Aspergillus). In other embodiments, the protein is produced by an algal cell.

In a fourth aspect, the disclosure provides an isolated multimeric GDF11 protein comprising a first polypeptide and a second polypeptide. The first polypeptide comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 296-407 or 299-407 of human GDF11 (SEQ ID NO:1). The second polypeptide comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to any one of: SPRELRLESIKSQILSKLRLKEAPNISREVVKQLLPKAPPLQQIL (SEQ ID NO:21); SPRELRLESIKSQILSKLRLKEAPNISREVVKQLLPKAPP (SEQ ID NO:28); SPRELRLESIKSQILSKLRLKEAPNIS (SEQ ID NO:29); DGCPVCVWRQHSRELRLESIKSQILSKLRLKEAPNIS (SEQ ID NO:30); DGCPVCVWRQHSRELRLESIKSQILSKLRLKG (SEQ ID NO:31); or SPRELRLESIKSQILSKLRLKG (SEQ ID NO:32). In certain embodiments, the first and/or second polypeptide can have one to ten amino acid substitutions, deletions, and/or insertions within amino acids 296-407 or 299-407 of SEQ ID NO:1 or within SEQ ID NOs.: 21, or 28-32. The multimeric protein can induce SMAD 2/3 phosphorylation in a KIRA.

In certain embodiments, the first polypeptide consists of amino acids 296-407 or 299-407 of SEQ ID NO:1. In other embodiments, the second polypeptide consists of the amino acid sequence of SEQ ID NO:21. In certain embodiments, the first and/or second polypeptide is linked to a half-life extending moiety. In certain instances, the half-life extending moiety is PEG, XTEN, HSA, or an Fc moiety. In certain embodiments, the first and/or second polypeptide is linked to an agent that can traverse the blood brain barrier (e.g., FC5, FC5-Fc, anti-transferrin receptor antibody; insulin receptor antibody, insulin-like growth factor-1 receptor antibody). In some embodiments, the protein is in a pharmaceutical composition comprising a pharmaceutically acceptable carrier. In some instances, the pH of the pharmaceutical composition is in the range of 5.0 to 6.5. In other instances, the pH of the pharmaceutical composition is about 5.5. In certain instances, the protein remains soluble at pH 7.0.

In a fifth aspect, provided is a pharmaceutical composition comprising a pharmaceutically acceptable carrier and a population of dimeric GDF11 proteins. The dimeric GDF11 proteins in the population comprise two GDF11 monomers each of which consists of an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 296-407 or 299-407 of human GDF11 (SEQ ID NO:1). At least 80% of the dimeric GDF11 proteins in the population comprise a polypeptide non-covalently associated with each GDF11 monomer. The polypeptide comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 60-112 of SEQ ID NO:1. The GDF11 proteins can induce SMAD 2/3 phosphorylation in a KIRA.

In some embodiments, at least 85% of the dimeric GDF11 proteins in the population comprise the polypeptide non-covalently associated with each GDF11 monomer. In some embodiments, at least 90% of the dimeric GDF11 proteins in the population comprise the polypeptide non-covalently associated with each GDF11 monomer. In some embodiments, at least 95% of the dimeric GDF11 proteins in the population comprise the polypeptide non-covalently associated with each GDF11 monomer. In some embodiments, at least 97% of the dimeric GDF11 proteins in the population comprise the polypeptide non-covalently associated with each GDF11 monomer. The polypeptide non-covalently associated with each GDF11 mature domain monomer may be the same GDF11 propeptide polypeptide or different GDF11 propeptide polypeptides (e.g., 60-112 and 60-114; 60-112 and 60-117; or 60-114 and 60-117). In certain embodiments, the polypeptide consists of amino acids 60-112, 60-114, or 60-117 of SEQ ID NO:1 and each GDF11 monomer consists of amino acids 296-407 or 299-407 of SEQ ID NO:1. In certain embodiments, the asparagine at position 94 of SEQ ID NO:1 is glycosylated in the polypeptide.

In a sixth aspect, featured is a pharmaceutical composition comprising a pharmaceutically acceptable carrier and a population of dimeric GDF11 proteins. The dimeric GDF11 proteins in the population comprise two GDF11 monomers each of which consists of an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 296-407 or 299-407 of human GDF11 (SEQ ID NO:1). At least 80% of the dimeric GDF11 proteins in the population comprise a polypeptide non-covalently associated with each GDF11 monomer. The polypeptide comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to: SPRELRLESIKSQILSKLRLKEAPNISREVVKQLLPKAPPLQQIL (SEQ ID NO:21); SPRELRLESIKSQILSKLRLKEAPNISREVVKQLLPKAPP (SEQ ID NO:28); SPRELRLESIKSQILSKLRLKEAPNIS (SEQ ID NO:29); DGCPVCVWRQHSRELRLESIKSQILSKLRLKEAPNIS (SEQ ID NO:30); DGCPVCVWRQHSRELRLESIKSQILSKLRLKG (SEQ ID NO:31); or SPRELRLESIKSQILSKLRLKG (SEQ ID NO:32). The dimeric GDF11 protein non-covalently associated with the polypeptide can induce SMAD 2/3 phosphorylation in a KIRA.

In some embodiments, at least 85% of the dimeric GDF11 proteins in the population comprise the polypeptide non-covalently associated with each GDF11 monomer. In some embodiments, at least 90% of the dimeric GDF11 proteins in the population comprise the polypeptide non-covalently associated with each GDF11 monomer. In some embodiments, at least 95% of the dimeric GDF11 proteins in the population comprise the polypeptide non-covalently associated with each GDF11 monomer. In some embodiments, at least 97% of the dimeric GDF11 proteins in the population comprise the polypeptide non-covalently associated with each GDF11 monomer. In certain embodiments, the polypeptide comprises or consists of the amino acid sequence: SPRELRLESIKSQILSKLRLKEAPNISREVVKQLLPKAPPLQQIL (SEQ ID NO:21). In certain embodiments, the asparagine at position 94 of SEQ ID NO:1 is glycosylated in the polypeptide.

In a seventh aspect, the disclosure provides a method of producing an activated human GDF11 protein. The method involves contacting a GDF11 protein with a first protease that cleaves at a BMP1 site of the GDF11 protein; and then contacting the protein with a second protease (e.g., furin, plasmin, or trypsin).

In certain embodiments, the GDF11 protein is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to a full length human GDF11 protein. In some embodiments, the GDF11 protein is a full length human GDF11 protein. In certain embodiments, the human GDF11 protein is obtained from a mammalian cell (e.g., CHO, COS, 293, NIH3T3). In certain embodiments, the human GDF11 protein is obtained from a yeast cell (e.g., Pichia pastoris). In certain embodiments, the human GDF11 protein is obtained from a microbial cell (e.g., E. coli). In certain embodiments, the human GDF11 protein is obtained from an algal cell. In certain embodiments, the human GDF11 protein is obtained from a fungal cell. In some embodiments, the protease that cleaves at the BMP1 site of the GDF11 protein is endoproteinase AspN. In some embodiments, the second protease is furin. In some embodiments, the second protease is plasmin. In some embodiments, the second protease is trypsin. In certain instances, the method further involves formulating the activated human GDF11 protein as a pharmaceutical composition. In some embodiments, the pharmaceutical composition is a sterile composition and has a pH in the range of 5.0 to 6.5. In other embodiments, the pharmaceutical composition is a sterile composition and has a pH of about 5.5.

In an eighth aspect, this disclosure features a method of preparing a GDF11 protein formulation, the method comprising combining a first GDF11 polypeptide and a second GDF11 polypeptide. The first GDF11 polypeptide comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to: SPRELRLESIKSQILSKLRLKEAPNISREVVKQLLPKAPPLQQIL (SEQ ID NO:21); SPRELRLESIKSQILSKLRLKEAPNISREVVKQLLPKAPP (SEQ ID NO:28); SPRELRLESIKSQILSKLRLKEAPNIS (SEQ ID NO:29); DGCPVCVWRQHSRELRLESIKSQILSKLRLKEAPNIS (SEQ ID NO:30); DGCPVCVWRQHSRELRLESIKSQILSKLRLKG (SEQ ID NO:31); or SPRELRLESIKSQILSKLRLKG (SEQ ID NO:32). The second GDF11 polypeptide comprises an amino acid sequence that is at least 70%, at least 75%, at least 80%, at least 85%, at least 90%, at least 91%, at least 92%, at least 93%, at least 94%, at least 95%, at least 96%, at least 97% identical, at least 98%, at least 99%, or 100% identical to amino acids 299-407 of human GDF11 (SEQ ID NO:1). In some embodiments, the first polypeptide comprises or consists of SPRELRLESIKSQILSKLRLKEAPNISREVVKQLLPKAPPLQQIL (SEQ ID NO:21). In some embodiments, the second polypeptide consists of amino acids 299-407 of SEQ ID NO:1. In some embodiments the first GDF11 polypeptide is 45, 44, 43, 42, 41, 40, 39, 38, 37, 36, 35, 34, 33, 32, 31, 30, 29, 28, 27, 26, or 25 amino acids in length. In certain embodiments, the first and/or second polypeptide is/are linked to a half-life extending moiety (e.g., PEG, XTEN, HSA, Fc moiety). In certain embodiments, the first and/or second polypeptide is linked to an agent that can traverse the blood brain barrier (e.g., FC5, FC5-Fc, anti-transferrin receptor antibody; insulin receptor antibody, insulin-like growth factor-1 receptor antibody). In certain embodiments, the method further comprises adjusting the pH of the formulation to between 5.0 and 6.5. In a specific embodiment, the pH of the formulation is about 5.5. In certain embodiments, the formulation is a sterile formulation.

In a ninth aspect, the disclosure features a method of treating a neurodegenerative disease in a human subject in need thereof. In certain embodiments, the neurodegenerative disease is a disease of the central nervous system. In other embodiments, the neurodegenerative disease is a disease of the peripheral nervous system. The method involves administering to the human subject a therapeutically effective amount of a protein(s) or a pharmaceutical composition described herein.

In some embodiments, the neurodegenerative disease is Alzheimer's disease, Parkinson's disease, Huntington's disease, amyotrophic lateral sclerosis, frontotemporal Dementia, Lewy Body Dementia, Mild Cognitive Impairment, Posterior Cortical Atrophy, Primary Progressive Aphasia, Progressive Supranuclear Palsy, or Vascular Dementia. In a particular embodiment, the neurodegenerative disease is Alzheimer's disease. In another particular embodiment, the neurodegenerative disease is Parkinson's disease. In yet another particular embodiment, the neurodegenerative disease is Lewy Body Dementia.

In some embodiments, the neurodegenerative disease is spinal muscular atrophy (SMA), myasthenia gravis, Isaacs syndrome, Stiff-Person syndrome, Guillian-Barre syndrome, chronic inflammatory demyelinating polyneuropathy, amyotrophic lateral sclerosis, peripheral neuropathy, or thoracic outlet compression syndrome.

In another aspect, the disclosure features a polypeptide comprising or consisting of the amino acid sequences set forth in any one of SEQ ID NO:21 or SEQ ID NO: 28-32. In certain instances, these polypeptides have at least two (e.g., 2, 3, 4, 5, 6, 7, 8) amino acid substitutions. For example, the amino acids may be replaced by non-natural amino acids (e.g., non-natural amino acids that comprise olefinic side chains). These non-natural amino acids can form a staple(s) and/or stitch(es) under appropriate conditions, thereby forming stabilized or stapled peptides of these polypeptides. In some cases, the polypeptide is linked to a heterologous moiety (e.g., half-life extending moiety, a linker, and/or a moiety that can allow the polypeptide to traverse the blood brain barrier).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 is a schematic diagram denoting key features of the GDF11 sequence. Signal peptide (residues 1-24), BMP1 type protease and furin-like convertase cleavage sites (arrows), N-terminal prodomain cleavage fragment (residues 25-122), C-terminal prodomain cleavage fragment (residues 123-298), furin recognition sequence (residues 295-298), mature domain (residues 299-407), polyalanine sequence (residues 29-41) and solubility enhancing sequence (residues 60-114, hatched pattern) within the N-terminal prodomain cleavage fragment, single N-linked glycosylation site (N94), single interchain disulfide forming cysteine (C372). Figure discloses SEQ ID NO: 18.

FIG. 2A is a photograph of a SDS-PAGE gel used for characterization of GDF11. Samples (5 μg/lane) were subjected to SDS-PAGE under reducing and non-reducing conditions on 4-20% gradient gels. The gel was stained with Coomassie brilliant blue. Lane 1, full length GDF11; lane 2, full length GDF11 treated with endoproteinase Asp-N for 2.5 hr at 37° C.; lane 3, AspN-treated GDF11 further digested with furin at 37° C. for 6 hr; lane 4, AspN-treated GDF11 digested with furin at 37° C. for 7 hr followed by overnight at 4° C., lane 5, AspN-treated GDF11 digested with furin at 37° C. for 23 hr; lane 6, mature GDF11 standard (0.25 μg) formulated in bovine serum albumin; lane 7, mature GDF11 standard (1 μg) formulated in bovine serum albumin; lane S, molecular weight standards. The molecular masses of molecular weight markers are indicated at the left.

FIG. 2B shows a Coomassie stained gel and a Western blot analysis of full length GDF11 under reducing conditions. The proteins were visualized by western blotting with a polyclonal antibody raised against the C-terminus of mature GDF11. Apparent molecular masses of molecular weight markers are indicated at the left. Arrows denote the stained bands of mature GDF11.

FIG. 2C is a photograph of a SDS-PAGE gel used for characterization of GDF11. Samples (5 μg/lane) were subjected to SDS-PAGE under reducing and non-reducing conditions on 4-20% gradient gels. The gel was stained with Coomassie brilliant blue. Lane 1, full length GDF11 treated with plasmin for 2 hr at room temperature; lane 2, full length GDF11 treated with trypsin for 5 hr at room temperature; lane 3 full length GDF11 treated with furin for 24 hr at 37° C.; lanes 4-7 full length GDF11 treated with endoproteinase Asp-N for 1.5 hr at 37° C.; lane 4 AspN-treated GDF11 no further treatment; lane 5, AspN-treated GDF11 treated with plasmin for 2 hr at room temperature; lane 6, AspN-treated GDF11 treated with trypsin for 5 hr at room temperature; lane 7, AspN-treated GDF11 digested with furin for 24 hr at 37° C.; lane 8, supernatant from lane 7 sample; lane 9, pellet fraction from lane 7 sample, lane 10 mature GDF11 standard (1 μg); lane 11, supernatant from lane 4 sample following 10 min incubation at 95° C. The molecular masses of molecular weight markers are indicated at the left.

FIG. 3A shows the characterization of GDF11 samples by size exclusion chromatography (SEC). Full length GDF11 (100 μg), AspN-treated full length GDF11 (100 μg), and AspN-treated GDF11 digested with furin at 37° C. for 24 hr (500 μg) were subjected to SEC on a Superdex 200 column. The elution profile of gel filtration standards and their molecular masses are also shown. The insert shows column fractions from the AspN/furin digest that were collected and analyzed by SDS-PAGE under non-reducing conditions. Fractions 13-15 that correspond to lanes 3-5 were pooled and subject to analysis by mass spectrometry and activity measurements.

FIG. 3B shows the dissociation rate of the GDF11 prodomain peptide 60-112/114/mature GDF11 complex as assessed by Octet. Samples that were biotinylated through the single N-linked glycan in the prodomain peptide or Asp-N/furin without biotin control, were captured on a Streptavidin sensor for 5 min, washed for 1 min, and dissociation over time was monitored for 30 min. The insert shows binding of the biotinylated GDF11 Asp-N/furin sample at 12.5 μg/mL, 25 μg/mL, 50 μg/mL, 100 μg/mL, and 200 μg/mL.

FIG. 3C shows an Octet analysis of the binding characteristics of the GDF11 prodomain peptide 60-114/mature GDF11 complex, and AspN digested GDF11 for Activin RIB-Fc and Activin RIIA-Fc. Biotinylated GDF11 samples (100 μg/mL) were captured on Streptavidin sensor tips for 5 min, washed for 1 min, then incubated for 15 min with the receptors (20 μg/mL), and dissociation over time was monitored for 5 min. GDF11 Asp-N/furin biotin sample alone, plus Activin RIIA-Fc, or plus Activin RIB-Fc. GDF11 Asp-N biotin sample alone, plus Activin RIIA-Fc, or plus Activin RIB-Fc.

FIG. 4A is a graph showing the time course of GDF11 induced phosphorylation of SMAD 2/3 (to assess the bioactivity of GDF11 on PC12 cells). GDF11 was tested for function in a KIRA assay monitoring SMAD-2/3 phosphorylation.

FIG. 4B is a graph depicting SMAD 2/3 phosphorylation activity of GDF11 test samples shown in FIG. 2A following 60 min treatment. GDF11 was tested for function in a KIRA assay monitoring SMAD-2/3 phosphorylation.

FIG. 4C is a graph showing the signaling activity of GDF11 test samples shown in FIG. 2A in a luciferase reporter assay following 6 hr treatment. GDF11 was tested for function in a SMAD reporter luciferase assay with luciferase expression under the control of the SMAD transcriptional response element (TRE).

FIG. 4D is a graph showing the signaling activity of GDF11 and TGF-β1 with and without treatment of GDF11 prodomain-Fc and TGF-β1 LAP-Fc in a luciferase reporter assay following 6 hr treatment. GDF11 was tested for function in a SMAD reporter luciferase assay in which luciferase expression is under the control of the SMAD transcriptional response element.

FIG. 5A is a Reverse phase chromatography tracing of the reduced and deglycosylated SEC-purified AspN/furin digested GDF11 sample shown in FIG. 3A.

FIG. 5B is a deconvoluted mass spectrum of the 25.0 min-peak from A.

FIG. 5C is a deconvoluted mass spectrum of the 21.3 min-peak from A.

FIG. 5D is a deconvoluted mass spectrum of the 22.1 min-peak from A.

FIG. 5E is a deconvoluted mass spectra of the reduced, deglycosylated, GDF11 treated with endoproteinase Asp-N shown in FIG. 2A, lane 2.

FIG. 6A is a molecular model showing the association of the GDF11 solubility enhancing peptide on the crystal structure of mature GDF11. The crystal structure of latent TGF-β1 (Shi et al., Nature, 474:343-9 (2011)) was superposed on the GDF11 active mature domain (Padyana et al., Acta Crystallogr F Struc Biol Commun., 72:160-164 (2016)) to position the GDF11 α2 helix by using the TGF-β1 α2 helix as an initial position. The GDF11 α1 helix was docked against the GDF11 mature domain dimer using PIPER. See Example 1, Materials and Methods for more details on the model. GDF11 propeptide 66-114, black; mature GDF11, grey.

FIG. 6B is a molecular model of ACE490 propeptide-3xG4S (SEQ ID NO:19; black)-mature GDF11 (grey) in side view.

FIG. 6C is a molecular model of ACE490 propeptide-3xG4S (SEQ ID NO:19; black)-mature GDF11 (grey) in top view.

FIG. 6D is a schematic diagram showing AspN/furin cleavage-induced precipitation of prodomain peptide 122-299.

FIG. 7A shows a Coomassie blue stained SDS-PAGE analysis of conditioned medium for the fusion proteins indicated. Apparent molecular masses of molecular weight markers are indicated at the right. Arrow denotes the position of stained bands of prodomain/mature GDF11 fusions. Samples were analyzed under non-reducing conditions.

FIG. 7B is a Western blot of fusions visualized using an anti-GDF11 C-terminal peptide polyclonal antibody for detection. Apparent molecular masses of molecular weight markers are indicated at the right. Samples were analyzed under reducing conditions.

FIG. 8A is a schematic diagram summarizing the redox steps used to induce dimerization of propeptide/mature GDF11 domain fusion proteins.

FIG. 8B is a Coomassie blue stained SDS-PAGE analysis of purified ACE490 proteins before and after redox under reducing and non-reducing conditions. Apparent molecular masses of molecular weight markers are indicated at the right. Lane 1, ACE490 alone; Lane 2, ACE490 plus DTT and Aldrithiol™; Lane 3, ACE490 plus DTT and Aldrithiol™ in 1 M guanidine HCl; Lane 4, molecular weight markers.

FIG. 8C is a graph showing signaling activity of ACE490 GDF11 test samples in luciferase reporter assay following 6 hr treatment.

FIG. 8D is a Coomassie blue stained SDS-PAGE analysis of purified ACE498 proteins before and after redox and after treatment with AspN under reducing and non-reducing conditions. Apparent molecular masses of molecular weight markers are indicated at the right. Lane 1, ACE498 alone; Lane 2, ACE498 plus DTT and Aldrithiol™ for 20 hr at room temperature; Lane 3, ACE490 plus DTT/Aldrithiol™ and then treated with AspN; Lane 4, MW markers.

FIG. 8E is a graph depicting the signaling activity of ACE498 GDF11 test samples in luciferase reporter assay following 6 hr treatment.

FIG. 9A is a SDS-PAGE analysis of mature huGDF11 in the presence and absence of a synthetic propeptide. Lane 1, huGDF11-mature, 0.5 ug; Lane 2, pH 8.5; Lane 3, +Peptide, pH 8.5; Lane 4, pH 7.5; Lane 5, +Peptide, pH 7.5; Lane 6, MW Marker; Lane 7, huGDF11-mature+DTT; Lane 8, pH 8.5+DTT; Lane 9, +Peptide, pH 8.5+DTT; Lane 10, pH 7.5; Lane 11, +Peptide, pH 7.5+DTT; 12, MW Marker.

FIG. 9B is a SDS-PAGE analysis of mature huGDF11 in the presence and absence of a synthetic propeptide. Lane 1, huGDF11-mature 2 μg; Lane 2, pH 6.5; Lane 3, +Peptide, pH 6.5; Lane 4, pH 7.5; Lane 5, +Peptide, pH 7.5; Lane 6, MW Marker.

FIG. 10A provides two secondary structure predictions for the indicated prodomain sequences of human GDF11 using PSIPRED (1999, Jones et al., Journal of Molecular Biology, 292, 195-202 (1999) and the more recent PSIPRED (2013, Buchan et al., Nucleic Acids Research, 41 (W1): W340-W348 (2013). [symbol] =identities in α1 helices, [symbol] =cysteine that was mutated into serine in the crystallization product for the porcine TGF-β1 (Shi et al., Nature, 474:343-349 (2011)). The vertical bar “|” within the sequence indicates the first AspN cleavage site between L114 and D115. Shi_et_al: assignment of secondary structure elements and curated alignment of porcine TGF-β1 and human GDF11 according to Shi et al., Nature, 474:343-349 (2011). Figure discloses SEQ ID NOS 61-62, respectively, in order of appearance.

FIG. 10B provides secondary structure predictions for the indicated prodomain sequences of porcine TGF-β1: Sequences were analyzed as described in FIG. 10A. [symbol] =identities in α1 helices, [symbol] =cysteine that was mutated into serine in the crystallization product for the porcine TGF-β1 (Shi et al., Nature, 474:343-349 (2011)). Shi-_et_al: assignment of secondary structure elements of porcine TGF-β1 according to Shi et al., Nature, 474:343-349 (2011). Figure discloses SEQ ID NO: 63.

FIG. 11 shows synthetic peptides designed to form a complex with mature GDF11; (*) put=putative lasso helix, the putative helices (grey-shaded) follow the secondary structure predictions of PSIPRED (2013, Buchan et al., Nucleic Acids Research, 41 (W1):W340-W348 (2013)) in FIG. 10A (REVVKQL (SEQ ID NO:33)), to which I and S were N-terminally prepended based on their intermediate helix propensity values (Pace et al., Biophysical J, 75, 422-427 (1998)), (ISREVVKQL (SEQ ID NO:25)). Figure discloses SEQ ID NOS 64, 21 and 28-32, respectively, in order of appearance.

FIG. 12 provides secondary structure predictions for the first 132 residues of the human GFD11 pro-domain, according to PSIPRED (1999, Jones et al., Journal of Molecular Biology, 292, 195-202 (1999)) and PSIPRED (2013, Buchan et al., Nucleic Acids Research, 41 (W1): W340-W348 (2013)) as in FIGS. 10A and 10B. Figure discloses SEQ ID NO: 65.

FIG. 13 provides an alignment following curated alignment comparing α1 and α2 helices and latency lassos of porcine TGF-β1, human GDF11, and human BMP7; grey shaded=residues identical in the al helices of porcine TGF-β1 and human GDF11 prodomains, respectively. Figure discloses SEQ ID NOS 66-68, respectively, in order of appearance.

FIG. 14 provides the results of an NCBI Protein Blast search for Protein Data Bank entries homologous to a putative helix sequence of human GDF11 prodomain peptide 92-107 (sequence APNISREVVKQLLPKA (SEQ ID NO:23)). Figure discloses SEQ ID NOS 23, 25, 69-73, 70, 74-76, 33 and 77-89, respectively, in order of appearance.

FIG. 15 provides an alignment of GDF11 amino sequences from mouse, human, rat, zebra fish, chimpanzee, and cow. These alignments are helpful in determining which amino acids in a GDF11 sequence can be mutated without affecting the structure and/or activity of GDF11. For example, amino acids at positions that are different in one or more of the GDF11 sequences can be substituted. The substitutions can be conservative substitutions.

### DETAILED DESCRIPTION

This disclosure is based, in part, on Applicant's several surprising findings regarding GDF11. First, proteolytic activation of full length GDF11 in vitro involves two steps: (i) initial cleavage at Asp-122; and (ii) step (i) followed by cleavage at a cleavage site between the GDF11 prodomain and the GDF11 mature domain. Second, certain prodomain fragments of GDF11 can improve the solubility of the mature domain of GDF11 at neutral pH without inactivating the GDF11 mature domain. Accordingly, this disclosure features solubility enhancing polypeptides of GDF11 that permit GDF11 polypeptides to be soluble at neutral pH and still remain active. Thus, even if the GDF11 polypeptides are stored in an acid formulation, they can be administered to a human subject and the neutral pH blood would not render the GDF11 polypeptides insoluble. The disclosure also provides methods of making such polypeptides and methods of using same.

### GDF11

The full length sequence of human GDF11 is shown below.

(SEQ ID NO: 1)
mvlaaplllgflllalelrprgeaAEGPAAAAAAAAAAAAAGVGGERSSR
PAPSVAPEPDGCPVCVWRQHSRELRLESIKSQILSKLRLKEAPNISREVV
KQLLPKAPPLQQILDLHDFQG [symbol] ALQPEDFLEEDEYHATTETVISMAQET
DPAVQTDGSPLCCHFHFSPKVMFTKVLKAQLWVYLRPVPRPATVYLQILR
LKPLTGEGTAGGGGGGRRHIRIRSLKIELHSRSGHWQSIDFKQVLHSWFR
QPQSNWGIEINAFDPSGTDLAVTSLGPGAEGLHPFMELRVLENTKRSRRN
LGLDCDEESSESROCRYPLTVDFEAFGWDWIIAPKRYKANYCSGQCEYME
MQKYPETHLVQQANPRGSAGPCCIPTKMSPINMLYENDKQQIIYGKIPGM
VVDRCGCS

The signal peptide (amino acids 1-24) is shown in lower case; the propeptide domain is amino acids 25-298; the mature domain (amino acids 299-407) is boldened/underlined, and an exemplary solubility enhancing sequence (amino acids 60-114) is underlined.

The nucleic acid sequence encoding human GDF11 (entire open reading frame including the signal sequence) is provided below:

(SEQ ID NO: 2)
ATGGTTCTTGCAGCTCCTCTGCTGCTGGGCTTCCTGCTCCTCGCTCTGGA
GCTGCGACCCAGAGGTGAGGCGGCCGAGGGACCTGCAGCTGCAGCTGCTG
CTGCAGCTGCCGCAGCAGCTGCAGGGGTCGGAGGAGAGCGCTCCAGCCGG
CCAGCTCCTTCTGTGGCTCCTGAGCCAGACGGCTGCCCCGTGTGCGTTTG
GCGGCAGCACAGCCGCGAGCTGCGCCTAGAGAGCATCAAGTCGCAGATCC
TGAGCAAACTGCGGCTCAAGGAGGCGCCCAACATCAGCCGCGAGGTGGTG
AAGCAGTTGCTGCCCAAGGCGCCGCCGCTGCAACAGATCCTGGACCTACA
CGACTTCCAGGGCGACGCGCTGCAACCCGAGGACTTCCTGGAGGAGGACG
AGTACCACGCCACCACCGAGACCGTCATTAGCATGGCCCAGGAGACGGAC
CCAGCAGTACAGACAGATGGCAGCCCTCTCTGCTGCCATTTCCACTTCTC
ACCCAAGGTGATGTTCACAAAGGTCCTGAAGGCCCAGTTGTGGGTGTACC
TACGGCCTGTACCCCGCCCAGCCACAGTCTACCTGCAAATCCTGCGACTA
AAACCCCTAACTGGGGAAGGGACCGCAGGTGGAGGTGGTGGAGGCCGGCG
TCACATCCGTATCCGCTCACTGAAGATTGAGCTGCACTCACGCTCAGGCC
ATTGGCAGAGCATCGACTTCAAGCAAGTGCTACACTCTTGGTTCCGCCAG
CCACAGAGCAACTGGGGCATCGAGATCAACGCCTTTGATCCCAGTGGTAC
AGACCTGGCTGTTACATCTCTGGGGCCGGGAGCCGAGGGGCTGCATCCAT
TCATGGAGCTTCGAGTCCTAGAGAACACAAAACGTTCCCGGCGGAACCTG
GGTCTGGACTGCGACGAGCACTCAAGCGAGTCCCGCTGCTGTAGGTATCC
TCTCACAGTGGACTTTGAGGCTTTCGGCTGGGACTGGATCATCGCACCTA
AGCGCTACAAGGCCAACTACTGCTCCGGCCAGTGCGAGTACATGTTCATG
CAGAAGTACCCGCATACCCATTTGGTGCAGCAGGCCAATCCAAGAGGCTC
TGCTGGACCCTGTTGTACCCCTACCAAGATGTCCCCAATCAACATGCTCT
ACTTCAACGACAAGCAGCAGATCATCTACGGCAAGATCCCTGGCATGGTG
GTGGATCGCTGTGGCTGCTCTTGA

This sequence is part of the ACE378 construct described in the Examples.

The amino acid and nucleic acid sequences of GDF11 proteins from other species, e.g., cow, dog, cat, chicken, mouse, rat, pig, turkey, horse, fish, baboon, gorilla, are well known in the art (see, NCBI, Protein database).

### Methods for Activating GDF11 Proteins

The disclosure features a method for producing an activated GDF11 protein. The method involves contacting a GDF11 protein with a first protease that cleaves at a BMP1 site of the GDF11 protein and a second protease that cleaves between the prodomain and the mature domain of GDF11. In some instances, the first protease and the second protease are added at the same or substantially the same time to the GDF11 protein that needs to be activated. In some instances, the first protease is added before adding the second protease to the GDF11 protein. In certain instances, the GDF11 is human GDF11. In a specific embodiment, the human GDF11 has an amino acid sequence that is at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence set forth in SEQ ID NO:1. In some embodiments, the human GDF11 protein has one to ten (e.g., 1, 2, 3, 4, 5, 6, 7, 8, 9, or 10) amino acid substitutions, insertions, and/or deletions within SEQ ID NO:1. In certain instances the GDF11 protein is linked to a heterologous moiety (e.g., a half-life extending moiety, a moiety that helps the protein traverse the blood brain barrier). In some instances, the first protease is an endoproteinase. In a specific embodiment, the protease that cleaves at the BMP1 site of the GDF11 protein is endoproteinase AspN. In some instances, the second protease is furin. In other instances, the second protease is plasmin. In yet other embodiments, the second protease is trypsin.

When full length GDF11 proteins were recombinantly expressed in cells, the recombinant GDF11 protein was found to be inactive. In order to activate GDF11, the enzyme furin was added; however, furin was unable to promote cleavage at the cleavage site between the prodomain and the mature domain. When the GDF11 was first treated with a protease that can cleave at the aspartic acid residue at position of 122 of SEQ ID NO:1, it was surprisingly found that GDF11 then became susceptible to cleavage at the cleavage site between the prodomain and the mature domain of GDF11. Thus, also provided are methods of producing an activated GDF11 protein, wherein the GDF11 is expressed in a host cell. The GDF11 from the host cell is isolated and contacted with a first protease that cleaves at a BMP1 site of the GDF11 protein and a second protease that cleaves between the prodomain and the mature domain of GDF11. In some instances, the first protease and the second protease are added at the same or substantially the same time to the GDF11 protein that needs to be activated. In some instances, the first protease is added before adding the second protease to the GDF11 protein. In certain instances, the GDF11 is human GDF11. In certain instances, the GDF11 is human GDF11. In a specific embodiment, the human GDF11 has an amino acid sequence that is at least 75%, at least 80%, at least 85%, at least 90%, at least 95%, at least 97%, or 100% identical to the amino acid sequence set forth in SEQ ID NO:1. In some embodiments, the human GDF11 protein has one to ten amino acid substitutions, insertions, and/or deletions within SEQ ID NO:1. In some instances, the first protease is an endoproteinase. In a specific embodiment, the protease that cleaves at the BMP1 site of the GDF11 protein is endoproteinase AspN. In some instances, the second protease is furin. In other instances, the second protease is plasmin. In yet other embodiments, the second protease is trypsin. In certain instances, the host cell is a microbial cell (e.g., E. coli (see, e.g., Kamionka M, Curr Pharm Biotechnol., 12(2):268-274 (2001)). In certain instances, the host cell is a yeast cell (e.g., Pichia pastoris; Saccharomyces cerevisiae). In other embodiments, the host cell is an insect cell or a baculovirus-infected insect cell (see, e.g., Hu Y., Acta Pharmacol Sin. 26:405-16 (2005); Jarvis D., Methods Enzymol., 463:191-222 (2009)). In yet other embodiments, the host cell is a mammalian cell (e.g., CHO, COS, 293, or NIH3T3 cells). In certain instances, the host cell is a fungal cell (e.g., Aspergillus). In other instances, the host cell is an algal cell (see, e.g., Handbook of Microalgal Culture: Applied Phycology and Biotechnology, Second Edition (2013), published by Blackwell Publishing Ltd.).

The activated GDF11 protein can, if desired, be formulated as a sterile pharmaceutical composition. The pharmaceutical composition can have a pH in the range of about 5.0 to about 6.5. In a specific embodiment, the pharmaceutical composition has a pH of about 5.5. The term “about” as used herein means the recited pH value ±0.2. Thus a pH of “about 5.5” means a pH of 5.3, 5.4, 5.5, 5.6, and 5.7.

### Multimeric GDF11 Proteins

Provided herein are multimeric GDF11 proteins that are soluble and active at neutral pH. The multimeric protein comprises two polypeptides comprising the mature domain of GDF11 (e.g., 296-407 or 299-407 of SEQ ID NO:1) and at least one (one or two) polypeptides comprising prodomain fragments of GDF11. In some cases, the prodomain fragments of GDF11 of the multimeric protein have the same amino acid sequence. In other embodiments, the prodomain fragments of GDF11 of the multimeric protein are different (e.g., 60-112 and 60-114; 60-112 and 60-117; or 60-114 and 60-117 of SEQ ID NO:1). In some instances, the multimeric GDF11 protein comprises polypeptides from human GDF11. In some embodiments, the mature domain comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the mature domain of human GDF11—i.e., amino acids 296-407 or 299-407 of human GDF11 (SEQ ID NO:1). In a particular embodiment, the mature domain comprises an amino acid sequence that is identical to the mature domain of human GDF11 (296-407 or 299-407 of SEQ ID NO:1). In some cases, the polypeptide or polypeptides comprising prodomain fragments of GDF11 comprise or consist of an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to amino acids 60-112 of SEQ ID NO:1. In some embodiments, the asparagine at the position corresponding to position 94 of SEQ ID NO:1 is glycosylated in one or both polypeptides comprising prodomain fragments of GDF11. In some cases, the polypeptide or polypeptides comprising prodomain fragments of GDF11 comprise amino acids 60-112 of SEQ ID NO:1 with at least one to ten (e.g., 1, 2, 3, 4, 5, 6, 7, 8, 9, or 10) substitutions, deletions, or insertions. In certain instances, the polypeptide or polypeptides comprising prodomain fragments of GDF11 consist of amino acids 60-112 of SEQ ID NO:1. In some instances, the polypeptide or polypeptides comprising prodomain fragments of GDF11 comprise amino acids 60-114 of SEQ ID NO:1 with at least one to ten (e.g., 1, 2, 3, 4, 5, 6, 7, 8, 9, or 10) substitutions, deletions, or insertions. In other instances, the polypeptide or polypeptides comprising prodomain fragments of GDF11 consist of amino acids 60-114 of SEQ ID NO:1. In some instances, the polypeptide or polypeptides comprising prodomain fragments of GDF11 comprise amino acids 60-117 of SEQ ID NO:1 with at least one to ten (e.g., 1, 2, 3, 4, 5, 6, 7, 8, 9, or 10) substitutions, deletions, or insertions. In yet other instances, the polypeptide or polypeptides comprising prodomain fragments of GDF11 consist of amino acids 60-117 of SEQ ID NO:1. In certain cases, the multimeric protein comprises prodomain fragments of GDF11 consisting of amino acids 60-112 and 60-117 of SEQ ID NO:1. In certain cases, the multimeric protein comprises prodomain fragments of GDF11 consisting of amino acids 6-112 and 60-114 of SEQ ID NO:1. In certain cases, the multimeric protein comprises prodomain fragments of GDF11 consisting of amino acids 6-114 and 60-117 of SEQ ID NO:1.

Also provided are multimeric GDF11 proteins that comprise a first polypeptide and a second polypeptide. In some cases, the multimeric protein comprises a dimer of the first polypeptide and at least one of the second polypeptide. In some cases, the multimeric protein comprises a dimer of the first polypeptide and two of the second polypeptide. The second polypeptide non-covalently associates with each monomer of the dimer of the first polypeptide. In certain embodiments, the first and second polypeptide are human GDF11 polypeptides. The first polypeptide comprises or consists of an amino acid sequence of the mature domain of GDF11. In certain embodiments, the first polypeptide comprises or consists of an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or identical to amino acids 296-407 or 299-407 of human GDF11 (SEQ ID NO:1). In certain embodiments, the first polypeptide has one to ten (one, two, three, four, five, six, seven, eight, nine, or ten) substitutions, deletions or insertions in amino acids 296-407 or 299-407 of human GDF11 (SEQ ID NO:1). In some embodiments, the cysteine residues in the mature domain are not altered. In certain instances, one to three amino acids are deleted at the N and/or C-terminus of amino acids 296-407 or 299-407 of human GDF11 (SEQ ID NO:1). In certain instances, one to five amino acids are substituted within amino acids 296-407 or 299-407 of human GDF11 (SEQ ID NO:1). The substitutions may be conservative or non-conservative. The first polypeptide can be 109, 108, 107, 106, 105, 104, 103, 102, 101, 100, 99, 98, 97, 96, 95, 94, 93, 92, 91, or 90 amino acids in length. The second polypeptide comprises RELRLESIKSQILSKLRLKG (SEQ ID NO:51) or a stabilized peptide thereof. The second polypeptide can be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In certain embodiments, the second polypeptide comprises or consists of SPRELRLESIKSQILSKLRLKG (SEQ ID NO:32) or a stabilized peptide thereof. In certain embodiments, the second polypeptide comprises or consists of SPRELRLESIKSQILSKLRLKEAPNIS (SEQ ID NO:29) or a stabilized peptide thereof. In certain embodiments, the second polypeptide comprises or consists of DGCPVCVWRQHSRELRLESIKSQILSKLRLKG (SEQ ID NO:31) or a stabilized peptide thereof. In certain embodiments, the second polypeptide comprises or consists of DGCPVCVWRQHSRELRLESIKSQILSKLRLKEAPNIS (SEQ ID NO:30) or a stabilized peptide thereof. In certain embodiments, the second polypeptide comprises or consists of SPRELRLESIKSQILSKLRLKEAPNISREVVKQLLPKAPP (SEQ ID NO:28) or a stabilized peptide thereof. In other embodiments, the second polypeptide comprises or consists of SPRELRLESIKSQILSKLRLKEAPNISREVVKQLLPKAPPLQQIL (SEQ ID NO:21) or a stabilized peptide thereof. The stabilized peptide can be made by replacing at least two (e.g., 2, 3, 4, 5, 6, 7, or 8) amino acids of the above-listed sequences that are separated by 2, 3, or 6 amino acids with non-natural amino acids with olefin-containing side chains that can be covalently joined, e.g., using a ring-closing metathesis reaction. In some embodiments, two amino acids (separated by 2, 3, or 6 amino acids) of each of the above sequences is replaced with non-natural amino acids with olefin-containing side chains.

One or more of the polypeptides of the multimeric protein described above can be linked to a half-life extending moiety. Such half-life extending moieties are discussed further below.

One or more of the polypeptides of the multimeric protein described above can be linked to a moiety that can assist the multimeric protein traverse the blood brain barrier. Such moieties are discussed further below.

The activity of the multimeric GDF11 protein can be assayed according to any method known in the art. In one instance, the activity of the multimeric GDF11 protein is assayed using the Kinase Induced Receptor Activation Assay. In another embodiment, the activity of the multimeric GDF11 protein is assayed using reporter assays in SMAD2/3 reporter cells. In yet another embodiment, the activity of the multimeric GDF11 protein is assayed using a SMAD2/3 phosphorylation assay.

### GDF11 Fusion Proteins

Also featured herein are fusion proteins linking a prodomain fragment of GDF11 with the mature domain of GDF11. The two sequences of GDF11 can be linked or conjugated together by any method known in the art, including the use of peptide linkers. For example, the fusion protein can comprise, in order, a first GDF11 amino acid sequence and a second GDF11 amino acid sequence linked directly via a peptide linker of 5 to 150 amino acids in length. In some instances, the peptide linker is 10 to 20, 10 to 30, 10 to 40, 10 to 50, 10 to 100, 10 to 144, or 10 to 150 amino acids in length. In certain embodiments, the linker is 15 amino acids in length. In other embodiments, the linker is 20 amino acids in length. In yet other embodiments, the linker is 144 amino acids in length. Linkers are discussed further below.

In some embodiments, the first amino acid sequence is 52 to 65 amino acids in length and comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acids 60 to 114 of SEQ ID NO:1. In some embodiments, the first amino acid sequence is 52 to 65 amino acids in length and comprises amino acids 60 to 114 of SEQ ID NO:1 with one to ten amino acid substitutions, deletions, and/or insertions. In certain instances, one to six amino acids of the first amino acid sequence are substituted with non-natural amino acids. Such non-natural amino acids can be inserted at positions 3 and/or 6 amino acids apart and can facilitate the formation of “stapled” peptides. In certain instances, one or more of methionine residues in the first amino acid sequence are replaced with norleucine. In some embodiments, the first amino acid sequence is 52 to 65 amino acids in length and comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acids 71 to 123 of SEQ ID NO:1. In some embodiments, the first amino acid sequence is 52 to 65 amino acids in length and comprises amino acids 71 to 123 of SEQ ID NO:1 with one to ten amino acid substitutions, deletions, and/or insertions. In some embodiments, the first amino acid sequence is 52 to 62 amino acids in length. In some embodiments, the first amino acid sequence is 52 to 55 amino acids in length. In some embodiments, the first amino acid sequence is 52, 53, 54, 55, 56, 57, 58, 59, 60, 61, 62, 63, 64, or 65 amino acids in length. In one embodiment, the first amino acid sequence consists of amino acids 71-123 of SEQ ID NO:1. In another embodiment, the first amino acid sequence consists of amino acids 60-122 of SEQ ID NO:1. In yet another embodiment, the first amino acid sequence consists of amino acids 60-114 of SEQ ID NO:1. In certain embodiments, the first amino acid sequence is a stabilized polypeptide (e.g., a stapled polypeptide based on amino acids 60-114 of SEQ ID NO:1, or amino acids 71-123 of SEQ ID NO:1, or amino acids 60-122 of SEQ ID NO:1). In certain embodiments, the first amino acid sequence comprises an alpha-helical region from a non-GDF11 protein. In such instances, the first amino acid sequence may be 52 to 65 amino acids in length.

In some embodiments, the second amino acid sequence comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acids 296-407 or 299-407 of SEQ ID NO:1. In some embodiments, the second amino acid sequence comprises or consists of amino acids 296-407 or 299-407 of SEQ ID NO:1 with one to ten amino acid substitutions, insertions, or deletions.

In one embodiment, the fusion protein comprises or consists of an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acids 35-211 of SEQ ID NO:6. In another embodiment, the fusion protein comprises or consists of an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acids 36-217 of SEQ ID NO:14. In another embodiment, the fusion protein comprises or consists of an amino acid sequence that is at least 70%, at least 75%, at least 80%, at least 85%, at least 90%, at least 95%, at least 97%, or 100% identical to amino acids 36-227 of SEQ ID NO:5. In yet another embodiment, the fusion protein comprises or consists of an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to 36-219 of SEQ ID NO:9.

The fusion proteins described herein may require activation by dimerization, or by both dimerization and proteolytic cleavage. Thus, in certain instances the fusion protein is subjected to a disulfide reducing agent to create a first composition. The first composition is then divided into a second and a third composition. The second composition is contacted with a cysteine activating agent to create a fourth composition. The fourth composition is then combined with the third composition to create a fifth composition. The fifth composition can be tested for activity as described above. If the fifth composition is not active or has low activity, the fifth composition can be treated with a protease that cleaves at the BMP1 site of the protein, thereby making an activated protein. In certain embodiments, the fifth composition is active. In certain embodiments, the disulfide reducing agent is dithiothreitol (DTT). In certain embodiments, the cysteine activating agent is aldrithiol. In some embodiments, the protease that cleaves at the BMP1 site of the protein is endoproteinase AspN. The composition comprising the activated protein (the fourth or fifth composition) may be formulated as a sterile pharmaceutical composition. In certain instances, the formulation has a pH of about 5.0 to about 5.5. In certain instances, the formulation has a pH of about 5.5.

In certain instances, the fusion proteins described herein are produced recombinantly in host cells. In certain instances, the host cell is a microbial cell (e.g., E. coli). In certain instances, the host cell is a yeast cell (e.g., Pichia pastoris; Saccharomyces cerevisiae). In other embodiments, the host cell is an insect cell or a baculovirus-infected insect cell. In yet other embodiments, the host cell is a mammalian cell (e.g., CHO, COS, 293, NIH 3T3 cells). In other embodiments, the host cell is a filamentous fungal cell (e.g., Aspergillus niger, Aspergillus nidulans, Aspergillus oryzae, Trichoderma reesei). In other embodiments, the host cell is an algal cell, such as a microalgal cell (e.g., Chlamydomonas reinhardtii, Scenedesmus obliquus).

The fusion proteins described above can be linked to a half-life extending moiety. Such half-life extending moieties are discussed further below. The fusion proteins described above can also be linked to a moiety that can assist the protein traverse the blood brain barrier. Such moieties are discussed further below.

### Pharmaceutical Compositions

The multimeric protein or chimeric proteins described herein can be formulated as a pharmaceutical composition. In certain instances, the pharmaceutical composition is a sterile formulation that has a pH of about 5.0 to about 5.5. In certain instances, the pharmaceutical composition is a sterile formulation that has a pH of about 5.5. When such pharmaceutical compositions are administered to a human subject in need thereof, even if the formulation is stored at an acidic pH, when the pH is raised to neutral pH (e.g., upon entry into the human body), surprisingly, the multimeric protein or chimeric proteins described above remain soluble and active.

In certain instances, the pharmaceutical composition comprises a pharmaceutically acceptable carrier and a population of dimeric GDF11 proteins. The dimeric GDF11 proteins in the population comprise two GDF11 monomers each of which consists of an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acids 296-407 or 299-407 of human GDF11 (SEQ ID NO:1). In certain cases, the GDF11 monomers have one to ten substitutions, insertions, and/or deletions in amino acids 296-407 or 299-407 of human GDF11 (SEQ ID NO:1). In some cases, none of the cysteine residues of each monomer is substituted or deleted. In some case one to three amino acids are deleted at the N- and/or C-terminus of amino acids 299-407 of human GDF11 (SEQ ID NO:1). At least 70%, at least 75%, at least 80%, at least 85%, at least 90%, at least 95%, or at least 97% of the dimeric GDF11 proteins in the population comprise a polypeptide non-covalently associated with each GDF11 monomer. The polypeptide comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acids 60-112 of SEQ ID NO:1. The population of GDF11 proteins of the pharmaceutical composition are active. For example, they can induce SMAD 2/3 phosphorylation in a Kinase Induced Receptor Activation Assay. In one case, the polypeptide consists of amino acids 60-112 of SEQ ID NO:1 and each GDF11 monomer consists of amino acids 296-407 or 299-407 of SEQ ID NO:1. In another case, the polypeptide consists of amino acids 60-114 of SEQ ID NO:1 and each GDF11 monomer consists of amino acids 296-407 or 299-407 of SEQ ID NO:1. In yet another case, the polypeptide consists of amino acids 60-117 of SEQ ID NO:1 and each GDF11 monomer consists of amino acids 296-407 or 299-407 of SEQ ID NO:1. In certain embodiments, the asparagine at position 94 of SEQ ID NO:1 is glycosylated in the polypeptide.

In other instances, the pharmaceutical composition comprises a pharmaceutically acceptable carrier and a population of dimeric GDF11 proteins. The dimeric GDF11 proteins in the population comprise two GDF11 monomers each of which consists of an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acids 296-407 or 299-407 of human GDF11 (SEQ ID NO:1). In certain cases, the GDF11 monomers have one to ten substitutions, insertions, and/or deletions in amino acids 296-407 or 299-407 of human GDF11 (SEQ ID NO:1). In some cases, none of the cysteine residues of each monomer is substituted or deleted. In some case one to three amino acids are deleted at the N- and/or C-terminus of amino acids 296-407 or 299-407 of human GDF11 (SEQ ID NO:1). At least 70%, at least 75%, at least 80%, at least 85%, at least 90%, at least 95%, or at least 97% of the dimeric GDF11 proteins in the population comprise a polypeptide non-covalently associated with each GDF11 monomer. The polypeptide comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to any one of SEQ ID NO:21, SEQ ID NO:28, SEQ ID NO:29, SEQ ID NO:30, SEQ ID NO:31, or SEQ ID NO:32. The population of GDF11 proteins of the pharmaceutical composition are active. For example, they can induce SMAD 2/3 phosphorylation in a Kinase Induced Receptor Activation Assay. In certain embodiments, the asparagine at position 94 of SEQ ID NO:1 is glycosylated in the polypeptide.

Method of Preparing a GDF11 Protein Formulation with a Synthetic Propeptide

The disclosure also provides a method of preparing a GDF11 protein formulation comprising a first polypeptide and a second polypeptide to improve solubility.

The first polypeptide can be a polypeptide that comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to any one of SEQ ID NO:21, SEQ ID NO:28, SEQ ID NO:29, SEQ ID NO:30, SEQ ID NO:31, or SEQ ID NO:32. In certain embodiments, the first polypeptide is 22 to 50 amino acids in length. In some embodiments, the first polypeptide is a stapled peptide (e.g., a hydrocarbon-stapled peptide). In certain cases, the first polypeptide is a non-GDF11 polypeptide that comprises an alpha-helical sequence.

The second polypeptide comprises or consists of an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acids 296-407 or 299-407 of human GDF11 (SEQ ID NO:1). In certain cases, the second polypeptide has one to ten substitutions, insertions, and/or deletions in amino acids 296-407 or 299-407 of human GDF11 (SEQ ID NO:1). In some cases, none of the cysteine residues of the second polypeptide is substituted or deleted. In some case one to three amino acids are deleted at the N- and/or C-terminus of amino acids 296-407 or 299-407 of human GDF11 (SEQ ID NO:1).

The polypeptides described above can be formulated as a pharmaceutical composition. In certain instances, the pharmaceutical composition is a sterile formulation that has a pH of about 5.0 to about 5.5. In certain instances, the pharmaceutical composition is a sterile formulation that has a pH of about 5.5. Such pharmaceutical compositions remain soluble and active when the pH of the formulation is raised to neutral pH (e.g., upon administration to a human subject).

### Linkers

There is no particular limitation on the linkers that can be used in the polypeptide constructs described above. In some embodiments, the linker is a peptide linker. In some embodiments, any arbitrary single-chain peptide comprising about one to 30 residues (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids) can be used as a linker. In other embodiments, the linker is 10 to 20, 10 to 30, 10 to 40, 10 to 50, 10 to 60, 10 to 70, 10 to 80, 10 to 90, 10 to 100, 10 to 144, or 10 to 150 amino acids in length. In certain instances, the linker contains only glycine and/or serine residues. Examples of such peptide linkers include: Gly, Ser; Gly Ser; Gly Gly Ser; Ser Gly Gly; Gly Gly Gly Ser (SEQ ID NO:34); Ser Gly Gly Gly (SEQ ID NO:35); Gly Gly Gly Gly Ser (SEQ ID NO:4); Ser Gly Gly Gly Gly (SEQ ID NO:36); Gly Gly Gly Gly Gly Ser (SEQ ID NO:37); Ser Gly Gly Gly Gly Gly (SEQ ID NO:38); Gly Gly Gly Gly Gly Gly Ser (SEQ ID NO:39); Ser Gly Gly Gly Gly Gly Gly (SEQ ID NO:40); (Gly Gly Gly Gly Ser)ₙ (SEQ ID NO:4)n, wherein n is an integer of one or more; and (Ser Gly Gly Gly Gly)ₙ (SEQ ID NO:36)n, wherein n is an integer of one or more. In some instances, the linker has the amino acid sequence of SEQ ID NO:4 with the exception that the serine residue is replaced with another amino acid. In some instances, the linker has multiple copies (e.g., 1, 2, 3, 4, 5, 6, 7, 8, 9, 10) of the amino acid sequence of SEQ ID NO:4 with the exception that the serine residue in each copy of the linker is replaced with another amino acid.

In other embodiments, the linker peptides are modified such that the amino acid sequence GSG (that occurs at the junction of traditional Gly/Ser linker peptide repeats) is not present. For example, the peptide linker comprise an amino acid sequence selected from the group consisting of: (GGGXX)ₙGGGGS (SEQ ID NO:41) and GGGGS(XGGGS)ₙ (SEQ ID NO:42), where X is any amino acid that can be inserted into the sequence and not result in a polypeptide comprising the sequence GSG, and n is 0 to 4. In one embodiment, the sequence of a linker peptide is (GGGX₁X₂)ₙGGGGS and X₁ is P and X₂ is S and n is 0 to 4 (SEQ ID NO:43). In another embodiment, the sequence of a linker peptide is (GGGX₁X₂)ₙGGGGS and X₁ is G and X₂ is Q and n is 0 to 4 (SEQ ID NO:44). In another embodiment, the sequence of a linker peptide is (GGGX₁X₂)ₙGGGGS and X₁ is G and X₂ is A and n is 0 to 4 (SEQ ID NO:45). In yet another embodiment, the sequence of a linker peptide is GGGGS(XGGGS)ₙ, and X is P and n is 0 to 4 (SEQ ID NO:46). In one embodiment, a linker peptide of the invention comprises or consists of the amino acid sequence (GGGGA)₂GGGGS (SEQ ID NO:47). In another embodiment, a linker peptide comprises or consists of the amino acid sequence (GGGGQ)₂GGGGS (SEQ ID NO:48). In yet another embodiment, a linker peptide comprises or consists of the amino acid sequence (GGGPS)₂GGGGS (SEQ ID NO:49). In a further embodiment, a linker peptide comprises or consists of the amino acid sequence GGGGS(PGGGS)₂ (SEQ ID NO:50).

In another embodiment, the linker is an XTEN. For example, the linker can be AE 144.

In certain embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), di sulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

### Half-Life Extending Moieties

In some embodiments, one or more polypeptides described herein can comprises at least one heterologous moiety that is a “half-life extending moiety.” Half-life extending moieties, can comprise, for example, (i) XTEN polypeptides; (ii) Fc; (iii) human serum albumin (HSA), (iv) albumin binding polypeptide or fatty acid, (v) the C-terminal peptide (CTP) of the β subunit of human chorionic gonadotropin, (vi) proline-alanine-serine polymer (PAS); (vii) homo-amino acid polymer (HAP); (viii) human transferrin; (ix) polyethylene glycol (PEG); (x) hydroxyethyl starch (HES), (xi) polysialic acids (PSAs); (xii) a clearance receptor or fragment thereof which blocks binding of the chimeric molecule to a clearance receptor; (xiii) low complexity peptides; (xiv) vWF; (xv) elastin-like peptide (ELP) repeat sequence; (xvi) fusion with artificial GLK; or (xv) any combinations thereof. See, Strohl, BioDrugs, 29:215-239 (2015), incorporated by reference herein in its entirety.

In some embodiments, the half-life extending moiety comprises or consists of an XTEN polypeptide. Non-limiting, examples of XTENs are disclosed in U.S. Patent Publication No. 2012/0263701 and WO 2016/065301, which are both incorporated herein by reference in their entirety. In one embodiment, the XTEN is AE144. In another embodiment, the XTEN is AE288. In yet another embodiment, the XTEN is AE864.

In some embodiments, the half-life extending moiety comprises an Fc region. The Fc region comprises the hinge, CH2 and CH3 domains. In certain embodiments, the Fc region is from IgG1. In certain embodiments, the Fc region is from IgG2. In other embodiments, the Fc region is from IgG4. In yet other embodiments, the Fc region comprises the hinge and CH2 regions from IgG4 and the CH3 domain from IgG1. The hinge region from IgG4 can comprise the S228P mutation. The Fc region may also include one or more substitutions that reduce the effector function. In certain cases, the N-linked glycosylation site of the Fc region is mutated (e.g., T299A, T299C, N297Q). In certain cases, where the Fc region is from IgG2, the Fc region may comprise one or both of these mutations: V234A and G237A, that can reduce effector function. In other embodiments, the half-life extending moiety comprises two Fc regions fused by a linker. Exemplary heterologous moieties also include, e.g., FcRn binding moieties (e.g., complete Fc regions or portions thereof which bind to FcRn), single chain Fc regions (scFc regions, e.g., as described in U.S. Publ. No. 2008/0260738, and Intl. Publ. Nos. WO 2008/012543 and WO 2008/1439545), or processable scFc regions. In some embodiments, a heterologous moiety can include an attachment site for a non-polypeptide moiety such as polyethylene glycol (PEG), hydroxyethyl starch (HES), polysialic acid, or any derivatives, variants, or combinations of these moieties.

In some embodiments, the half-life extending moiety comprises human serum albumin (HSA) or a functional fragment thereof. Examples of albumin or the fragments or variants thereof are disclosed in US Pat. Publ. Nos. US2008/0194481, US2008/0004206, US2008/0161243, US2008/0261877, or US2008/0153751 or PCT Appl. Publ. Nos. WO2008/033413, WO2009/058322, or WO2007/021494, which are incorporated herein by reference in their entireties.

In certain instances, the half-life extending moiety can comprise an albumin binding moiety, which comprises an albumin binding peptide, a bacterial albumin binding domain, an albumin-binding antibody fragment, or any combinations thereof. For example, the albumin binding protein can be a bacterial albumin binding protein, an antibody or an antibody fragment including domain antibodies (see, e.g., U.S. Pat. No. 6,696,245). An albumin binding protein, for example, can be a bacterial albumin binding domain, such as the one of streptococcal protein G (Konig and Skerra (1998) J. Immunol. Methods 218, 73-83). Other examples of albumin binding peptides that can be used as conjugation partner are, for instance, those described in U.S. Pub. No. US2003/0069395 or Dennis et al. (2002) J. Biol. Chem. 277, 35035-35043. Domain 3 from streptococcal protein G, as disclosed by Kraulis et al., FEBS Lett., 378:190-194 (1996) and Linhult et al., Protein Sci., 11:206-213 (2002) is an example of a bacterial albumin-binding domain.

In certain embodiments, the half-life extending moiety can comprise one β subunit of the C-terminal peptide (CTP) of human chorionic gonadotropin or fragment, variant, or derivative thereof. The insertion of one or more CTP peptides into a recombinant protein is known to increase the in vivo half-life of that protein. See, e.g., U.S. Pat. No. 5,712,122 and U.S. Patent Appl. Publ. No. US 2009/0087411, incorporated by reference herein in their entirety.

In certain embodiments, the half-life extending moiety can comprise a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the polypeptides described herein. Non-limiting examples of PAS sequences are disclosed in US Pat. Publ. No. 2010/0292130 and PCT Appl. Publ. No. WO2008/155134 A1, incorporated by reference herein in their entirety.

In some embodiments, the half-life extending moiety is a soluble polymer including, but not limited to, polyethylene glycol (PEG), ethylene glycol/propylene glycol copolymers, carboxymethylcellulose, dextran, or polyvinyl alcohol. In one embodiment, the half-life extending moiety is PEG. The polyethylene glycol can have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa. In some embodiments, the polyethylene glycol can have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), each of which is incorporated herein by reference in its entirety.

### Stapled Peptides

In certain embodiments, one or more of the polypeptides described herein can be stabilized by peptide stapling (see, e.g., Walensky, J. Med. Chem., 57:6275-6288 (2014), the contents of which are incorporated by reference herein in its entirety). In certain embodiments, one or more of the polypeptides described herein can be stabilized by hydrocarbon stapling. In some embodiments, the stapled peptide (e.g., hydrocarbon stapled) is a polypeptide comprising or consisting of the prodomain fragment of GDF11 (e.g., 60-112, 60-114, or 60-117 of SEQ ID NO:1 or comprising 1 to 10 (e.g., 1, 2, 3, 4, 5, 6, 7, 8) amino acid substitutions, deletions and/or insertions therein). For example, the stapled peptide can include at least two (e.g., 2, 3, 4, 5, 6, 7, 8) amino acid substitutions, wherein the substituted amino acids are separated by two, three, or six amino acids, and wherein the substituted amino acids are non-natural amino acids with olefinic side chains.

“Peptide stapling” is a term coined from a synthetic methodology wherein two olefin-containing side-chains (e.g., cross-linkable side chains) present in a polypeptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). As used herein, the term “peptide stapling” includes the joining of two (e.g., at least one pair of) double bond-containing side-chains, triple bond-containing side-chains, or double bond-containing and triple bond-containing side chain, which may be present in a polypeptide chain, using any number of reaction conditions and/or catalysts to facilitate such a reaction, to provide a singly “stapled” polypeptide. The term “multiply stapled” polypeptides refers to those polypeptides containing more than one individual staple, and may contain two, three, or more independent staples of various spacings. Additionally, the term “peptide stitching,” as used herein, refers to multiple and tandem “stapling” events in a single polypeptide chain to provide a “stitched” (e.g., tandem or multiply stapled) polypeptide, in which two staples, for example, are linked to a common residue. Peptide stitching is disclosed, e.g., in WO 2008/121767 and WO 2010/068684, which are both hereby incorporated by reference in their entirety. In some instances, staples, as used herein, can retain the unsaturated bond or can be reduced. While many peptide staples have all hydrocarbon cross-links, other type of cross-links or staples can be used. For example, triazole-containing (e.g., 1, 4 triazole or 1, 5 triazole) crosslinks can be used (see, e.g., Kawamoto et al. 2012 Journal of Medicinal Chemistry 55:1137; WO 2010/060112). Stapling of a peptide using an all-hydrocarbon cross-link has been shown to help maintain its native conformation and/or secondary structure, particularly under physiologically relevant conditions (see, e.g., Schafmeister et al., J. Am. Chem. Soc., 122:5891-5892, 2000; Walensky et al., Science, 305:1466-1470, 2004).

Stapling the polypeptide(s) described herein by an all-hydrocarbon crosslink can improve stability and various pharmacokinetic properties.

The stapled polypeptide comprise at least two modified amino acids joined by an internal intramolecular cross-link (or “staple”), wherein the at least two amino acids are separated by 2, 3, or 6 amino acids. Stabilized peptides herein include stapled peptides, including peptides having two staples and/or stitched peptides. The at least two modified amino acids can be unnatural alpha-amino acids (including, but not limited to α,α-disubstituted and N-alkylated amino acids). There are many known unnatural amino acids any of which may be included in the peptides of the present invention. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and/para-substituted phenylalanines (e.g., substituted with —C(═O)C₆H₅; —CF₃; —CN; -halo; —NO2; CH₃), disubstituted phenylalanines, substituted tyrosines (e.g., further substituted with -Q=O)C₆H₅; —CF₃; —CN; -halo; —NO₂; CH₃), and statine.

In some embodiments, the disclosure features internally cross-linked (“stapled”) peptides comprising the amino acid sequence RELRLESIKSQILSKLRLKG (SEQ ID NO:51), wherein the side chains of two amino acids separated by two, three, or six amino acids are replaced by an internal staple; the side chains of three amino acids are replaced by an internal stitch; the side chains of four amino acids are replaced by two internal staples, or the side chains of five amino acids are replaced by the combination of an internal staple and an internal stitch. The stapled peptide can be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In certain embodiments, the stapled polypeptide comprises or consists of the amino acid sequence set forth in any one of SEQ ID NOs:28 to 32, wherein the side chains of two amino acids separated by two, three, or six amino acids are replaced by an internal staple; the side chains of three amino acids are replaced by an internal stitch; the side chains of four amino acids are replaced by two internal staples, or the side chains of five amino acids are replaced by the combination of an internal staple and an internal stitch.

In a particular embodiment, the stapled polypeptide comprises or consists of SPRELRLESIKSQILSKLRLKEAPNISREVVKQLLPKAPPLQQIL (SEQ ID NO:21), wherein the side chains of two amino acids separated by two, three, or six amino acids are replaced by an internal staple; the side chains of three amino acids are replaced by an internal stitch; the side chains of four amino acids are replaced by two internal staples, or the side chains of five amino acids are replaced by the combination of an internal staple and an internal stitch. Non-limiting examples of stapled peptides are:

(SEQ ID NO: 52)
SPRELRXESIXSQILSKLRLKEAPNISREVVKQLLPKAPPLQQIL
(SEQ ID NO: 53)
SPRELRLXSIKXQILSKLRLKEAPNISREVVKQLLPKAPPLQQIL
(SEQ ID NO: 54)
SPRELRLESIKSQILSKLRLXEAPXISREVVKQLLPKAPPLQQIL
(SEQ ID NO: 55)
SPRELRLESIKSQIXSKLRLKXAPNISREVVKQLLPKAPPLQQIL
(SEQ ID NO: 56)
SPRELRLESIKSQILSKLRLKEAPNISXEVVXQLLPKAPPLQQIL
(SEQ ID NO: 57)
SPRELRLESIKSQILSKLRLKEAPNISREVVKXLLPKAPXLQQIL,

wherein the X's in the above sequences can be the same or different non-natural amino acids which can be covalently joined (“stapled together”) using a ring-closing metathesis (RCM) reaction to form a cross-linked ring.

### Nucleic Acids Encoding the Polypeptides

This disclosure also encompasses nucleic acids encoding the polypeptides described herein. Using the amino acid sequences provided herein, one of ordinary skill in the art could easily identify and synthesize nucleic acid sequences encoding these polypeptides. The nucleic acid sequences may include a heterologous sequence or sequences (e.g., a regulatory element such as a promoter, enhancer, ribosome binding site, transcription terminator; a nucleic acid encoding a signal peptide).

### Methods of Making the Polypeptides

The polypeptides described herein can be made using methods well known in the art. For example, nucleic acids encoding the polypeptide(s) can be introduced into a host cell by a variety of known methods, e.g., transformation, transfection, electroporation, bombardment with nucleic acid-coated microprojectiles, etc. In some instances, the nucleic acids encoding the polypeptide(s) can be inserted into a vector appropriate for expression in the host cells before being introduced into the host cells. Typically, such vectors can contain sequence elements that enable expression of the inserted nucleic acids at the RNA and protein levels. Such expression vectors are well known in the art, and many are commercially available. In certain embodiments, the vector is a plasmid or a viral vector. The vectors can be introduced into host cells (e.g., microbial cells, yeast cells, insect cells, mammalian cells). Several kinds of host cells can be used including, e.g., bacterial cells such as Escherichia coli or Bacillus stearothermophilus, fungal cells such as Saccharomyces cerevisiae or Pichia pastoris, insect cells such as lepidopteran insect cells including Spodoptera frupperda cells, or mammalian cells such as Chinese hamster ovary (CHO) cells, baby hamster kidney (BHK) cells, monkey kidney cells, HeLa cells, human hepatocellular carcinoma cells, or 293 cells, among many others. In some cases, the host cell is a fungal cell. In a specific embodiment, the fungal cell is a filamentous fungal cell (e.g., Aspergillus niger, Aspergillus nidulans, Aspergillus oryzae, Trichoderma reesei). In other embodiments, the host cell is an algal cell, such as a microalgal cell (e.g., Chlamydomonas reinhardtii, Scenedesmus obliquus). The host cells containing the nucleic acids can be cultured under conditions so as to enable the cells to express the nucleic acids. The isolated polypeptide(s) can be formulated as a sterile composition for administration to a human subject. The formulation can have a pH of about 5.0 to about 6.5. In certain instances, the formulation has a pH of about 5.5.

### Methods of Use

The multimeric protein or polypeptide(s) described herein can be used to treat a neurodegenerative disease in a human subject in need thereof. In certain embodiments, the human subject has a disease or disorder of the central nervous system. In other embodiments, neurodegenerative disease in a human subject in need thereof. In certain embodiments, the human subject has a disease or disorder of the peripheral nervous system. The method comprises administering to the human subject a therapeutically effective amount of the protein or the pharmaceutical composition described herein. In certain instances, the neurodegenerative disease is Alzheimer's disease. In certain instances, the neurodegenerative disease is Parkinson's disease. In other instances, the neurodegenerative disease is Huntington's disease, amyotrophic lateral sclerosis, frontotemporal Dementia, Lewy Body Dementia, Mild Cognitive Impairment, Posterior Cortical Atrophy, Primary Progressive Aphasia, Progressive Supranuclear Palsy, or Vascular Dementia. In some embodiments, the neurodegenerative disease is spinal muscular atrophy (SMA), myasthenia gravis, Isaacs syndrome, Stiff-Person syndrome, Guillian-Barre syndrome, chronic inflammatory demyelinating polyneuropathy, amyotrophic lateral sclerosis, peripheral neuropathy, or thoracic outlet compression syndrome. In certain embodiments, the method involves administering a second agent that is also useful to treat the neurodegenerative disease. In certain embodiments, the second agent is an antibody or an antigen-binding fragment thereof. In certain embodiments, the antibody or antigen-binding fragment is anti-Abeta antibody or antigen-binding fragment (e.g., aducanumab, see, e.g., WO 2008/081008, incorporated by reference herein its entirety). In certain embodiments, the antibody or antigen-binding fragment is anti-α-synuclein antibody or antigen-binding fragment (see, e.g., WO 2010/069603 and WO 2012/177972, incorporated by reference herein their entirety). In other embodiments, the antibody or antigen-binding fragment is anti-tau antibody or antigen-binding fragment (see, e.g., WO 2014/100600 and WO 2012/049570, incorporated by reference herein their entirety). In yet other embodiments, the antibody or antigen-binding fragment is anti-TDP-43 antibody antigen-binding fragment (see, e.g., WO 2013/061163, incorporated by reference herein its entirety). In other embodiments, the antibody is an anti-LINGO-1 antibody (see, e.g., WO 2008/086006, incorporated by reference herein its entirety). In yet other embodiments, the antibody is an anti-TWEAK antibody (see, e.g., WO 2006/130374, incorporated by reference herein its entirety). In certain cases, the GDF11 polypeptide(s) is conjugated with or administered with a moiety or an agent that allows it traverse the blood brain barrier (e.g., FC5 single domain antibody, FC5-Fc, anti-transferrin antibody (e.g., OX26), insulin-like growth factor-1 receptor antibody, insulin receptor antibody).

In addition, the multimeric protein or polypeptide(s) described herein can be used to treat an age-related cardiac hypertrophy in a human subject in need thereof. The method comprises administering to the human subject a therapeutically effective amount of the protein or the pharmaceutical composition described herein. In certain instances the subject has or has been diagnosed with a condition selected from the group consisting of diastolic heart failure, cardiac hypertrophy, an age-related cardiac hypertrophy, hypertension, valvular disease, aortic stenosis, genetic hypertrophic cardiomyopathy, or stiffness of the heart due to aging.

The multimeric protein or polypeptide(s) described herein can also be used to treat a muscular or neuromuscular disease or disorder in a human subject in need thereof. The method comprises administering to the human subject a therapeutically effective amount of the protein or the pharmaceutical composition described herein. In certain instances, the muscular or neuromuscular disease or disorder is muscle atrophy, congestive obstructive pulmonary disease, muscle wasting syndrome, sarcopenia, or cachexia.

Also, the multimeric protein or polypeptide(s) described herein can be used to treat a metabolic disease or disorder resulting from abnormal glucose homeostasis. The method comprises administering to the human subject a therapeutically effective amount of the protein or the pharmaceutical composition described herein. In certain instances, the metabolic disease or disorder resulting from abnormal glucose homeostasis is type 2 diabetes, noninsulin-dependent diabetes mellitus, hyperglycemia, or obesity.

Furthermore, the multimeric protein or polypeptide(s) described herein can be used to treat a human subject suffering from a bone degenerative disorder. The method comprises administering to the human subject a therapeutically effective amount of the protein or the pharmaceutical composition described herein. In certain instances, the bone degenerative disorder is osteoporosis.

In addition, the multimeric protein or polypeptide(s) described herein can be used to diminish signs of aging. For example, the protein(s) described herein can be used to diminish dermatological signs of aging. The method comprises administering to the human subject (e.g., by topical application to the skin) a therapeutically effective amount of the protein or the pharmaceutical composition described herein.

Furthermore, the multimeric protein or polypeptide(s) described herein can be used to treat thymic insufficiency. For example, the protein(s) described herein can be used to induce growth of thymic tissues or thymic epithelial cells. The method comprises administering to the human subject a therapeutically effective amount of the protein or the pharmaceutical composition described herein.

The multimeric protein or polypeptide(s) described herein can be administered by any suitable method, e.g., intravenously, subcutaneously, intraperitoneally, intra-arterially, or intra-coronary arterially.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Materials & Methods

Expression of GDF11.

Expression plasmid pACE378 with CMV promoter and encoding the full length gene for human GDF11 followed by Ires-mDHFR for selection was engineered for expression in CHO cells. Suspension-adapted CHO cells were transfected with the plasmid and selected for integration by absence of nucleosides from serum-free media. Once established, this stable pool was cryopreserved. For production runs, cultures were expanded in serum-free media up to final volume of 20 L, grown for 4 days to high density (5×10⁶ cells/mL) with appropriate feeds, and shifted to a reduced temperature. Cultures were held at this reduced temperature for 11 days and then harvested by centrifugation and clarified through 0.2 micron 4 inch PolysepII Millipore cartridge filters.

Purification of GDF11.

17 L of clarified conditioned medium from the CHO cells expressing full length human GDF11 (ACE378) was concentrated to 4.5 L using a Millipore prepscale tangential flow filtration unit equipped with a 10K cellulose membrane. NaCl was added to 0.5 M and Na₂HPO₄ pH 7.0 to 20 mM, and the preparation was loaded onto a 220 mL Ni-Sepharose excel (GE Healthcare, Chalfont St. Giles, Buckinghamshire, UK) column at room temperature. The column was washed with 20 mM Na₂HPO₄ pH 7.0, 0.5 M NaCl, then with 5 mM NaH₂PO₄ pH 6.0, 100 mM NaCl, and step eluted with 30 mM, 300 mM, and 500 mM imidazole in 5 mM NaH₂PO₄ pH 6.0, 100 mM NaCl. Column fractions were analyzed for purity by SDS-PAGE. Fractions 26-42 from the 300 mM imidazole elution step (680 mL at 2.7 mg/mL, 1840 mg total protein) were pooled. NaCl and (NH₄)₂SO₄ were added to the Ni elution pool to final concentrations of 0.25 M NaCl and 1.2 M (NH₄)₂SO₄. The preparation was subjected to centrifugation at 12,000 rpm for 20 min and the clarified supernatant was loaded onto a 120 mL Butyl Sepharose (GE Healthcare) column at room temperature. The column was washed with 5 mM NaH₂PO₄ pH 6.0, 0.25 M NaCl, 1.2 M (NH₄)₂SO₄ and step eluted with 0.45 M and 0 M (NH₄)₂SO₄ in 5 mM NaH₂PO₄ pH 6.0, 0.25 M NaCl. Column fractions were analyzed for purity by SDS-PAGE. Fractions 4-7 from the 0.45 M (NH₄)₂SO₄ elution step (120 mL, 4.2 mg/mL, 500 mg) were pooled. The concentration of GDF11 was determined using an extinction coefficient of 1.27 for 1 mg/mL. The protein was dialyzed at 4° C. against 5 mM NaH₂PO₄ pH 6.0, 150 mM NaCl with 4×3.5 L changes of dialysis buffer, then filtered, aliquoted, and stored at −70° C.

Endoproteinase AspN/Furin Digestion of GDF11.

GDF11 (ACE378, 10 mg, 3.6 mg/mL) in 5 mM NaH₂PO₄, 150 mM NaCl, 50 mM Tris HCl pH 7.5 was treated for 2.5 hr at 37° C. with 10 of Endoproteinase AspN (Roche Diagnostics, Indianapolis, Ind.). NaCl was added to 0.5 M and the sample was loaded onto a 2 mL Ni-Sepharose excel column. The column was washed with 20 mM Na₂HPO₄ pH 7.0, 0.5 M NaCl, then with NaH₂PO₄ pH 6.0, 100 mM NaCl, and step eluted with 300 mM imidazole in 5 mM NaH₂PO₄ pH 6.0, 100 mM NaCl. Elution fractions were pooled and concentrated to ˜3 mL at 1.7 mg/mL. The protein was dialyzed at 4° C. against 5 mM NaH₂PO₄ pH 6.0, 150 mM NaCl with 2×1.8 L changes of dialysis buffer, aliquoted, and stored at −70° C. 4 mg of Asp-N digested huGDF11 was adjusted to 50 mM Tris HCl, pH 7.5 containing 1 mM CaCl₂. 72 μL of furin (Sigma F2677-50UN, >2 U/μL, St. Louis, Mo.) was added and the sample was incubated at 37° C. Aliquots were removed after 4 hr, 7 hr, and 24 hr for SDS-PAGE and activity measurements. After 24 hr, the remainder of the digest was aliquoted and stored at −70° C. EDTA was added to 5 mM at each time point to quench the furin reaction; and the soluble and precipitated fractions were separated by centrifugation at top speed for 4 min in an Eppendorf 5415C centrifuge.

Trypsin was obtained from Roche and human plasmin from Sigma. Digestions with trypsin were performed for 5 hr at 1:1000 enzyme:GDF11 ratio in 50 mM HEPES pH 8.2, 150 mM NaCl at room temperature and quenched with phenylmethanesulfonyl fluoride (PMSF, Sigma) and leupeptin (Sigma). Digestions with plasmin were performed for 2 hr at 1:300 enzyme:GDF11 ratio in 25 mM HEPES pH 7.5, 150 mM NaCl at room temperature and quenched with PMSF and leupeptin.

SDS-PAGE.

Samples were subjected to SDS-PAGE on a 4-20% gradient gel (Novex Life Technologies, Carlsbad, Calif.) under reducing and non-reducing conditions. The gels were stained with SimplyBlue™ SafeStain (Novex Life Technologies). Non-reduced samples were diluted with Laemmli non-reducing sample buffer, and heated at 75° C. for 5 min prior to analysis. Reduced samples were treated with sample buffer containing 2% 2-mercaptoethanol and heated at 95° C. for 4 min. GDF11 western blots of reduced SDS-PAGE samples were developed using anti-GDF8/11 C-terminal peptide goat polyclonal antibody sc6884 (C-20) from Santa Cruz Biotechnology (Dallas, Tex.).

Size Exclusion Chromatography.

Samples (100 μg for analytical and 500 μg for preparative analysis) were subjected to size exclusion chromatography (SEC) at room temperature on a GE Superdex 200 30/10 FPLC column in 10 mM sodium succinate, 75 mM NaCl, 100 mM L-arginine HCl pH 5.5 at a flow rate of 0.5 mL/min. The column effluent was monitored for absorbance at 280 nm. Molecular weight standards were run as controls and their chromatograms overlaid on the test sample chromatograms. For preparative runs, 1 mL fractions were collected. Fluorescently labeled samples (0.2-1 μg) were analyzed on a GE Superdex 200 5/150 GL column in the same buffer plus 1 mg/mL bovine serum albumin at a flow rate of 0.3 mL/min. The column effluent was monitored on an Agilent 1200 Series with Fluorescence Detector. The GDF11 samples were labeled with Alexa Fluor-488 (Invitrogen-Molecular Probes A10235) following the manufacturer's instructions.

Mass Spectrometry.

Samples were deglycosylated with PNGase F overnight at 37° C. 50 pmol of Asp-N-treated GDF11 and 50 pmol of SEC purified AspN/furin-treated GDF11 were reduced with 40 mM dithiothreitol (DTT) for 1 hr at 37° C. after deglycosylation. The reduced, deglycosylated samples and 75 pmol of the non-reduced, deglycosylated Asp-N-treated GDF11 were analyzed on an UPLC-LCT Premier mass spectrometer system, (Waters), using a BEH 1.7 μm 2.1×15 mm C₄ column (Waters) run at 0.07 mL/min for separation, with gradient (t=0 min 10% B, 10-50% B 0-40 min, 50-70% B 40-45 min, 70% B 45-50 min, 70%-10% B 50-55 min). Buffer A: water with 0.03% trifluoroacetic acid. Buffer B: acetonitrile with 0.024% trifluoroacetic acid. Molecular masses were generated by deconvolution using the MaxEnt 1 program. LC-MS/MS analysis was carried out using an UPLC-(Waters)-Obitrap-Elite/ETD mass spectrometer (Thermo Scientific) system. The separation was the same as described as above. For the CID experiment, the collision energy was set at 35% and the activation time at 30 MS.

Kinase Induced Receptor Activation (KIRA) Assay.

The Neuroscreen derivative of PC12 cells were plated at 2.2×10⁵ cells/mL per well in 24-well collage Type IV coated plates in Dulbecco's modified eagle medium (DMEM), heat-inactivated 10% Horse serum, 5% fetal bovine serum, 4 mM L-glutamine, and cultured overnight for 20 hr at 37° C. and 5% CO₂. The medium was dumped out and cells were washed with 1 mL/well phosphate-buffered saline (PBS). Test samples, 300 μL, were prepared containing 1:3 serial dilution of mature GDF11 (PeproTech, Rocky Hill, N.J.) reference standard, full length ACE378 huGDF11, and AspN-treated ACE378, all starting at 400 ng/mL. 250 μL of each sample was added to the wells and incubated for 1 hr at 37° C. and 5% CO₂. The sample cocktails were dumped out and the cells were washed with 1 mL of PBS. 300 μL of lysis buffer (10 mM Tris HCl, pH 8.0, 0.5% Nonidet-P40, 0.2% sodium deoxycholate, 50 mM NaF, 0.1 mM Na₃VO₄) was added, and after 15 min at room temperature, plates were frozen at −70° C. Samples were analyzed for pSMAD2/3 levels using the PathScan® Phospho-Smad2 (Ser465/467)/Smad3 (Ser423/425) sandwich Enzyme-linked Immunosorbent Assay (ELISA) assay kit from Cell Signaling Technology (#12001, Danvers, Mass.) following the manufacturer's protocol. The required number of microwells for each experiment was broken off after the microwell stripes reached room temperature. The 24-well plates were thawed at room temperature during the blocking period. Lysates were pipetted up and down with a multi-channel pipet 5 times to break up cell debris without creating bubbles. 260 μL of lysate was added to the blocked ELISA plates. Then 20 μL sample dilution buffer from kit was added and plates were shaken slowly for 2 hr at room temperature. Plates were washed 3-times with 10 mM Tris HCl pH 7.4, 150 mM NaCl, 0.05% Tween-20 (TBST). 100 μL of detection antibody was added and plates were shaken slowly for 1 hr at 37° C. Plates were washed 3-times with TBST. 100 μL of TMB buffer (Thermo Product #34028) was added and after 10 min, 100 μL 2 N H₂SO₄ was added to stop the reaction. Plates were read at 450 nm. Samples were analyzed in duplicate. Data were plotted with a 4-parameter curve fit and EC₅₀ values were determined from the curves.

Luciferase Reporter Assay on SMAD-Reporter Cells.

Neuroscreen SMAD2/3 reporter (Luciferase) cells were generated by transducing neuroscreen PC12 cells with lentivirus expressing the firefly luciferase gene under the control of a CMV promoter and SMAD transcriptional response element (TRE) using the Cignal Lenti SMAD reporter (luc) kit CLS-017 L from Qiagen (Germantown, Md.). After transduction, the neuroscreen cells were cultured under puromycin selection and once established, the reporter line was cryopreserved. To assess GDF11 function, the cells were plated at 2.0×10⁵ cells per well in 24-well collage Type IV coated plates in DMEM, heat-inactivate 10% horse serum, 5% fetal bovine serum, 5 μg/mL puromycin, and 100 ng/mL NGF, and cultured overnight for 20 hr at 37° C. and 5% CO₂. The medium was dumped out and cells were washed with 1 mL/well PBS. Serial 1:3 dilutions of test samples were prepared in 450 μL DMEM without serum with 100 ng/ml NGF. 250 μL of each sample was added to the wells and incubated for 5.5 hr at 37° C. and 5% CO₂. Sample cocktails were dumped out and the cells were washed with 1 mL of PBS. 100 μL of 1× lysis buffer from Promega was added and plates shaken for 15 min at room temperature. Lysates were pipetted up and down with a multi-channel pipet 5 times to break up cell debris. 20 μL of lysate and 100 μL/well substrate (Promega # E4550) were added to a black ELISA plate. After 1-2 min, luciferase activity was read on TR717 Microplate Lumimeter with WINGLOW software. Samples were analyzed in duplicate. Data were plotted with a 4-parameter curve fit and EC₅₀ values were determined from the curves. The time dependence of luciferase expression was assessed and maximal activity was detected after 6 hr. No GDF11-induced luciferase activity was detected after 1.5 or 3 hr, consistent with the need for transcription of the luciferase gene.

Biotinylation of GDF11.

GDF11 (2.9 mg/mL) in 15 mM sodium succinate pH 5.5, 150 mM NaCl was incubated in the dark with 2 mM sodium meta-Periodate (Thermo Scientific) for 30 min at room temperature and immediately desalted on a Zeba spin desalting column equilibrated in 5 mM NaH₂PO₄ pH 6.0, 150 mM NaCl (Thermo Scientific). EZ-LinkHydrazide-LC-Biotin (Thermo Scientific) was added to a final concentration of 5 mM from a 50 mM stock prepared in dimethylsulfoxide. HEPES pH 7.2 was added to 50 mM and the sample was incubated in the dark for 2 hr at room temperature and then desalted on a Zeba spin desalting column equilibrated in 5 mM NaH₂PO₄ pH 6.0, 150 mM NaCl. 12 mg of biotinylated GDF11 (in 5 mM NaH₂PO₄, 150 mM NaCl, 50 mM Tris HCl pH 7.5 was treated for 2.5 hr at 37° C. with 6 μg of Endoproteinase AspN then purified on Ni-Sepharose excel column as described above for the non-biotinylated sample. The protein was dialyzed at 4° C. against 5 mM NaH₂PO₄ pH 6.0, 150 mM NaCl with 2, 1 L changes of dialysis buffer, aliquoted, and stored at −70° C. To produce biotinylated GDF11 peptide 60-112/114/-mature GDF11 complex, an aliquot of the sample was digested with furin as described above. To produce free biotinylated GDF11 peptide 60-112/114, an aliquot of the biotinylated AspN-treated GDF11 was heated at 95° C. for 10 min and centrifuged at top speed for 4 min in an Eppendorf centrifuge. Following this treatment, GDF11 peptide 60-112/114 was in the supernatant and the rest of the protein precipitated and was in the pellet fraction. The specificity of labeling of GDF11 with biotin hydrazide was confirmed by Western blotting using Streptavidin Horseradish peroxidase for detection. Only full length GDF11 and glycopeptide 60-114 were detected and not the major fragments 122-407 or 299-407.

Octet Binding Studies.

Binding characteristics of the biotinylated samples were evaluated by Octet on an Octet RED System (FortéBio™, Menlo Park, Calif.) using Dip and Read™ Streptavidin (SA) Biosensors (FortéBio™). Samples were prepared in 5 mM NaH₂PO₄ pH 6.0, 150 mM NaCl, 0.005% Tween-20. Loading and dissociation measurements were performed in the same buffer. The ability of biotinylated AspN/furin GDF11 to bind Type I and II receptors was also assessed by Octet using the same loading conditions, followed by treatment of the receptors at 10 μg/mL. Recombinant Human Activin RIB (ALK-4)-Fc chimeric protein and recombinant Human Activin RIIA-Fc chimeric protein were obtained from R&D systems and reconstituted at 200 μg/mL. The streptavidin tips were presoaked in Octet buffer for 15 min. The tips were loaded into the instrument, washed for 1 min with the buffer, then biotinylated samples and controls were loaded for 5 min. The tips were washed for 1 min and dissociation monitored for 30 min in the presence of 20 μM free biotin. For secondary binding studies with the receptors, the tips were loaded with biotinylated GDF11 and controls, and washed as described above, then the receptor samples were loaded for 15 min and dissociation monitored for 5 min.

Computational Model of the GDF11 Complex.

A model for the N-terminal prodomain peptide-mature GDF11 complex was produced as follows: Using the crystal structure of the latent procomplex 3RJR.pdb (Shi et al., Nature, 474:343-349 (2011)) from the Protein Data Bank (PDB, Berman et al., Nucl. Acids Res. 28:235-242 (2000)), the mature domain of TGF-β1 was superposed with that of GDF11 (5E4G.pdb, Padyana et al., Acta Crystallogr. F Struct. Biol. Commun., 72:160-164 (2016)) using PyMOL (PyMOL, 2012). Because the structure of GDF11 contains a monomer, symmetry mates were used in PyMOL to reconstitute the dimer with the correct biological interface. The side chain residues of the GDF11 α2 helix were adapted to likely equivalent positions on the backbone of the TGFβ1 α2 helix. Then, the TGFβ1 position of the α2 helix was used as an initial position to place the GDF11 α2 helix relative to the mature domain of GDF11. The GDF11 α1 helix was docked relative to the GDF11 mature domain dimer in the active conformation (Padyana et al., (supra)) using the protein-protein docking software PIPER (Kozakov et al., Biophysical Journal 89(2):867-875 (2005); Kozakov et al., Proteins: Structure, Function, and Bioinformatics, 65(2):392-406 (2006)). We emphasize the active conformation because the wrist helices of GDF11 or TGFβ in the active and signaling dimer conformation conflict with the location of the prodomain α1 helix in the latent conformation. We infer that the GDF11 α1 helix of the remaining prodomain peptide must be located outside of the active mature domain dimer interface. For docking the putative GDF11 α1 helix, we used only the C-terminal region of the TGFβ1 α1 helix as a template and adapted the sequence to the equivalent GDF11 residues (SRELRLESIKSQILSKLRL (SEQ ID NO:22)) because the al helices of human GDF11 and porcine TGFβ1 are 58% identical and secondary structure predictions indicate strong helicity in that region. In this model, we assume that the dislocated GDF11 α1 helix remains helical. In PIPER, docked poses that are similar are clustered together. For the model, we selected docked poses from the two largest clusters (each combining 453 and 435 poses, respectively), which clustered in approximately the same positions on either side of the mature GDF11 dimer. The lasso linker between the α1 and α2 helices was modeled using Molecular Operating Environment (MOE, Chemical Computing Group, 2016) and the Amber10 force field (Cornell et al., J. Am. Chem. Soc., 117(19):5179-5197 (1995)). Lastly, the modeled peptide structure was energy-minimized with MOE/Amber10, allowing the peptide coordinates to converge into local minima while the atom coordinates of the mature domain structure were tethered close to their original positions. Shi et al. (2011) postulated that despite low sequence identities, insertions, and deletions, the α2 helix is conserved across all TGF-β superfamily members, and the C-terminal portion of the al helices have preserved amphipathic sequence signatures for many family members, including myostatin and GDF11. Four out of nine secondary structure prediction methods we tested predict helical content in the lasso region of GDF11, suggesting an additional helix between the α1 and α2 helices. These prediction methods also revealed that N-terminal sequences in the GDF11 prodomain contain a putative transmembrane helix-like 13-Ala stretch from Ala-29 to Ala-41. Thus, the GDF11 prodomain residues 25-122 may form up to four helices, as compared to two in TGF-β1. To facilitate comparisons with other TGF-β family members, we have retained the α1 and α2 helix designations that were used by Shi et al. (2011) for GDF11.

Reduction-Oxidation Dimerization Studies.

Eleven GDF11 constructs containing truncated prodomains fused to the mature domain of GDF11 were engineered and expressed as transients in CHO cells using similar growth conditions described for production of ACE378. Table 1 lists the designs for all the constructs.

TABLE 1

| Construct | Protein sequence attributes | SEQ ID NO |
|---|---|---|
| pACE487 | 8xHis-TEV-GDF (G61-D122)-G-4xG4S-GDF (N299-S407) | 5 |
| pACE490 | 8xHis-TEV-GDF (S71-A123)-3xG4S-GDF (N299-S407) | 6 |
| pACE491 | 8xHis-TEV-GDF (G61-L114)-3xG4S-GDF (N299-S407) | 7 |
| pACE492 | 8xHis-TEV-GDF (D60-L114)-3xG4S-GDF (N299-S407) | 8 |
| pACE493 | 8xHis-TEV-GDF (D60-L114)-4xG4S-GDF (N299-S407) | 9 |
| pACE494 | 8xHis-TEV-GDF (N299-S407)-2xG4S-GDF (S71-L114) | 10 |
| pACE495 | 8xHis-TEV-GDF (N299-S407)-3xG4S-GDF (S71-L114) | 11 |
| pACE496 | 8xHis-TEV-GDF (N299-S407)-3xG4S-GDF (D60-L114) | 12 |
| pACE497 | 8xHis-TEV-GDF (N299-S407)-3xG4S-SP-GDF (R72-L114) | 13 |
| pACE498 | 8xHis-TEV-GDF (D60-D122)-2xG4S-GDF (N299-S407) | 14 |
| pACE499 | 8xHis-TEV-GDF (N299-S407)-3xG4S-GDF (S71-D122) | 15 |
| pACE382 | GDF (N299-S407) | 20 |

A series of 11 prodomain-mature fusion constructs were engineered and expressed in CHO cells. All contained the GDF11 signal peptide linked to an 8 histidine affinity tag (SEQ ID NO: 3) with a TEV protease cleavage N-terminal to the designs. G4S (SEQ ID NO: 4) spacer sequences of varying lengths were included in the designs. Pro sequences are underlined and mature domain sequences are boldened.

The amino acid sequence of the protein encoded by the above-referenced constructs is provided below. The GDF11 signal peptide is in italics; the TEV protease cleavage sequence is boldened; the first GDF11 sequence is underlined; and the second GDF11 sequence is both underlined and boldened.

pACE487 (SigPep\hat{ }8xHis-TEV-GDF (G61-D122)-G-4xG4S-
GDF11 (N299-S407)):
(SEQ ID NO: 5)
metdtlllwvlllwvpgahaHHHHHHHHENLYFQSGCPVCVWRQHSRELR
LESIKSQILSKLRLKEAPNISREVVKQLLPKAPPLQQILDLHDFQGDGGG
GGSGGGGSGGGGSGGGGSNLGLDCDEHSSESRCCRYPLTVDFEAFGWDWI
IAPKRYKANYCSGQCEYMFMQKYPHTHLVQQANPRGSAGPCCTPTKMSPI
NMLYFNDKQQIIYGKIPGMVVDRCGCS
pACE490 (SigPep\hat{ }8xHis-TEV-GDF (S71-A123)-3xG4S-
GDF (N299-S407)):
(SEQ ID NO: 6)
metdtlllwvlllwvpgahaHHHHHHHHENLYFQSRELRLESIKSQILSK
LRLKEAPNISREVVKQLLPKAPPLQQILDLHDFQGDAGGGGSGGGGSGGG
GSNLGLDCDEHSSESRCCRYPLTVDFEAFGWDWIIAPKRYKANYCSGQCE
YMFMQKYPHTHLVQQANPRGSAGPCCTPTKMSPINMLYFNDKQQIIYGKI
PGMVVDRCGCS
pACE491 (SigPep\hat{ }8xHis-TEV-GDF (G61-L114)-3xG4S-GDF
(N299-S407)):
(SEQ ID NO: 7)
metdtlllwvlllwvpgahaHHHHHHHHENLYFQGCPVCVWRQHSRELRL
ESIKSQILSKLRLKEAPNISREVVKQLLPKAPPLQQILGGGGSGGGGSGG
GGSNLGLDCDEHSSESRCCRYPLTVDFEAFGWDWIIAPKRYKANYCSGQC
EYMFMQKYPHTHLVQQANPRGSAGPCCTPTKMSPINMLYFNDKQQIIYGK
IPGMVVDRCGCS
pACE492 (SigPep\hat{ }8xHis-TEV-GDF (D60-L114)-3xG4S-GDF
(N299-S407)):
(SEQ ID NO: 8)
metdtlllwvlllwvpgahaHHHHHHHHENLYFQSDGCPVCVWRQHSREL
RLESIKSQILSKLRLKEAPNISREVVKQLLPKAPPLQQILGGGGSGGGGS
GGGGSNLGLDCDEHSSESRCCRYPLTVDFEAFGWDWIIAPKRYKANYCSG
QCEYMFMQKYPHTHLVQQANPRGSAGPCCTPTKMSPINMLYFNDKQQIIY
GKIPGMVVDRCGCS
pACE493 (SigPep\hat{ }8xHis-TEV-GDF (D60-L114)-4xG4S-GDF
(N299-S407)):
(SEQ ID NO: 9)
metdtlllwvlllwvpgahaHHHHHHHHENLYFQSDGCPVCVWRQHSRE
LRLESIKSQILSKLRLKEAPNISREVVKQLLPKAPPLQQILGGGGSGGG
GSGGGGSGGGGSNLGLDCDEHSSESRCCRYPLTVDFEAFGWDWIIAPKR
YKANYCSGQCEYMFMQKYPHTHLVQQANPRGSAGPCCTPTKMSPINMLY
FNDKQQIIYGKIPGMVVDRCGCS
pACE494 (SigPep\hat{ }8xHis-TEV-GDF (N299-S407)-2xG4S-
GDF (S71-L114)):
(SEQ ID NO: 10)
metdtlllwvlllwvpgahaHHHHHHHHENLYFQSNLGLDCDEHSSES
RCCRYPLTVDFEAFGWDWIIAPKRYKANYCSGQCEYMFMQKYPHTHLV
QQANPRGSAGPCCTPTKMSPINMLYFNDKQQIIYGKIPGMVVDRCGCS
GGGGSGGGGSRELRLESIKSQILSKLRLKEAPNISREVVKQLLPKAPP
LQQIL
pACE495 (SigPep\hat{ }8xHis-TEV-GDF (N299-S407)-3xG4S-
GDF (S71-L114)):
(SEQ ID NO: 11)
metdtlllwvlllwvpgahaHHHHHHHHENLYFQSNLGLDCDEHSSES
RCCRYPLTVDFEAFGWDWIIAPKRYKANYCSGQCEYMFMQKYPHTHLV
QQANPRGSAGPCCTPTKMSPINMLYFNDKQQIIYGKIPGMVVDRCGCS
GGGGSGGGGSGGGGSSRELRLESIKSQILSKLRLKEAPNISREVVKQL
LPKAPPLQQIL
pACE496 (SigPep\hat{ }8xHis-TEV-GDF (N299-S407)-3xG4S-
GDF (D60-L114)):
(SEQ ID NO: 12)
metdtlllwvlllwvpgahaHHHHHHHHENLYFQSNLGLDCDEHSSES
RCCRYPLTVDFEAFGWDWIIAPKRYKANYCSGQCEYMFMQKYPHTHLV
QQANPRGSAGPCCTPTKMSPINMLYFNDKQQIIYGKIPGMVVDRCGCS
GGGGSGGGGSGGGGSDGCPVCVWRQHSRELRLESIKSQILSKLRLKEA
PNISREVVKQLLPKAPPLQQIL
pACE497 (SigPep\hat{ }8xHis-TEV-GDF (N299-S407)-3xG4S-
SP-GDF (R72-L114)):
(SEQ ID NO: 13)
metdtlllwvlllwvpgahaHHHHHHHHENLYFQSNLGLDCDEHSSESRC
CRYPLTVDFEAFGWDWIIAPKRYKANYCSGQCEYMFMQKYPHTHLVQQAN
PRGSAGPCCTPTKMSPINMLYFNDKQQIIYGKIPGMVVDRCGCSGGGGSG
GGGSGGGGSSPRELRLESIKSQILSKLRLKEAPNISREVVKQLLPKAPPL
QQIL
pACE498 (SigPep\hat{ }8xHis-TEV-GDF (D60-D122)-2xG4S-GDF
(N299-S407)):
(SEQ ID NO: 14)
metdtlllwvlllwvpgahaHHHHHHHHENLYFQSDGCPVCVWRQHSREL
RLESIKSQILSKLRLKEAPNISREVVKQLLPKAPPLQQILDLHDFQGDGG
GGSGGGGSNLGLDCDEHSSESRCCRYPLTVDFEAFGWDWIIAPKRYKANY
CSGQCEYMFMQKYPHTHLVQQANPRGSAGPCCTPTKMSPINMLYFNDKQQ
IIYGKIPGMVVDRCGCS
pACE499 (SigPep\hat{ }8xHis-TEV-GDF (N299-S407)-3xG4S-
GDF (S71-D122)):
(SEQ ID NO: 15)
metdtlllwvlllwvpgahaHHHHHHHHENLYFQSNLGLDCDEHSSESRC
CRYPLTVDFEAFGWDWIIAPKRYKANYCSGQCEYMFMQKYPHTHLVQQAN
PRGSAGPCCTPTKMSPINMLYFNDKQQIIYGKIPGMVVDRCGCSGGGGSG
GGGSGGGGSSRELRLESIKSQILSKLRLKEAPNISREVVKQLLPKAPPLQ
QILDLHDFQGD

The nucleic acid sequences of two of the above constructs are also provided below:

PACE490
(SEQ ID NO: 16)
ATGGAGACAGACACACTCCTGCTGTGGGTACTGCTGCTCTGGGTTCCAGG
AGCTCACGCTCATCATCATCACCATCACCATCATGAGAACCTGTACTTCC
AGAGCCGAGAGCTGAGACTTGAGAGCATCAAGTCTCAGATCCTGAGCAAA
CTGCGGCTCAAGGAGGCTCCCAACATCAGTCGCGAGGTGGTGAAGCAGTT
GCTGCCCAAGGCTCCTCCACTGCAACAGATCTTGGACCTACACGACTTCC
AGGGTGACGCTGGAGGTGGAGGTTCTGGCGGTGGAGGATCCGGTGGAGGT
GGATCTAACCTGGGTCTGGACTGCGACGAGCACTCAAGCGAGTCCCGCTG
CTGTAGGTATCCTCTCACAGTGGACTTTGAGGCTTTCGGCTGGGACTGGA
TCATCGCACCTAAGCGCTACAAGGCCAACTACTGCTCCGGCCAGTGCGAG
TACATGTTCATGCAGAAGTACCCCCATACCCATTTGGTGCAGCAGGCCAA
TCCAAGAGGCTCTGCTGGACCCTGTTGTACCCCTACCAAGATGTCCCCAA
TCAACATGCTCTACTTCAACGACAAGCAGCAGATCATCTACGGCAAGATC
CCTGGCATGGTGGTGGATCGCTGTGGCTGCTCTTGA
PACE498
(SEQ ID NO: 17)
ATGGAGACAGACACACTCCTGCTGTGGGTACTGCTGCTCTGGGTTCCAGG
AGCTCACGCTCATCATCATCACCATCACCATCATGAGAACCTGTACTTCC
AGAGCGATGGTTGCCCTGTGTGCGTTTGGCGACAGCACAGCCGAGAGCTG
AGACTTGAGAGCATCAAGTCTCAGATCCTGAGCAAACTGCGGCTCAAGGA
GGCTCCCAACATCAGTCGCGAGGTGGTGAAGCAGTTGCTGCCCAAGGCTC
CTCCACTGCAACAGATCTTGGACCTACACGACTTCCAGGGTGACGGCGGT
GGAGGATCCGGTGGAGGTGGATCTAACCTGGGTCTGGACTGCGACGAGCA
CTCAAGCGAGTCCCGCTGCTGTAGGTATCCTCTCACAGTGGACTTTGAGG
CTTTCGGCTGGGACTGGATCATCGCACCTAAGCGCTACAAGGCCAACTAC
TGCTCCGGCCAGTGCGAGTACATGTTCATGCAGAAGTACCCCCATACCCA
TTTGGTGCAGCAGGCCAATCCAAGAGGCTCTGCTGGACCCTGTTGTACCC
CTACCAAGATGTCCCCAATCAACATGCTCTACTTCAACGACAAGCAGCAG
ATCATCTACGGCAAGATCCCTGGCATGGTGGTGGATCGCTGTGGCTGCTC
TTGA

All the constructs of Table 1 were expressed in 300 mL cultures. ACE490 and ACE498 were purified on 10 mL Ni-Sepharose Excel columns using the same wash and steps described for ACE378, then aliquoted, and stored at −70° C. For reduction of ACE490 and ACE498, samples were first concentrated to ˜1.5 mg/mL in Amicon Ultra-4 centrifugal filter units (10K cellulose membranes), then treated with 3 mM DTT for 30 min at room temperature and desalted into 25 mM Na₂HPO₄ pH 7.0, 100 mM NaCl on Zeba spin desalting columns (Thermo Scientific). Half of the reduced protein was treated with 1 mM Aldrithiol™ (AT, Sigma-Aldrich) for 30 min at room temperature and again desalted into 25 mM Na₂HPO₄ pH 7.0, 100 mM NaCl on Zeba spin desalting columns. To induce dimer formation, equal amounts of the DTT only and DTT/AT treated samples were mixed and incubated for 20 hr at room temperature or incubated in the presence of 1M guanidine HCl for 70 hr at 4° C. Extent of dimer formation was assessed by SDS-PAGE. A portion of the DTT/AT-treated ACE498 preparation was further treated with endoproteinase AspN at a 1:1000 protein:enzyme ratio for 60 min at 37° C.

### Example 2: Proteolytic Activation of GDF11

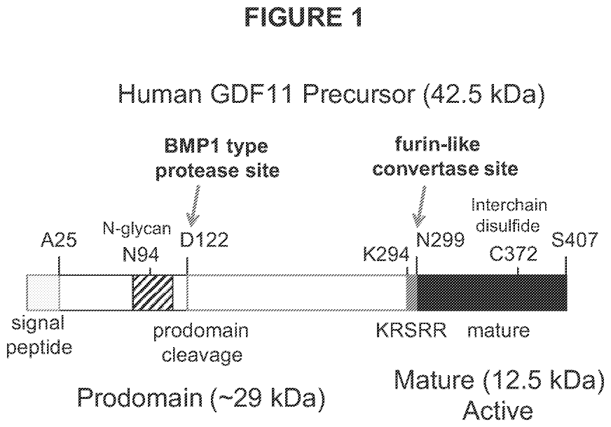

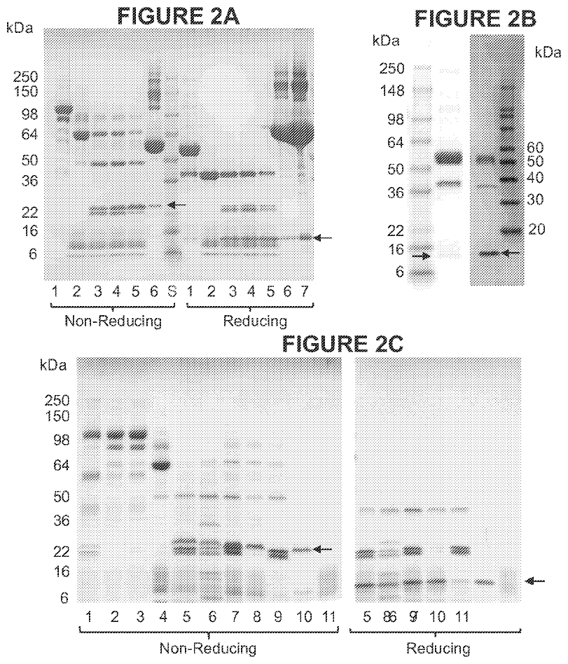

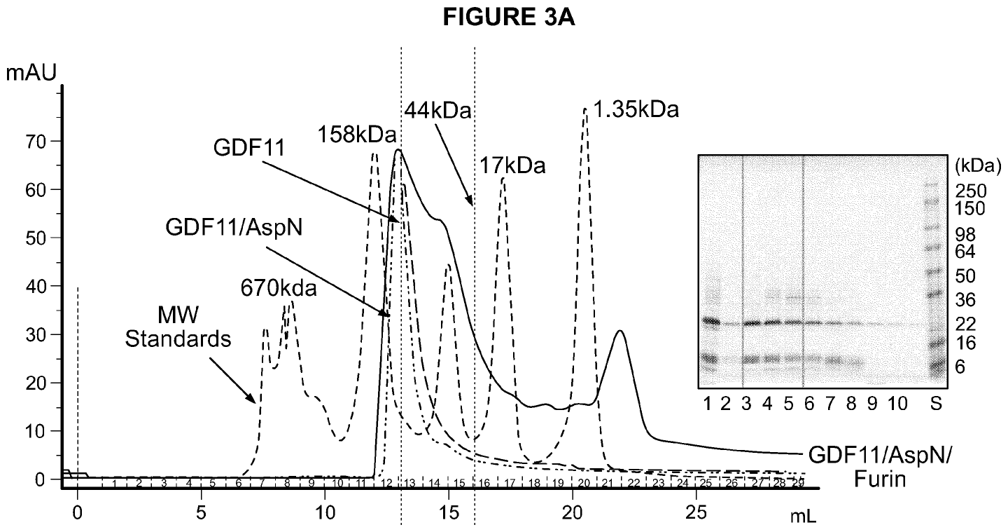

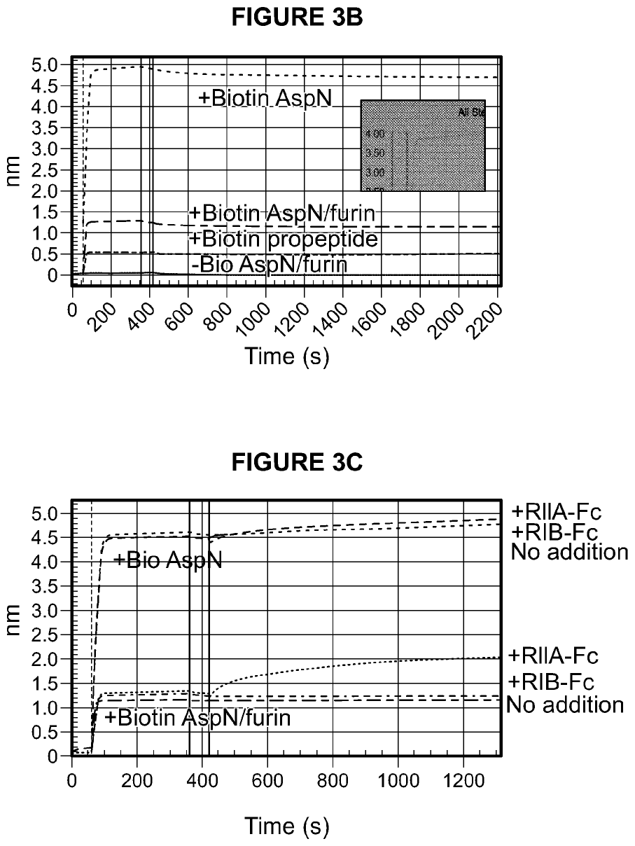

A construct encoding full-length human GDF11 was expressed in CHO cells and purified from the culture medium by sequential column chromatography steps on Ni Excel and Butyl Sepharose. SDS-PAGE analysis of the purified product (FIG. 2A, lane 1) revealed a single prominent band with molecular mass of 110 kDa under non-reducing and 55 kDa under reducing conditions consistent with the molecular mass of the GDF11 precursor containing both the pro and mature domains and formation of the characteristic homodimer. The 55 kDa band was immunoreactive with anti-GDF11 antibody specific for mature GDF11 when analyzed by SDS-PAGE/Western blotting under reducing conditions (FIG. 2B). The apparent purity of the full length GDF11 was approximately 90%. A 90 kDa band (non-reducing) and 40 kDa (reducing) that represented ˜5% of the total protein and several minor bands of less intensity of varying molecular weights were also observed. The reduced 40 kDa band also reacted with the anti-GDF11 antibody by Western blot analysis (FIG. 2B). Mass spectrometry revealed that the 40 kDa band contained GDF11 residues 122-407, and thus was produced by cleavage at the BMP1 site (FIG. 1). By size exclusion chromatography (SEC), the full length GDF11 migrated as a single homogeneous peak with apparent molecular weight of 100 kDa (FIG. 3A). No aggregates were detected. From a 20 L culture, ˜500 mg of the purified protein was recovered.

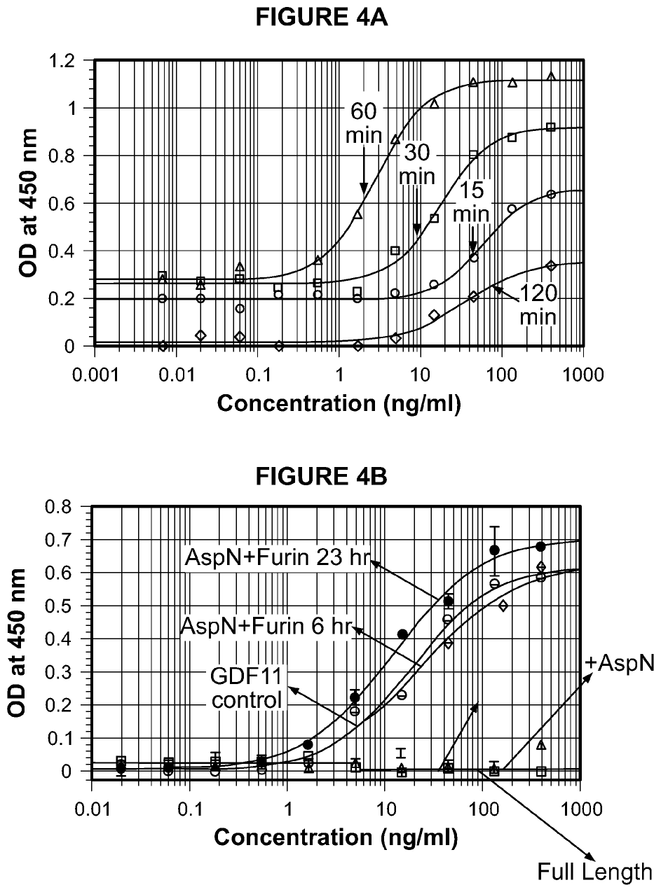

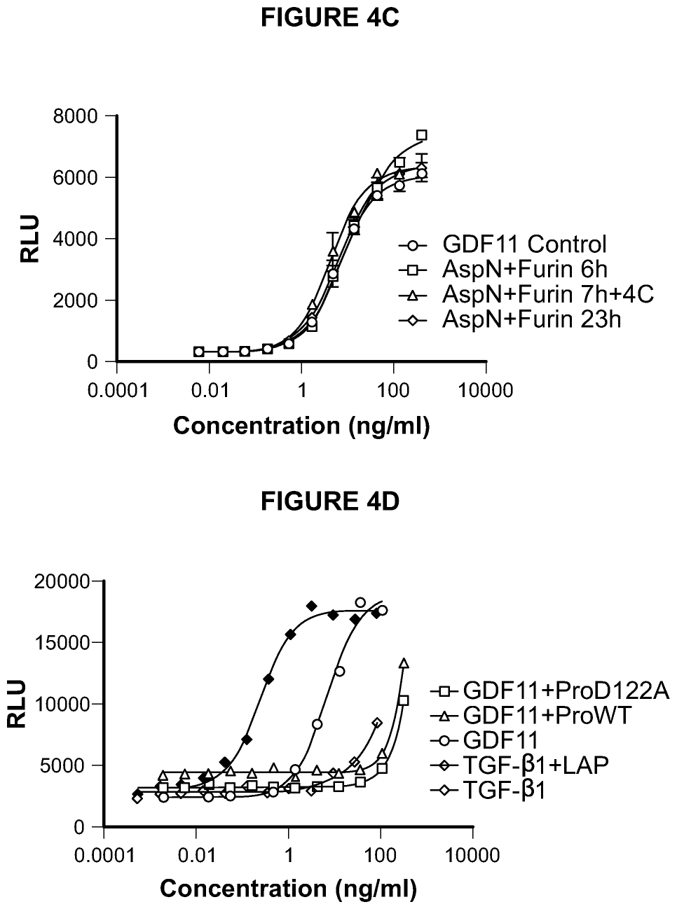

GDF11 was tested for function in a Kinase induced receptor activation (KIRA) assay on PC12 cells monitoring SMAD 2/3 phosphorylation. FIG. 4A shows a time course of GDF11-induced SMAD 2/3 phosphorylation. The maximum efficacious response occurred at 60 min. Comparison of the activity of mature GDF11 standard versus the recombinant full length protein (FIG. 4B) revealed that the GDF11 standard was a potent activator of SMAD 2/3 phosphorylation with an EC50 of 20 ng/mL (˜1 nM), but recombinant full length GDF11 was inactive, consistent with the need for proteolytic activation (FIG. 1). In an attempt to activate the precursor, the sample was treated with furin, but was unable to promote cleavage as assessed by SDS-PAGE (FIG. 2C, lane 3). In subsequent studies it was discovered that if the protein was first treated with endoproteinase AspN to cleave at Asp-122, the GDF11 then became susceptible to cleavage at the furin site (see below). FIG. 2A, lane 2 shows an SDS-PAGE analysis of GDF11 that had been treated with endoproteinase AspN for 2.5 hr at 37° C. A single major cleavage product with molecular weights of 65 kDa under non-reducing and 40 kDa under reducing conditions was detected, with near quantitative cleavage. A diffuse low molecular weight band ranging in size from 6-16 kDa was also detected that showed a similar electrophoretic pattern under reducing and non-reducing conditions. No additional cleavage products were generated with 10-fold higher concentrations of the enzyme under the same conditions or following longer incubation.

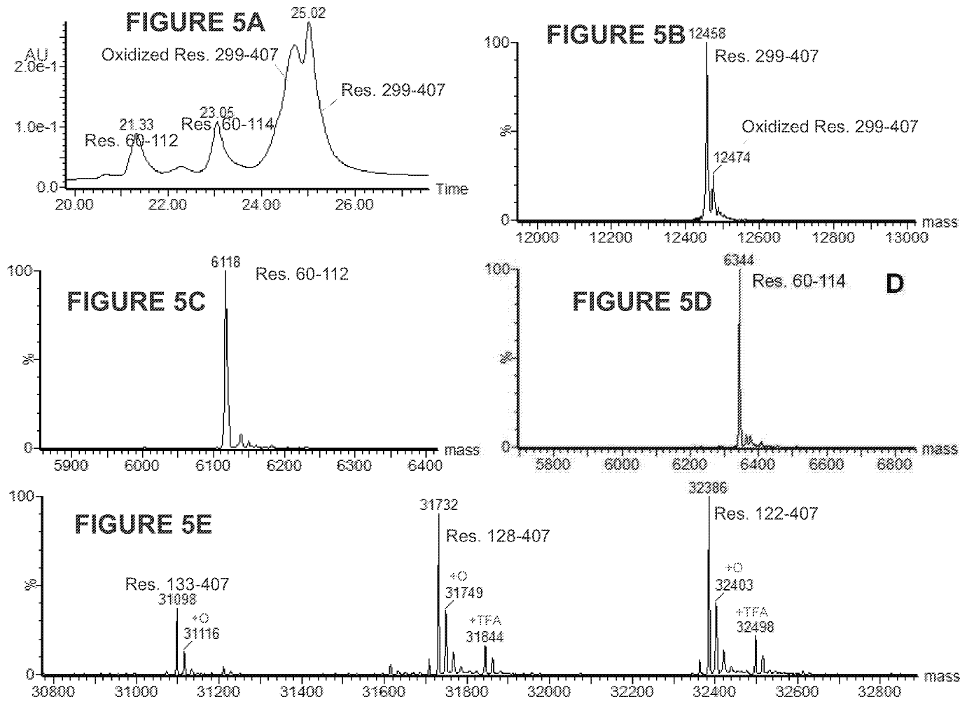

Extensive analysis of the AspN alone treated GDF11 by mass spectrometry (FIG. 5) revealed that the major cleavage product corresponded to residues 122-407 (predicted mass 32383.1 Da, observed mass 32386 Da) and glycopeptide residues 60-114 (predicted deglycosylated mass 6344.5 Da, observed mass 6344 Da). In addition, fragments corresponding to residues 128-407 (predicted mass 31729.4 Da, observed mass 31732 Da), residues 133-407 (predicted mass 31095.7 Da, observed mass 31098 Da) and residues 60-117 (predicted deglycosylated mass 6710.0 Da, observed mass 6708 Da) were detected. In total under the limiting conditions used for digestion with endoproteinase AspN, only 5 of 17 aspartic acids were digested and all were in or near the N-terminal glycopeptide. None of the 11 downstream sites including 6 in the mature domain were targeted. GDF11 contains a single N-linked glycosylation site at Asn-94. From mass spectrometry analysis of the sample without deglycosylation, various glycoforms of the residue 60-114/117 glycopeptide were detected ranging in mass from 7951 Da to 8769 Da (residues 60-117, 6709 Da without glycan along with a small amount of glycoforms of residue 25-114/117 glycopeptide ranging in mass from 9958-12369 Da). These glycoforms account for the diffuse 6-16 kDa banding pattern seen by SDS-PAGE (FIG. 2A, lane 2). The MS data also revealed an intrachain Cys 62-Cys-65 disulfide. In TGF-β1, Cys4, corresponding to Cys-62 in GDF11, is disulfide linked to latent TGF-beta binding protein LTBP (Shi et al., Nature, 474:343-9 (2011)). This association tethers TGF-β1 to the extracellular matrix, where it plays a critical role in local activation of TGF-β. For TGF-β1, there is no second intrachain Cys to pair with that corresponds to Cys-65 in GDF11. MS also provided confirmation of the processing site of the signal peptide, leading to the predicted N-terminus of Ala-25, and identified two O-linked glycosylation sites at Ser-49 (>75% occupied) and Ser-54 (99% occupied). Like the full length protein, AspN-treated GDF11 migrated by SEC as a single peak of molecular weight 100 kDa (FIG. 3A) with no detectable aggregates in the preparation. No free low molecular weight components were detected by SEC despite the presence of the 60-114 glycopeptide in the SDS-PAGE and MS analyses (FIG. 2A, lane 2, FIG. 5), indicating that the fragment is non-covalently associated with the rest of the protein.

Furin treatment of the AspN digestion product led to further processing of the GDF11. 80% of the residue 122-407 AspN fragment was digested by furin after 6 hr, and >95% was digested after 23 hr (FIG. 2A, lanes 3 and 5). Three new major cleavage products were produced with molecular weights of 45, 25, and 23 kDa under non-reducing conditions, and 40, 23/22, and 14 kDa under reducing conditions. The broad low molecular weight 6-16 kDa band seen in the absence of furin treatment was retained in the double digest. Mature GDF11 exists as a disulfide-linked homodimer with apparent molecular weights of 25 kDa and 14 kDa under non-reducing and reducing conditions, respectively. To aid in the identification of bands corresponding to mature GDF11 a preparation of mature GDF11 standard was included in the analysis (lanes 6 and 7, arrows denote the position of the mature GDF11 under reducing and non-reducing conditions). The identity of the fragment was confirmed by mass spectrometry (FIG. 5, predicted mass for mature GDF11 amino acid residues 299-407 12457.4 Da, observed mass 12458 Da). Following 23 hr of digestion, mature GDF11 was the major cleavage product, present at about 60% of the theoretical yield. The AspN/furin cleavage product was tested for function in PC12 cells in the SMAD 2/3 phosphorylation assay as well as in a SMAD reporter luciferase assay in which luciferase expression is under control of the SMAD transcriptional response element (TRE) (FIGS. 4B and 4C). Both assays confirmed proteolytic activation of the GDF11 precursor. The potency of the processed product (EC50=˜1 nM) was identical to that of the GDF11 standard. No activation occurred when full length GDF11 that was treated with endoproteinase AspN alone was analyzed (FIG. 4B).

Together these studies revealed that the proteolytic activation of full length GDF11 in vitro required two steps; first cleavage at Asp-122 to access the furin cleavage site, and then cleavage at this site led to activation of the GDF11. Without cleavage at the BMP1 site, the precursor was resistant to cleavage by furin.

### Example 3: Proteolytic Activation of GDF11 Generates a Soluble Complex

Visual examination of the AspN/23 hour furin sample revealed that a precipitate had formed and settled from the digest. SDS-PAGE analysis of the supernatant and precipitate fractions under reducing and non-reducing conditions (FIG. 2C lanes 8, 9) revealed that all of the mature GDF11 was in the soluble fraction. The precipitate contained the major prodomain fragment corresponding to residues 122-298. An unexpected finding from the study was that the proteolytically activated GDF11 was soluble at neutral pH. Poor solubility under non acidic conditions is a common characteristic of most members of the TGF-β family and commercial preparations of the proteins routinely utilize formulations that have a pH less than 5 and at concentrations of 10-100 μg/ml in the presence of carrier protein to prevent aggregation/precipitation. In fact the mature GDF11 standard that was purchased was only soluble under acidic conditions and precipitated when the pH was raised to neutral. To better understand this property of AspN/furin cleavage product, the preparation was subjected to SEC and the SEC-purified sample was analyzed by mass spectrometry. FIG. 3 shows an SEC profile of the material and analysis of the column fractions by SDS-PAGE. The SEC profile showed a broad elution peak with an apparent molecular weight of >50 kDa. No high molecular weight aggregates were detected in the preparation. The >50 kDa size was significantly larger than the expected mass of 25 kDa. In column fractions that contain the peak of the GDF11 (lanes 3-5), mature GDF11 (25 kDa non-reducing, 14 kDa reducing) as well as the broad band with molecular weight of 6-16 kDa under reducing and non-reducing conditions were detected. When deglycosylated and analyzed by mass spectrometry, the 6-16 kDa band was identified as a fragment of the prodomain containing amino acids 60-114 and residues 60-112 resulting from secondary cleavage of the same peptide at amino acid 112. (FIGS. 5B and C). The identity of the peptides 60-112 and 60-114 were confirmed by collision-induced dissociation (CID) tandem MS/MS.

When the 60-114 peptide sequence from GDF11 was overlaid onto the published crystal structure of latent TGF-β (Shi et al., (supra)), it mapped to a feature coined as the lasso/straight jacket region that wraps around the fingers of the mature domain. The GDF11 peptide contains the entire α1 helix, lasso, and a portion of α2 helix that due to truncation may, or may not assume the helical conformation seen in the crystal structure. In the latent TGF-β structure, the α1 helix is buried in the interface between the growth factor monomers, the lasso forms an extended loop that forms hydrophobic contacts with the tips of the mature domain fingers, and the α2 helix occupies an interface on the surface of finger 2 that overlaps with the type II receptor interface of the BMP members of the TGF-β superfamily. For example, this interface can be observed in the structure of BMP2 in complex with bone morphogenetic protein receptor type Ia (BMPRIa) and activin receptor type IIA (ActRIIA), which is deposited under PDB-ID 2GOO.pdb (Allendorph et al., Proc. Natl. Acad. Sci. USA, 103:7643-7648 (2006)). Sequence alignment of TGF-β1, BMP2, and GDF11 and structure inspection of the residues that point out of the convex interface of the finger 2 region reveal that these regions are similarly characterized by a combination of hydrophobic and polar residues. Both BMP2 and GDF11 bind ActRIIA in this region (Yadin et al., Cytokine Growth Factor Rev., 27:13-34 (2016)), while TGF-β1 does not. However, due to the similar characteristics of the type II receptor binding sites of BMP2 and GDF11 and the structurally equivalent region on the surface of TGF-β1, this region of GDF11 is likely to bind to the prodomain α2 helix of GDF11. Furthermore, the α2 helix is comparatively unchanged between the open conformation of BMP9 (4YCG.pdb, 4YCI.pdb, Mi et al., Proc. Natl. Acad. Sci. U.S.A., 112:3710-37152015, FIG. 3B) and the closed conformation of the TGF-β1 prodomain (3RJR.pdb, Shi et al., (supra) 2011) when examined relative to the mature domain's finger 2: in both cases, it occupies the BMP group's type II receptor binding site. By superposing and comparing structures for mature/active TGFβ1 (4KV5.pdb) and for latent TGFβ1 (3RJR.pdb), we observed that in the latent complex, the prodomain α1 helix displaces the active growth factor wrist helix from its position, thus structurally distorting the wrist epitope that is characteristic for the TGFβ1-3 and BMP/GDF groups of the TGF-β superfamily. Since the GDF11 complex that was produced is in an active state, the latent TGF-β structure is not a good model for the interactions of the GDF11 propeptide with mature GDF11.

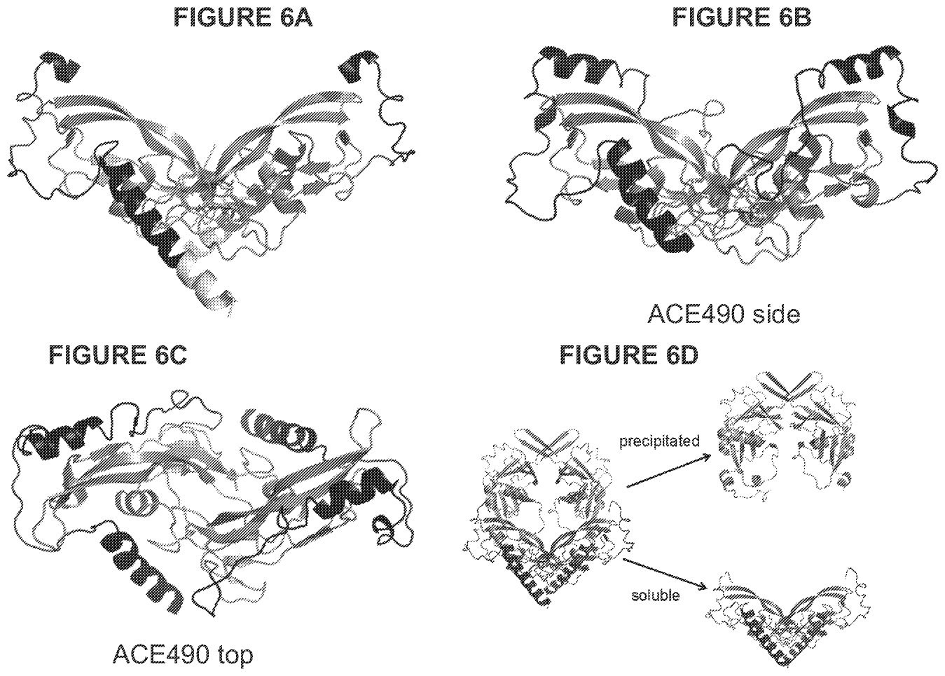

FIG. 6A shows a model of the mature GDF11/propeptide residue 60-114 complex that takes into account potential structural differences with the latent TGF-β1 structure and incorporates the assumption that the GDF11 α2 helix occupies the type II receptor binding site. The concave type I receptor binding site provides sufficient space for two helices, as evidenced by the BMP9 prodomain α5 helix, which runs parallel to the wrist helix. By contrast, the GDF11 α1 helix would have to run antiparallel because it starts at the finger 1 side of the molecule. In none of the docking models that were explored did the α1 helix settle into the expected parallel position. As a control, when docking the GDF11 α1 helix against the GDF11 monomer using PIPER, it clustered in a position similar to its position relative to the monomer in the TGFβ1 latent structure for most of the highly populated clusters. As a second control, the follistatin ND helix (3HH2.pdb, Cash et al., EMBO J., 28:2662-2676 (2009)) was docked into the mature GDF11 biological unit. It readily populates the largest clusters in the expected conformation antiparallel to the GDF11 wrist helix. This indicated that if the GDF11 α1 helix had evolved to occupy a position parallel to the wrist helix, chances are that it would have been observed in these docking models.

GDF11 peptide 60-112/114 is twice as long as the 24-residue minimum inhibitory peptide located within the putative α1 helix of myostatin (Shi et al., 2011) that binds with 30 nM affinity and blocks its function (Takayama et al., J. Med. Chem., 58:1544-1549 (2015)). In contrast with myostatin and its inhibitory peptide, association of GDF11 peptide 60-112/114 with mature GDF11 did not impact its activity and is indistinguishable from the activity of the mature GDF11 alone (FIG. 4C). As a positive control for the study, Fc fusion proteins of the prodomain designed essentially as described previously (Ge et al., Mol. Cell. Biol., 25:5846-5858 (2005)) were produced and tested for their ability to inhibit GDF11-induced signaling in the luciferase reporter assay (FIG. 4D). Both prodomain fusion proteins were potent inhibitors of GDF11 activity.

The binding characteristics of the GDF11 peptide 60-112/114 for mature GDF11 residues 299-407 and for AspN fragment residues 122-407 were assessed using an Octet Red system. For these studies, full length GDF11 was biotinylated through the single glycan in full length GDF11 (Asn-94), which fortuitously is present in the 60-112/114 peptide (see FIG. 1). Preparations of the biotinylated GDF11 that had been treated with AspN alone, treated with AspN and furin, or of the purified biotinylated GDF11 peptide 60-112/114 (FIG. 2C, lane 11) were captured on Streptavidin biosensors and evaluated for dissociation over time (FIG. 3B). The amplitude of the response on the sensor was proportional to the size of the complex, yielding signals of 0.5 nm for the peptide itself (6-11 kDa), 1.2 nm for the complex of the peptide with the mature domain (40 kDa), and 3.5 nm for the Asn-N only digested samples (100 kDa). The dissociation kinetics of the samples were very slow, as 10% or less of the signal was lost following 60 min incubation in dissociation buffer. The dissociation rates for the AspN-treated alone and AspN/furin-treated samples were indistinguishable indicating high-affinity binding of the peptide to both forms of the protein. The binding characteristics of GDF11 samples for TGF-β Type I and II receptors were also assessed using an Octet readout (FIG. 3C). Following loading of biotinylated GDF11 that had been treated with AspN alone or AspN/furin, the Streptavidin Octet tips were treated with recombinant human Activin RIB-Fc and Activin RIIA-Fc proteins. Only binding of the Type II receptor to the AspN/furin GDF11 sample was observed and neither receptor bound to AspN-treated GDF11. As confirmation of this finding, the AspN/furin preparation was Alexa488-labeled, mixed with Activin RIIA-Fc, subjected to SEC, column fractions were collected, and the Activin RIIA-Fc/GDF11 complex was analyzed by SDS-PAGE monitoring the fluorescence of the GDF11 fragments. Association of the AspN/furin preparation with the receptor resulted in the formation of a complex that migrated on the SEC column with a molecular weight of >100 kDa with baseline separation from the free AspN/furin GDF11 fragment alone. Fractions corresponding to the complex contained both fluorescently labeled mature GDF11 and labeled 60-112/114 peptide (data not shown). The presence of the peptide in the complex indicates that binding to the receptor does not displace the N-terminal prodomain peptide.

### Example 4: Processing of GDF11 with Plasmin and Trypsin

To assess whether the differences in the susceptibility of full length and AspN-treated GDF11 to processing were unique to furin or reflected an inherent property of the GDF11 that could be recapitulated with other proteases, the susceptibility of GDF11 to limited digestion with plasmin and trypsin was evaluated. Plasmin and trypsin cleave after arginine and lysine residues and thus should cleave at the furin site, although they would be expected to be less selective than furin. In fact whereas GDF11 contains a single furin site, there are 39 lysines and arginines that could be targets for plasmin or trypsin. Previously, both enzymes were successfully used for studies with other TGF-β family members; plasmin for proteolytic activation of AMH (Di Clemente et al., Mol. Endocrinol., 24:2193-2206 (2010)) and trypsin for processing artemin, a member of the GFL subfamily (Silvian et al., Biochemistry, 45:6801-6812 (2006)). Similar to treatment effects with furin, full length GDF11 was more resistant than AspN-treated GDF11 to digestion with plasmin or trypsin (FIG. 2C, plasmin lanes 1 and 5, trypsin lanes 2 and 6). Under conditions that led to extensive cleavage of AspN-treated GDF11, full length GDF11 was partially cleaved by plasmin producing fragments with molecular weights of 50, 35, 25, and 23 kDa. These bands were present in the furin only treated GDF11 sample (lane 3), but at much lower concentrations. In contrast, without prior AspN treatment, the full length GDF11 was resistant to cleavage with trypsin (lane 2). Together these studies revealed that furin, plasmin, and trypsin are all sensitive to structural changes in GDF11 resulting from AspN treatment.

The cleavage products produced with AspN and plasmin under reducing and non-reducing conditions (FIG. 2C, lane 5) looked very similar to the pattern seen with furin (lane 7) both for the mature GDF11 (non-reduced 25 kDa, reduced 14 kDa) and prodomain fragments (N-terminal 6-16 kDa glycopeptide, C-terminal 23/22 kDa fragment). The production of mature GDF11 residues 299-407 following plasmin treatment was confirmed by MS (predicted mass 12457.4 Da, observed mass 12458 Da). Consistent with the lower specificity of the enzyme, plasmin treatment also led to cleavage at a second site within the dibasic KRSRR (SEQ ID NO:18) recognition sequence to yield a 3 amino acid longer fragment containing residues 296-407 (predicted mass 12856.8 Da, observed mass 12856 Da). With a longer incubation time with plasmin, cleavage at Lys-352 occurred, although it was less reactive than the other sites. Trypsin treatment of the GDF11 AspN fragment was less selective (lane 6) than furin or plasmin and lead to cleavage at several additional sites, notably the 30 kDa band and distinct low molecular weight bands that overlapped with the 6-16 kDa glycopeptide.

### Example 5: Designing Propeptide/Mature GDF11 Fusion Proteins

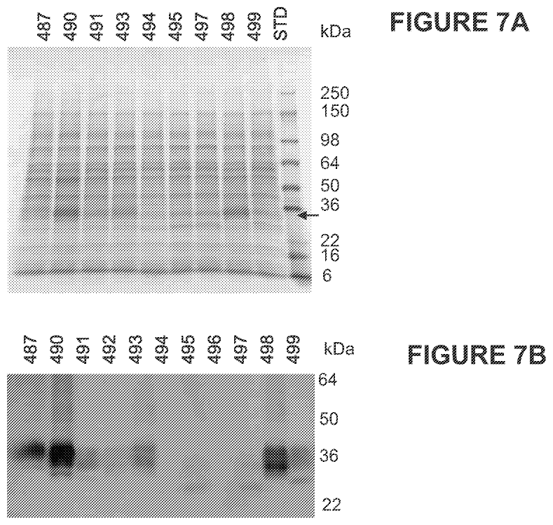

No GDF11 was detected when the mature domain was expressed in CHO cells alone in absence of the prodomain. To test if a genetic fusion of the 60-114 peptide with the mature domain of GDF11 could improve the expression of GDF11, we used the latent TGF-β structure and the hypothetical structure-based sequence alignment of GDF11 to TGF-β (Shi et al., (supra) 2011) to model potential G₄S (SEQ ID NO:4) linker lengths in a series of 11 different constructs and these constructs were expressed in CHO cells (Table 1). Because of the close proximity of the N and C termini from both chains of mature GDF11 in the dimer, constructs were designed in two orientations, with the prodomain peptide attached at the N and C terminus of the mature domain. G₄S (SEQ ID NO:4) spacers of varying lengths were incorporated into the designs between the peptide and mature domain to allow proper assembly of the domains. Some of the constructs contained deletions of up to 11 amino acids from the N-terminus of the peptide that, from the model, did not make contacts with the mature domain. Other constructs contained additional amino acids at the C-terminus that completed the α2 helix (FIG. 6). An 8-His (SEQ ID NO:3) tag was engineered at the N-terminus of all the constructs to facilitate their purification. FIGS. 6B and 6C show different orientations of the model for ACE490, which proved to be one of the more successful designs (discussed below). From the model, the 3XG4S (SEQ ID NO:19) linker allows for proper contacts between the prodomain and mature domain to form and was predicted to be the minimal size that would allow the domains to assemble. FIGS. 7A and B show Coomassie stained and western SDS-PAGE profiles from conditioned medium of CHO cells expressing the 11 constructs. Expression of the peptide fusions led to a wide range of measureable titers (Table 2) with levels for two of the constructs exceeding 50 mg/L. Surprisingly, high titers were only observed when the full α2 helix (containing D122) was incorporated (ACE490 and ACE498). In fact, 20-fold lower titers occurred when truncated versions ending in L114 were used. High titers were only observed when the propeptide was attached at the N-terminus of the mature GDF11 domain. Poor expression occurred when the same prodomain sequences were attached at the C-terminus of the mature domain. The expression levels achieved with the ACE490 and ACE498 designs were greater than that observed when the full length GDF11 protein was expressed.

TABLE 2

| Construct | Protein sequence attributes | Titer | SEQ ID NO: |
|---|---|---|---|
| pACE487 | 8xHis-TEV-GDF (G61-D122)-G-4xG4S-GDF (N299-S407) | ++ | 5 |
| pACE490 | 8xHis-TEV-GDF (S71-A123)-3xG4S-GDF (N299-S407) | ++++ | 6 |
| pACE491 | 8xHis-TEV-GDF (G61-L114)-3xG4S-GDF (N299-S407) | + | 7 |
| pACE492 | 8xHis-TEV-GDF (D60-L114)-3xG4S-GDF (N299-S407) | +/− | 8 |
| pACE493 | 8xHis-TEV-GDF (D60-L114)-4xG4S-GDF (N299-S407) | + | 9 |
| pACE494 | 8xHis-TEV-GDF (N299-S407)-2xG4S-GDF (S71-L114) | − | 10 |
| pACE495 | 8xHis-TEV-GDF (N299-S407)-3xG4S-GDF (S71-L114) | − | 11 |
| pACE496 | 8xHis-TEV-GDF (N299-S407)-3xG4S-GDF (D60-L114) | − | 12 |
| pACE497 | 8xHis-TEV-GDF (N299-S407)-3xG4S-SP-GDF (R72-L114) | − | 13 |
| pACE498 | 8xHis-TEV-GDF (D60-D122)-2xG4S-GDF (N299-S407) | ++++ | 14 |
| pACE499 | 8xHis-TEV-GDF (N299-S407)-3xG4S-GDF (S71-D122) | + | 15 |
| pACE382 | GDF (N299-S407) | − | 20 |

A series of 11 prodomain-mature fusion constructs were engineered and expressed in CHO cells. All contained the GDF11 signal peptide linked to an 8 histidine (SEQ ID NO: 3) affinity tag with a TEV protease cleavage N-terminal to the designs. G4S (SEQ ID NO: 4) spacer sequences of varying lengths were included in the designs. Pro sequences are underlined and mature domain sequences are boldened. Relative titers for each construct were assessed directly from the conditioned medium by Western blotting, using the anti-GDF11 C-terminal peptide polyclonal antibody for detection (see FIG. 7B). As control, a mature domain only construct ACE382 was expressed using the GDF11 signal sequence but no His tag.

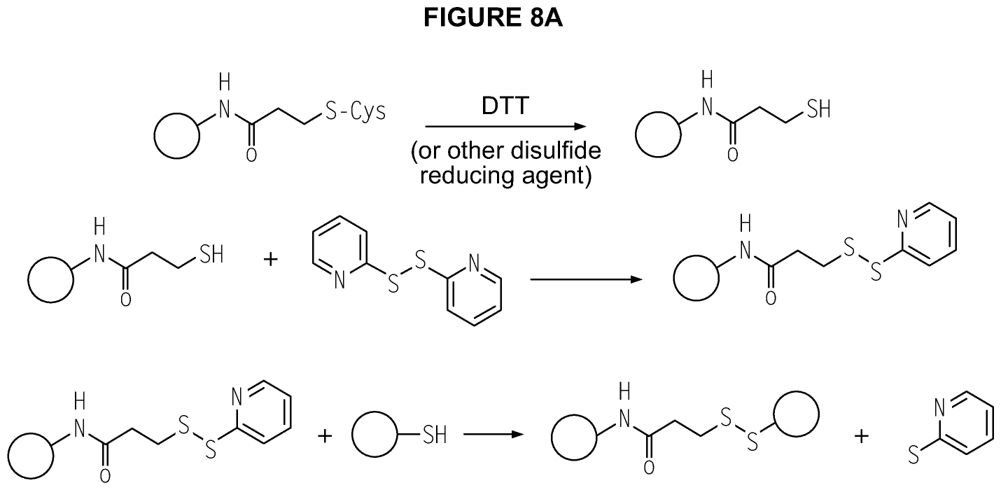

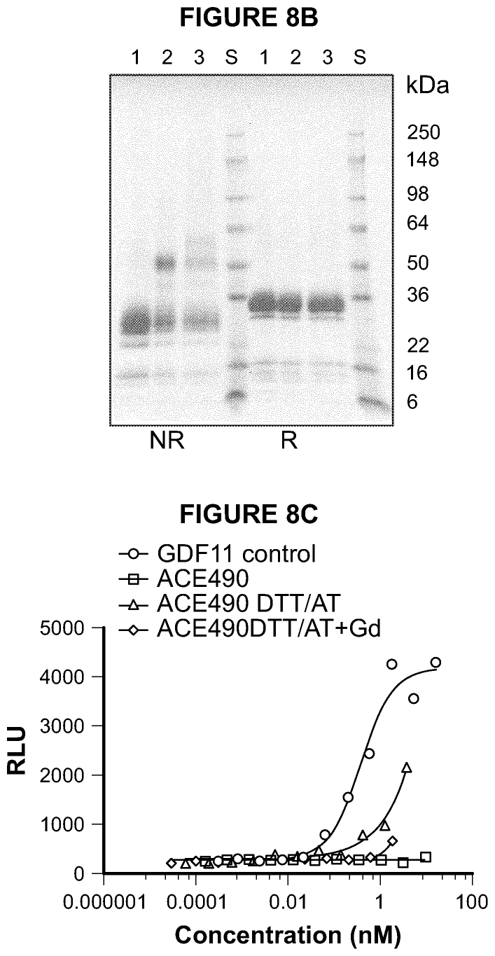

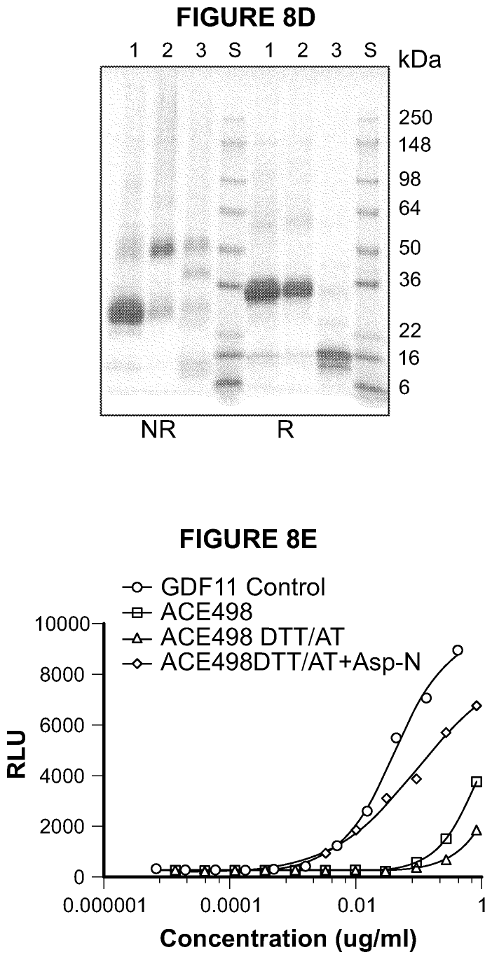

ACE490 and ACE498 were purified from the condition medium by column chromatography on Ni Excel Sepharose. Non-reducing SDS-PAGE analyses of the purified preparations (FIGS. 8B and D) of purified preparations of ACE490 and ACE498 revealed that neither sample had formed the interchain disulfide that covalently links the mature GDF11 dimer when it is assembled properly. Further assessment by SEC showed that both products eluted from the sizing column as monomers (data not shown). When these samples were tested for function in the reporter assay, they were both inactive (see FIGS. 8C and 8E). Next it was determined if dimer formation could be promoted by redox. For these studies, the proteins were reduced with DTT, then half of the preparation was treated with aldrithiol to activate the cysteine. This was then added to the other half of the protein to drive dimerization. FIG. 8A shows a schematic summarizing the redox steps. This treatment of ACE490 led to significant dimer formation. FIG. 8B shows an SDS-PAGE analysis of the redox product. No dimer formation occurred without the aldrithiol activation step. When this preparation was tested in the SMAD 2/3 reporter assay, the sample recovered about 10% of the activity observed for mature GDF11 (FIG. 8C). Aldrithiol/redox treatment of ACE498 also led to dimer formation, but with the shorter G₄S (SEQ ID NO:4) linker, the preparation was inactive in the SMAD 2/3 reporter assay (FIGS. 8D and 8E). Interestingly, when this preparation was treated with endoproteinase AspN, the specific activity in the reporter assay was increased to about 50% of the GDF11 standard, indicating that dimerization followed by cleavage at the pro-mature domain interface was required for activation. We were unable to promote cleavage of ACE490 redox product with AspN protease. From the characteristics of the ACE490 and ACE498 constructs and activation following simple redox without the need for a refolding step, it can be inferred that the cysteine knot motif properly formed during expression, but the absence of the Asp-122-Arg-229 region of the prodomain or improper alignment of the Asp-60-Asp-120 sequences in its complex with the mature domain prevented dimerization.

### Example 6: Formulating Mature GDF11 with a Synthetic Propeptide to Improve Solubility

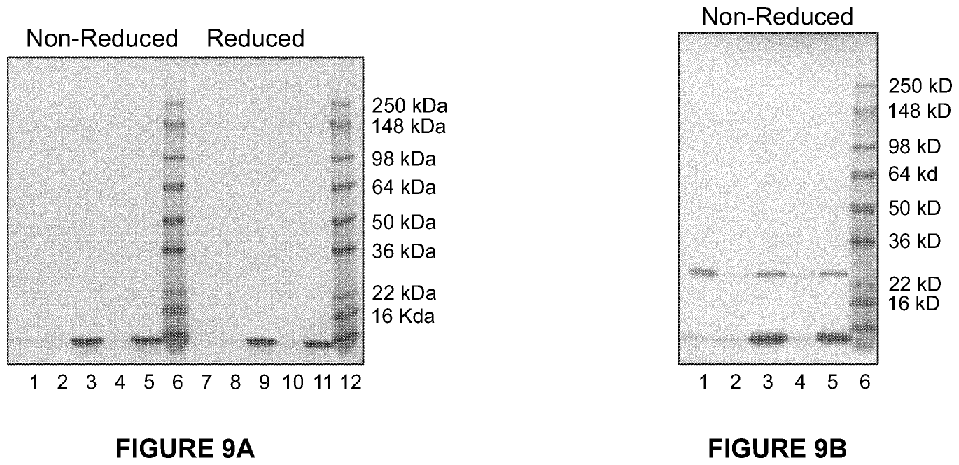

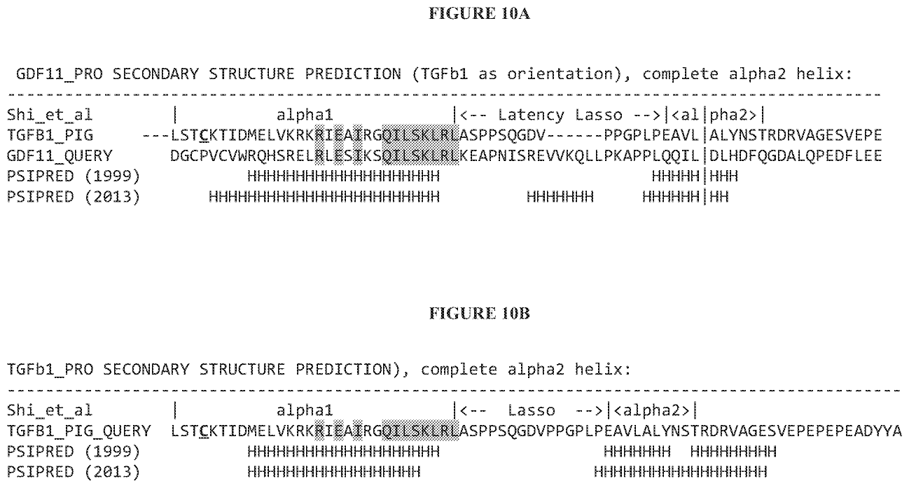

GDF11 propeptide NH₂-SPRELRLESIKSQILSKLRLKEAPNISREVVKQLLPKAPPLQQIL (SEQ ID NO:21)-COOH was custom synthesized by New England Peptide. A 10 mg/mL stock solution of the peptide was prepared in 20 mM sodium phosphate pH 7.0, 150 mM NaCl. Carrier free mature GDF11 (R&D systems) was reconstituted at 400 μg/mL in 4 mM HCl. 2 (5 μL) of the mature GDF11 was diluted with 5 μL buffer (100 mM Tris HCl pH 7.5, 100 mM Tris pH 8.5 or 100 mM sodium phosphate pH 6.5) in the presence or absence of 10 μg of the propeptide. The samples were incubated at room temperature for 1 hour, then centrifuged at 10,000 rpm for 10 minutes in an Eppendorf centrifuge. The supernatants were analyzed by SDS-PAGE on a 4-20% gradient gel. The gel was stained with Simply blue. At all three pHs tested, mature GDF11 was only soluble in the peptide containing formulations (FIGS. 9A and 9B). In the absence of the propeptide, there was a large loss of GDF11 in the supernatant fractions, as evident by reduction in staining compared to the control lane (which shows the input amount of GDF11 tested).

### Example 7: Peptides Shorter than the Recovered Endoproteinase Asp-N Digest (55 Residues) of the GDF11 Prodomain Designed to Bind to the Cleaved Mature Domain

Synthetic peptides were designed to better understand how the propeptides from the AspN digest bind to the GDF11 mature domain.

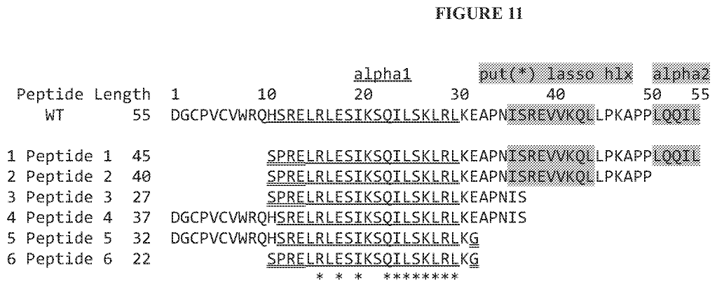

For peptide synthesis to be feasible, candidate peptides should be shorter than the 55 residues observed in the AspN digest, up to a range of 30-45 residues. Based on the GDF11 prodomain peptide 60-114, six peptide variants of different lengths were designed (FIG. 11), which included or excluded the latency lasso and the helix or helices described below. All variants contain a 19-residue peptide 71-87 segment of the α1 helix that is most likely helical, (SRELRLESIKSQILSKLRL (SEQ ID NO:22), underlined in the constructs in FIG. 11), because it is 58% identical with the equivalent portion of the α1 helix of porcine TGF-β1 (Shi et al., Nature, 474:343-9 (2011)), (FIG. 13), and because it is predicted to be helical by secondary structure prediction methods discussed below (FIG. 10).

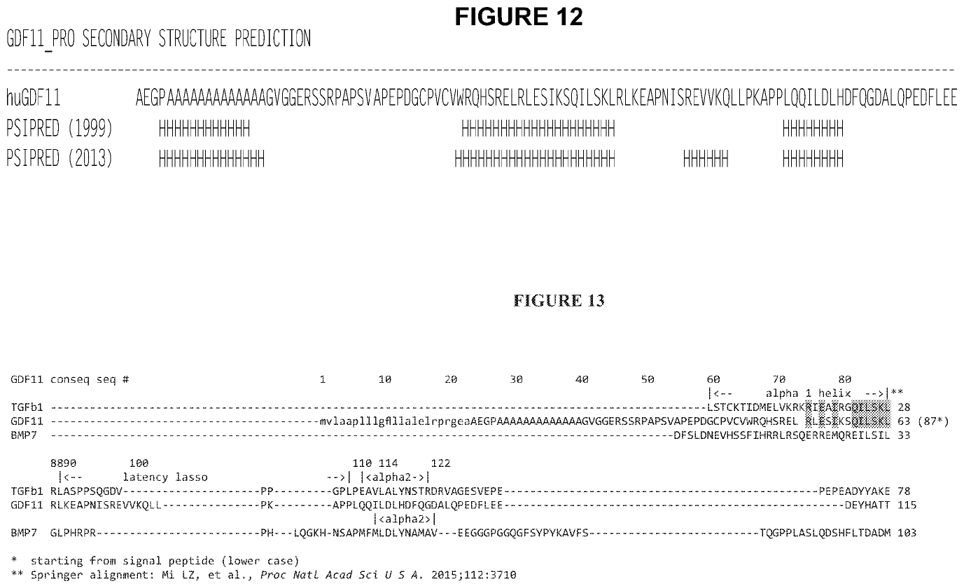

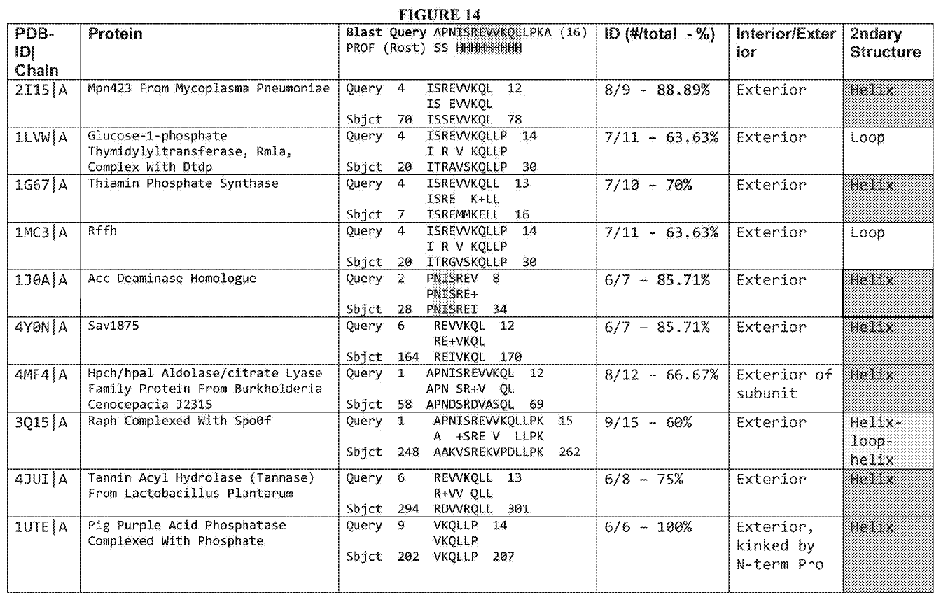

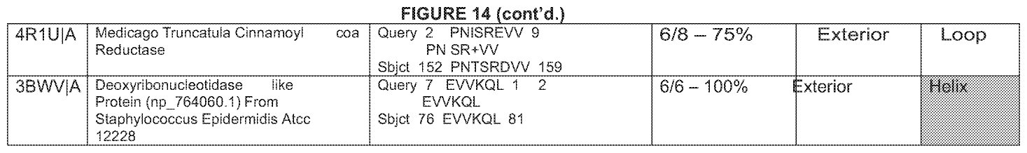

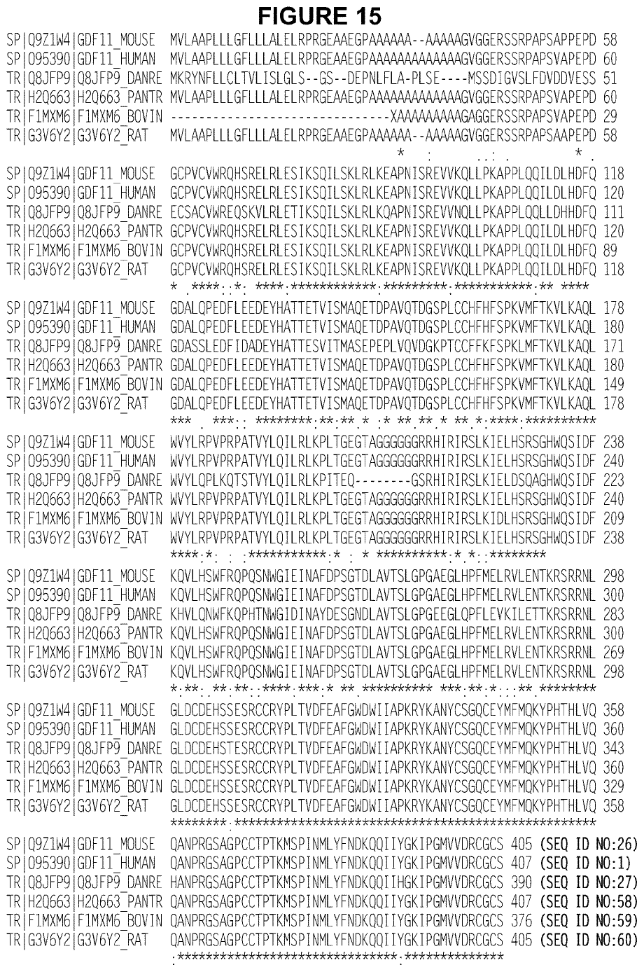

Human GDF11 very likely has more than two helices before the AspN cleavage site. There are ambiguous results about the GDF11 lasso region following the α1 helix: specifically, whether or not there is another α-helix between the α1 and α2 helices. Secondary structure of the prodomain sequences of both human GDF11, residues 60-132, and the equivalent porcine TGF-β1 sequence was predicted using PSIPRED method (Jones et al., Journal of Molecular Biology, 292, 195-202 (1999)), and using the updated PSIPRED method (PSIPRED: Buchan et al., Nucleic Acids Research, 41 (W1):W340-W348 (2013)). Results are shown in FIG. 10. The query sequence was deliberately extended beyond the AspN cleavage site, allowing the servers to predict the entire length of the α2 helix. In addition to confirming helical content for the α1 and α2 helices by both methods, PSIPRED (2013) predicts a third helix located between these two helices. Furthermore, when submitting the complete first 132 residues of the human GDF11 prodomain to the same prediction methods (FIG. 12), both methods predict another α-helix for the 13-Ala putative transmembrane helix segment 29-41 (FIG. 13) of the GDF11 prodomain and the additional helix between the α1 and α2 helices, thus suggesting a total of four α-helices in the prodomain before the AspN cleavage site. Similar trends were observed when more secondary structure prediction methods were consulted (data not shown): seven out of nine methods predict the 13-Ala stretch to be helical, amounting to a total of three helices, and four out of nine predict four helices, including the 13-Ala stretch and the additional helix between the α1 and α2 helices. Lastly, Blast (Altschul et al., Journal of Molecular Biology, 215, 403-410 (1990)) was used to query the sequence of human GDF11 prodomain peptide 92-107 (sequence APNISREVVKQLLPKA (SEQ ID NO:23)), which contains the putative α-helix between the α1 and α2 helices. With this Blast query, sequences of proteins in the Protein Data Bank (PDB; Berman et al., Nucleic Acids Research, 28, 235-242 (2000)) were searched. Results show that eight out of the first twelve non-redundant homologous hits to that query sequence have helical secondary structure (FIG. 14).

Peptides were designed that included the presumed al- and/or α2-helices of the porcine TGF-β1 pro-domain/mature domain complex structure (3RJR.pdb, Shi et al., Nature, 474:343-349 (2011)), and the putative helix in between the former two. Based on a multiple sequence alignment of 33 TGF-β family members, which include GDF11 and myostatin (GDF8), it was suggested that helices homologous to α1 and α2 are present throughout the family. In porcine TGF-β1, these helices are connected through an unstructured loop, termed the “latency lasso”, whose contacts with the mature domain's fingers 1 and 2 likely contribute to binding. The lasso region is rich in proline (6 out of 15 residues, or 40%), which interact with various tryptophans of the mature domain. The latency lasso of GDF11 is six residues longer than the equivalent region of porcine TGF-β1, indicating structural differences. As discussed above, secondary structure prediction methods and a Blast search predict helical content for the latency lasso-equivalent region of human GDF11 while this is not the case for the latency lasso region of TGF-β1. GDF11's lasso region, while still proline-rich (4 out of 21, or 19%, vs. a 5% average value for vertebrate proteins), has fewer prolines than porcine TGF-β1. These prolines may function as helix breakers or starters, or for interactions with the mature domain as in TGF-β1. The potential occurrence of this additional “lasso helix” was taken into account in the peptide designs.

In four of the peptide designs, the length of the α1 helix was shortened for several reasons. First, there is no sequence identity between the first half of the GDF11 α1 helix (residues 60-70, sequence DGCPVCVWRQH (SEQ ID NO:24)) and the equivalent sequence of porcine TGF-β1. Second, this section contains five residues that rank at the bottom of helix propensity scales (D,G,C) (Pace et al., Biophysical J, 75, 422-427 (1998), Table 4) or are known as a “helix breaker” (P). Lastly, only two out of nine secondary structure prediction methods predict helical content for most of this segment.

We aimed to ensure helix formation through N-terminal and C-terminal helix caps. N-terminal caps are reported to stabilize monomeric helix formation by up to 2 kcal/mol, while there is no discernable energy advantage for C-caps (Pace et al., Biophysical J, 75, 422-427 (1998)). Helix capping can follow one of seven commonly observed short-range conformational patterns formed by the local sequence preceding or following the α-helix (three N-terminal and four C-terminal helix caps), or the helix can be capped by long-range intra-molecular or intermolecular interactions, see Aurora and Rose (Aurora and Rose, Protein Science, 7, 21-38 (1998)).

Alternatively, the first N-terminal pattern classified by Aurora and Rose (Aurora and Rose, (supra)) has been called the “SXXE box”, or “the hydrophobic staple” (Pace et al., (supra)), in which the polar S is the N-cap, without a preceding hydrophobic residue. In the peptide designs the latter pattern was applied. In addition, a C-capping glycine (termed the “Schellman cap”), was also employed.

### Description of Peptides (see FIG. 11):

Four peptides have a shortened α1 helix to prune potentially non-helical residues from the helical segment. Four other peptides extend beyond the α1 helix in case additional helices are essential for binding, or in case some apparently disordered regions are needed for binding or because they form natural helix capping motives. The peptides are set forth in FIG. 11.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-6. (canceled)

7. An isolated protein comprising, in order, a first amino acid sequence and a second amino acid sequence linked directly via a peptide linker of 5 to 100 amino acids in length, wherein the first amino acid sequence is 52 to 65 amino acids in length and comprises amino acids 60 to 114 or 71-123 of SEQ ID NO:1, and the second amino acid sequence comprises an amino acid sequence that is at least 90% identical to amino acids 299-407 of SEQ ID NO:1, wherein the protein when activated by dimerization and/or by dimerization and proteolytic cleavage induces SMAD 2/3 phosphorylation in a Kinase Induced Receptor Activation Assay.

8.-33. (canceled)

34. A nucleic acid sequence encoding the protein of claim 7.

35. An expression vector comprising the nucleic acid sequence of claim 34.

36. A host cell comprising the expression vector of claim 35.

37. A method of producing a protein, comprising culturing the host cell of claim 36 in a culture medium under conditions in which the protein of claim 7 is produced by the host cell and secreted into the culture medium.

38. A method of making an activated GDF11 protein, the method comprising:
(a) providing a GDF11 protein;
(b) subjecting the protein to a disulfide reducing agent to create a first composition;
(c) dividing the first composition into a second and a third composition;
(d) subjecting the second composition to a cysteine activating agent to create a fourth composition;
(e) combining the fourth composition with the third composition to create a fifth composition; and
(f) treating the fifth composition with a protease that cleaves at the BMP1 site of the protein, thereby making an optimally activated GDF11 protein.

39. The method of claim 38, wherein the disulfide reducing agent is DTT.

40. The method of claim 38, wherein the cysteine activating agent is aldrithiol.

41. The method of claim 38, wherein the protease that cleaves at the BMP1 site of the protein is endoproteinase AspN.

42. The method of claim 38, further comprising formulating the fifth composition at a pH of 5.0 to 6.5.

43. The method of claim 42, wherein the pH is 5.5.

44. The method of claim 38, wherein the protein is produced by a mammalian cell.

45. The method of claim 44, wherein the mammalian cell is a CHO cell.

46. A method of producing an activated human GDF11 protein, the method comprising:
contacting a GDF11 protein with a first protease that cleaves at a BMP1 site of the GDF11 protein; and
contacting the protein with a second protease that is furin, plasmin, or trypsin.

47. The method of claim 46, wherein the GDF11 protein is a full length human GDF11 protein.

48. The method of claim 47, wherein the full length human GDF11 protein is obtained from a mammalian cell.

49. The method of claim 48, wherein the mammalian cell is a CHO cell.

50. The method of claim 46, wherein the protease that cleaves at the BMP1 site of the GDF11 protein is endoproteinase AspN.

51. The method of claim 46, wherein the second protease is furin.

52. The method of claim 46, further comprising formulating the GDF11 protein as a pharmaceutical composition.

53. The method of claim 52, wherein the pharmaceutical composition has a pH in the range of 5.0 to 6.5.

54. The method of claim 53, wherein the pharmaceutical composition has a pH of about 5.5.

55. A method of preparing a protein formulation, the method comprising combining a first polypeptide that comprises an amino acid sequence that is at least 90% identical to:
(i)
(SEQ ID NO: 21)
SPRELRLESIKSQILSKLRLKEAPNISREVVKQLLPKAPPLQQIL;
(ii)
(SEQ ID NO: 28)
SPRELRLESIKSQILSKLRLKEAPNISREVVKQLLPKAPP;
(iii)
(SEQ ID NO: 29)
SPRELRLESIKSQILSKLRLKEAPNIS;
(iv)
(SEQ ID NO: 30)
DGCPVCVWRQHSRELRLESIKSQILSKLRLKEAPNIS;
(v)
(SEQ ID NO: 31)
DGCPVCVWRQHSRELRLESIKSQILSKLRLKG;
or
(vi)
(SEQ ID NO: 32)
SPRELRLESIKSQILSKLRLKG,
with
a second polypeptide that comprises an amino acid sequence that is at least 90% identical to amino acids 299-407 of human GDF11 (SEQ ID NO:1).

56. The method of claim 55, wherein the second polypeptide consists of amino acids 299-407 of SEQ ID NO:1.

57. The method of claim 55, wherein the first polypeptide consists of the amino acid sequence of SEQ ID NO:21.

58. The method of claim 55, further comprising adjusting the pH of the formulation to between 5.0 and 6.5.

59. The method of claim 58, wherein the pH of the formulation is 5.5.

60.-61. (canceled)


SEQUENCE LISTING (89 sequences)

SEQ ID NO: 1 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 2 - DNA, 1224 bp, Homo sapiens
atggttcttgcagctcctctgctgctgggcttcctgctcctcgctctggagctgcgacccagaggtgaggcggccgagggacctgcagctgcagctgctgctgcagctgccgcagcagctgcaggggtcggaggagagcgctccagccggccagctccttctgtggctcctgagccagacggctgccccgtgtgcgtttggcggcagcacagccgcgagctgcgcctagagagcatcaagtcgcagatcctgagcaaactgcggctcaaggaggcgcccaacatcagccgcgaggtggtgaagcagttgctgcccaaggcgccgccgctgcaacagatcctggacctacacgacttccagggcgacgcgctgcaacccgaggacttcctggaggaggacgagtaccacgccaccaccgagaccgtcattagcatggcccaggagacggacccagcagtacagacagatggcagccctctctgctgccatttccacttctcacccaaggtgatgttcacaaaggtcctgaaggcccagttgtgggtgtacctacggcctgtaccccgcccagccacagtctacctgcaaatcctgcgactaaaacccctaactggggaagggaccgcaggtggaggtggtggaggccggcgtcacatccgtatccgctcactgaagattgagctgcactcacgctcaggccattggcagagcatcgacttcaagcaagtgctacactcttggttccgccagccacagagcaactggggcatcgagatcaacgcctttgatcccagtggtacagacctggctgttacatctctggggccgggagccgaggggctgcatccattcatggagcttcgagtcctagagaacacaaaacgttcccggcggaacctgggtctggactgcgacgagcactcaagcgagtcccgctgctgtaggtatcctctcacagtggactttgaggctttcggctgggactggatcatcgcacctaagcgctacaaggccaactactgctccggccagtgcgagtacatgttcatgcagaagtacccgcatacccatttggtgcagcaggccaatccaagaggctctgctggaccctgttgtacccctaccaagatgtccccaatcaacatgctctacttcaacgacaagcagcagatcatctacggcaagatccctggcatggtggtggatcgctgtggctgctcttga

SEQ ID NO: 3 - protein, 8 aa, Artificial Sequence
  source: /note=“Description of Artificial Sequence: Synthetic peptide”
HHHHHHHH

SEQ ID NO: 4 - protein, 5 aa, Artificial Sequence
  source: /note=“Description of Artificial Sequence: Synthetic peptide”
  source: /note=“See specification as filed for detailed description of substitutions and preferred embodiments”
GGGGS

SEQ ID NO: 5 - protein, 227 aa, Artificial Sequence
  source: /note=“Description of Artificial Sequence: Synthetic polypeptide”
METDTLLLWVLLLWVPGAHAHHHHHHHHENLYFQSGCPVCVWRQHSRELRLESIKSQILSKLRLKEAPNISREVVKQLLPKAPPLQQILDLHDFQGDGGGGGSGGGGSGGGGSGGGGSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 6 - protein, 211 aa, Artificial Sequence
  source: /note=“Description of Artificial Sequence: Synthetic polypeptide”
METDTLLLWVLLLWVPGAHAHHHHHHHHENLYFQSRELRLESIKSQILSKLRLKEAPNISREVVKQLLPKAPPLQQILDLHDFQGDAGGGGSGGGGSGGGGSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 7 - protein, 212 aa, Artificial Sequence
  source: /note=“Description of Artificial Sequence: Synthetic polypeptide”
METDTLLLWVLLLWVPGAHAHHHHHHHHENLYFQGCPVCVWRQHSRELRLESIKSQILSKLRLKEAPNISREVVKQLLPKAPPLQQILGGGGSGGGGSGGGGSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 8 - protein, 214 aa, Artificial Sequence
  source: /note=“Description of Artificial Sequence: Synthetic polypeptide”
METDTLLLWVLLLWVPGAHAHHHHHHHHENLYFQSDGCPVCVWRQHSRELRLESIKSQILSKLRLKEAPNISREVVKQLLPKAPPLQQILGGGGSGGGGSGGGGSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 9 - protein, 219 aa, Artificial Sequence
  source: /note=“Description of Artificial Sequence: Synthetic polypeptide”
METDTLLLWVLLLWVPGAHAHHHHHHHHENLYFQSDGCPVCVWRQHSRELRLESIKSQILSKLRLKEAPNISREVVKQLLPKAPPLQQILGGGGSGGGGSGGGGSGGGGSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 10 - protein, 197 aa, Artificial Sequence
  source: /note=“Description of Artificial Sequence: Synthetic polypeptide”
METDTLLLWVLLLWVPGAHAHHHHHHHHENLYFQSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCSGGGGSGGGGSRELRLESIKSQILSKLRLKEAPNISREVVKQLLPKAPPLQQIL

SEQ ID NO: 11 - protein, 203 aa, Artificial Sequence
  source: /note=“Description of Artificial Sequence: Synthetic polypeptide”
METDTLLLWVLLLWVPGAHAHHHHHHHHENLYFQSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCSGGGGSGGGGSGGGGSSRELRLESIKSQILSKLRLKEAPNISREVVKQLLPKAPPLQQIL

SEQ ID NO: 12 - protein, 214 aa, Artificial Sequence
  source: /note=“Description of Artificial Sequence: Synthetic polypeptide”
METDTLLLWVLLLWVPGAHAHHHHHHHHENLYFQSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCSGGGGSGGGGSGGGGSDGCPVCVWRQHSRELRLESIKSQILSKLRLKEAPNISREVVKQLLPKAPPLQQIL

SEQ ID NO: 13 - protein, 204 aa, Artificial Sequence
  source: /note=“Description of Artificial Sequence: Synthetic polypeptide”
METDTLLLWVLLLWVPGAHAHHHHHHHHENLYFQSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCSGGGGSGGGGSGGGGSSPRELRLESIKSQILSKLRLKEAPNISREVVKQLLPKAPPLQQIL

SEQ ID NO: 14 - protein, 217 aa, Artificial Sequence
  source: /note=“Description of Artificial Sequence: Synthetic polypeptide”
METDTLLLWVLLLWVPGAHAHHHHHHHHENLYFQSDGCPVCVWRQHSRELRLESIKSQILSKLRLKEAPNISREVVKQLLPKAPPLQQILDLHDFQGDGGGGSGGGGSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 15 - protein, 211 aa, Artificial Sequence
  source: /note=“Description of Artificial Sequence: Synthetic polypeptide”
METDTLLLWVLLLWVPGAHAHHHHHHHHENLYFQSNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCSGGGGSGGGGSGGGGSSRELRLESIKSQILSKLRLKEAPNISREVVKQLLPKAPPLQQILDLHDFQGD

SEQ ID NO: 16 - DNA, 636 bp, Artificial Sequence
  source: /note=“Description of Artificial Sequence: Synthetic polynucleotide”
atggagacagacacactcctgctgtgggtactgctgctctgggttccaggagctcacgctcatcatcatcaccatcaccatcatgagaacctgtacttccagagccgagagctgagacttgagagcatcaagtctcagatcctgagcaaactgcggctcaaggaggctcccaacatcagtcgcgaggtggtgaagcagttgctgcccaaggctcctccactgcaacagatcttggacctacacgacttccagggtgacgctggaggtggaggttctggcggtggaggatccggtggaggtggatctaacctgggtctggactgcgacgagcactcaagcgagtcccgctgctgtaggtatcctctcacagtggactttgaggctttcggctgggactggatcatcgcacctaagcgctacaaggccaactactgctccggccagtgcgagtacatgttcatgcagaagtacccccatacccatttggtgcagcaggccaatccaagaggctctgctggaccctgttgtacccctaccaagatgtccccaatcaacatgctctacttcaacgacaagcagcagatcatctacggcaagatccctggcatggtggtggatcgctgtggctgctcttga

SEQ ID NO: 17 - DNA, 654 bp, Artificial Sequence
  source: /note=“Description of Artificial Sequence: Synthetic polynucleotide”
atggagacagacacactcctgctgtgggtactgctgctctgggttccaggagctcacgctcatcatcatcaccatcaccatcatgagaacctgtacttccagagcgatggttgccctgtgtgcgtttggcgacagcacagccgagagctgagacttgagagcatcaagtctcagatcctgagcaaactgcggctcaaggaggctcccaacatcagtcgcgaggtggtgaagcagttgctgcccaaggctcctccactgcaacagatcttggacctacacgacttccagggtgacggcggtggaggatccggtggaggtggatctaacctgggtctggactgcgacgagcactcaagcgagtcccgctgctgtaggtatcctctcacagtggactttgaggctttcggctgggactggatcatcgcacctaagcgctacaaggccaactactgctccggccagtgcgagtacatgttcatgcagaagtacccccatacccatttggtgcagcaggccaatccaagaggctctgctggaccctgttgtacccctaccaagatgtccccaatcaacatgctctacttcaacgacaagcagcagatcatctacggcaagatccctggcatggtggtggatcgctgtggctgctcttga

SEQ ID NO: 18 - protein, 5 aa, Artificial Sequence
  source: /note=“Description of Artificial Sequence: Synthetic peptide”
KRSRR

SEQ ID NO: 19 - protein, 15 aa, Artificial Sequence
  source: /note=“Description of Artificial Sequence: Synthetic peptide”
GGGGSGGGGSGGGGS

SEQ ID NO: 20 - skipped

SEQ ID NO: 21 - protein, 45 aa, Artificial Sequence
  source: /note=“Description of Artificial Sequence: Synthetic polypeptide”
SPRELRLESIKSQILSKLRLKEAPNISREVVKQLLPKAPPLQQIL

SEQ ID NO: 22 - protein, 19 aa, Artificial Sequence
  source: /note=“Description of Artificial Sequence: Synthetic peptide”
SRELRLESIKSQILSKLRL

SEQ ID NO: 23 - protein, 16 aa, Homo sapiens
APNISREVVKQLLPKA

SEQ ID NO: 24 - protein, 11 aa, Artificial Sequence
  source: /note=“Description of Artificial Sequence: Synthetic peptide”
DGCPVCVWRQH

SEQ ID NO: 25 - protein, 9 aa, Homo sapiens
ISREVVKQL

SEQ ID NO: 26 - protein, 405 aa, Mus musculus
MVLAAPLLLGFLLLALELRPRGEAAEGPAAAAAAAAAAAGVGGERSSRPAPSAPPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 27 - protein, 390 aa, Danio rerio
MKRYNFLLCLTVLISLGLSGSDEPNLFLAPLSEMSSDIGVSLFDVDDVESSECSACVWREQSKVLRLETIKSQILSKLRLKQAPNISREVVNQLLPKAPPLQQLLDHHDFQGDASSLEDFIDADEYHATTESVITMASEPEPLVQVDGKPTCCFFKFSPKLMFTKVLKAQLWVYLQPLKQTSTVYLQILRLKPITEQGSRHIRIRSLKIELDSQAGHWQSIDFKHVLQNWFKQPHTNWGIDINAYDESGNDLAVTSLGPGEEGLQPFLEVKILETTKRSRRNLGLDCDEHSTESRCCRYPLTVDFEAFGWDWIIAPKRYKANYCSGQCEYMFMQKYPHTHLVQHANPRGSAGPCCTPTKMSPINMLYFNDKQQIIHGKIPGMVVDRCGCS

SEQ ID NO: 28 - protein, 40 aa, Artificial Sequence
  source: /note=“Description of Artificial Sequence: Synthetic polypeptide”
SPRELRLESIKSQILSKLRLKEAPNISREVVKQLLPKAPP

SEQ ID NO: 29 - protein, 27 aa, Artificial Sequence
  source: /note=“Description of Artificial Sequence: Synthetic peptide”
SPRELRLESIKSQILSKLRLKEAPNIS

SEQ ID NO: 30 - protein, 37 aa, Artificial Sequence
  source: /note=“Description of Artificial Sequence: Synthetic polypeptide”
DGCPVCVWRQHSRELRLESIKSQILSKLRLKEAPNIS

SEQ ID NO: 31 - protein, 32 aa, Artificial Sequence
  source: /note=“Description of Artificial Sequence: Synthetic polypeptide”
DGCPVCVWRQHSRELRLESIKSQILSKLRLKG

SEQ ID NO: 32 - protein, 22 aa, Artificial Sequence
  source: /note=“Description of Artificial Sequence: Synthetic peptide”
SPRELRLESIKSQILSKLRLKG

SEQ ID NO: 33 - protein, 7 aa, Artificial Sequence
  source: /note=“Description of Artificial Sequence: Synthetic peptide”
REVVKQL

SEQ ID NO: 34 - protein, 4 aa, Artificial Sequence
  source: /note=“Description of Artificial Sequence: Synthetic peptide”
GGGS

SEQ ID NO: 35 - protein, 4 aa, Artificial Sequence
  source: /note=“Description of Artificial Sequence: Synthetic peptide”
SGGG

SEQ ID NO: 36 - protein, 5 aa, Artificial Sequence
  source: /note=“Description of Artificial Sequence: Synthetic peptide”
  source: /note=“See specification as filed for detailed description of substitutions and preferred embodiments”
SGGGG

SEQ ID NO: 37 - protein, 6 aa, Artificial Sequence
  source: /note=“Description of Artificial Sequence: Synthetic peptide”
GGGGGS

SEQ ID NO: 38 - protein, 6 aa, Artificial Sequence
  source: /note=“Description of Artificial Sequence: Synthetic peptide”
SGGGGG

SEQ ID NO: 39 - protein, 7 aa, Artificial Sequence
  source: /note=“Description of Artificial Sequence: Synthetic peptide”
GGGGGGS

SEQ ID NO: 40 - protein, 7 aa, Artificial Sequence
  source: /note=“Description of Artificial Sequence: Synthetic peptide”
SGGGGGG

SEQ ID NO: 41 - protein, 25 aa, Artificial Sequence
  source: /note=“Description of Artificial Sequence: Synthetic peptide”
  MOD_RES (4)..(5): Any amino acid
  MOD_RES (9)..(10): Any amino acid
  MOD_RES (14)..(15): Any amino acid
  MOD_RES (19)..(20): Any amino acid
  MISC_FEATURE (1)..(20): /note=“This region may encompass 0-4 ′Gly Gly Gly Xaa Xaa′ repeating units”
  source: /note=“See specification as filed for detailed description of substitutions and preferred embodiments”
GGGXXGGGXXGGGXXGGGXXGGGGS

SEQ ID NO: 42 - protein, 25 aa, Artificial Sequence
  source: /note=“Description of Artificial Sequence: Synthetic peptide”
  MOD_RES (6)..(6): Any amino acid
  MOD_RES (11)..(11): Any amino acid
  MOD_RES (16)..(16): Any amino acid
  MOD_RES (21)..(21): Any amino acid
  MISC_FEATURE (6)..(25): /note=“This region may encompass 0-4 ′Xaa Gly Gly Gly Ser′ repeating units”
  source: /note=“See specification as filed for detailed description of substitutions and preferred embodiments”
GGGGSXGGGSXGGGSXGGGSXGGGS

SEQ ID NO: 43 - protein, 25 aa, Artificial Sequence
  source: /note=“Description of Artificial Sequence: Synthetic peptide”
  MISC_FEATURE (1)..(20): /note=“This region may encompass 0-4 ′Gly Gly Gly Pro Ser′ repeating units”
GGGPSGGGPSGGGPSGGGPSGGGGS

SEQ ID NO: 44 - protein, 25 aa, Artificial Sequence
  source: /note=“Description of Artificial Sequence: Synthetic peptide”
  MISC_FEATURE (1)..(20): /note=“This region may encompass 0-4 ′Gly Gly Gly Gly Gln′ repeating units”
GGGGQGGGGQGGGGQGGGGQGGGGS

SEQ ID NO: 45 - protein, 25 aa, Artificial Sequence
  source: /note=“Description of Artificial Sequence: Synthetic peptide”
  MISC_FEATURE (1)..(20): /note=“This region may encompass 0-4 ′Gly Gly Gly Gly Ala′ repeating units”
GGGGAGGGGAGGGGAGGGGAGGGGS

SEQ ID NO: 46 - protein, 25 aa, Artificial Sequence
  source: /note=“Description of Artificial Sequence: Synthetic peptide”
  MISC_FEATURE (6)..(25): /note=“This region may encompass 0-4 ′Pro Gly Gly Gly Ser′ repeating units”
GGGGSPGGGSPGGGSPGGGSPGGGS

SEQ ID NO: 47 - protein, 15 aa, Artificial Sequence
  source: /note=“Description of Artificial Sequence: Synthetic peptide”
GGGGAGGGGAGGGGS

SEQ ID NO: 48 - protein, 15 aa, Artificial Sequence
  source: /note=“Description of Artificial Sequence: Synthetic peptide”
GGGGQGGGGQGGGGS

SEQ ID NO: 49 - protein, 15 aa, Artificial Sequence
  source: /note=“Description of Artificial Sequence: Synthetic peptide”
GGGPSGGGPSGGGGS

SEQ ID NO: 50 - protein, 15 aa, Artificial Sequence
  source: /note=“Description of Artificial Sequence: Synthetic peptide”
GGGGSPGGGSPGGGS

SEQ ID NO: 51 - protein, 20 aa, Artificial Sequence
  source: /note=“Description of Artificial Sequence: Synthetic peptide”
RELRLESIKSQILSKLRLKG

SEQ ID NO: 52 - protein, 45 aa, Artificial Sequence
  source: /note=“Description of Artificial Sequence: Synthetic polypeptide”
  MOD_RES (7)..(7): Any non-natural amino acid
  MOD_RES (11)..(11): Any non-natural amino acid
  source: /note=“See specification as filed for detailed description of substitutions and preferred embodiments”
SPRELRXESIXSQILSKLRLKEAPNISREVVKQLLPKAPPLQQIL

SEQ ID NO: 53 - protein, 45 aa, Artificial Sequence
  source: /note=“Description of Artificial Sequence: Synthetic polypeptide”
  MOD_RES (8)..(8): Any non-natural amino acid
  MOD_RES (12)..(12): Any non-natural amino acid
  source: /note=“See specification as filed for detailed description of substitutions and preferred embodiments”
SPRELRLXSIKXQILSKLRLKEAPNISREVVKQLLPKAPPLQQIL

SEQ ID NO: 54 - protein, 45 aa, Artificial Sequence
  source: /note=“Description of Artificial Sequence: Synthetic polypeptide”
  MOD_RES (21)..(21): Any non-natural amino acid
  MOD_RES (25)..(25): Any non-natural amino acid
  source: /note=“See specification as filed for detailed description of substitutions and preferred embodiments”
SPRELRLESIKSQILSKLRLXEAPXISREVVKQLLPKAPPLQQIL

SEQ ID NO: 55 - protein, 45 aa, Artificial Sequence
  source: /note=“Description of Artificial Sequence: Synthetic polypeptide”
  MOD_RES (15)..(15): Any non-natural amino acid
  MOD_RES (22)..(22): Any non-natural amino acid
  source: /note=“See specification as filed for detailed description of substitutions and preferred embodiments”
SPRELRLESIKSQIXSKLRLKXAPNISREVVKQLLPKAPPLQQIL

SEQ ID NO: 56 - protein, 45 aa, Artificial Sequence
  source: /note=“Description of Artificial Sequence: Synthetic polypeptide”
  MOD_RES (28)..(28): Any non-natural amino acid
  MOD_RES (32)..(32): Any non-natural amino acid
  source: /note=“See specification as filed for detailed description of substitutions and preferred embodiments”
SPRELRLESIKSQILSKLRLKEAPNISXEVVXQLLPKAPPLQQIL

SEQ ID NO: 57 - protein, 45 aa, Artificial Sequence
  source: /note=“Description of Artificial Sequence: Synthetic polypeptide”
  MOD_RES (33)..(33): Any non-natural amino acid
  MOD_RES (40)..(40): Any non-natural amino acid
  source: /note=“See specification as filed for detailed description of substitutions and preferred embodiments”
SPRELRLESIKSQILSKLRLKEAPNISREVVKXLLPKAPXLQQIL

SEQ ID NO: 58 - protein, 407 aa, Pan troglodytes; identical to SEQ ID NO: 1

SEQ ID NO: 59 - protein, 376 aa, Bos taurus
  MOD_RES (1)..(1): Any amino acid
XAAAAAAAAAGAGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIDLHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 60 - protein, 405 aa, Rattus norvegicus
MVLAAPLLLGFLLLALELRPRGEAAEGPAAAAAAAAAAAGVGGERSSRPAPSAAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 61 - protein, 67 aa, Sus scrofa
LSTCKTIDMELVKRKRIEAIRGQILSKLRLASPPSQGDVPPGPLPEAVLALYNSTRDRVAGESVEPE

SEQ ID NO: 62 - protein, 73 aa, Homo sapiens
DGCPVCVWRQHSRELRLESIKSQILSKLRLKEAPNISREVVKQLLPKAPPLQQILDLHDFQGDALQPEDFLEE

SEQ ID NO: 63 - protein, 76 aa, Sus scrofa
LSTCKTIDMELVKRKRIEAIRGQILSKLRLASPPSQGDVPPGPLPEAVLALYNSTRDRVAGESVEPEPEPEADYYA

SEQ ID NO: 64 - protein, 55 aa, Homo sapiens
DGCPVCVWRQHSRELRLESIKSQILSKLRLKEAPNISREVVKQLLPKAPPLQQIL

SEQ ID NO: 65 - protein, 108 aa, Homo sapiens
AEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEE

SEQ ID NO: 66 - protein, 78 aa, Sus scrofa
LSTCKTIDMELVKRKRIEAIRGQILSKLRLASPPSQGDVPPGPLPEAVLALYNSTRDRVAGESVEPEPEPEADYYAKE

SEQ ID NO: 67 - protein, 139 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATT

SEQ ID NO: 68 - protein, 103 aa, Homo sapiens
DFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADM

SEQ ID NO: 69 - protein, 9 aa, Mycoplasma pneumoniae
ISSEVVKQL

SEQ ID NO: 70 - protein, 11 aa, Homo sapiens
ISREVVKQLLP

SEQ ID NO: 71 - protein, 11 aa, Methanothermobacter thermautotrophicus
ITRAVSKQLLP

SEQ ID NO: 72 - protein, 10 aa, Homo sapiens
ISREVVKQLL

SEQ ID NO: 73 - protein, 10 aa, Bacillus subtilis
ISREMMKELL

SEQ ID NO: 74 - protein, 11 aa, Escherichia coli
ITRGVSKQLLP

SEQ ID NO: 75 - protein, 7 aa, Homo sapiens
PNISREV

SEQ ID NO: 76 - protein, 7 aa, Pyrococcus horikoshii
PNISREI

SEQ ID NO: 77 - protein, 7 aa, Staphylococcus aureus
REIVKQL

SEQ ID NO: 78 - protein, 12 aa, Homo sapiens
APNISREVVKQL

SEQ ID NO: 79 - protein, 12 aa, Burkholderia cenocepacia
APNDSRDVASQL

SEQ ID NO: 80 - protein, 15 aa, Homo sapiens
APNISREVVKQLLPK

SEQ ID NO: 81 - protein, 15 aa, Bacillus subtilis
AAKVSREKVPDLLPK

SEQ ID NO: 82 - protein, 8 aa, Homo sapiens
REVVKQLL

SEQ ID NO: 83 - protein, 8 aa, Lactobacillus plantarum
RDVVRQLL

SEQ ID NO: 84 - protein, 6 aa, Homo sapiens
VKQLLP

SEQ ID NO: 85 - protein, 6 aa, Sus scrofa
VKQLLP

SEQ ID NO: 86 - protein, 8 aa, Homo sapiens
PNISREVV

SEQ ID NO: 87 - protein, 8 aa, Medicago truncatula
PNTSRDVV

SEQ ID NO: 88 - protein, 6 aa, Homo sapiens
EVVKQL

SEQ ID NO: 89 - protein, 6 aa, Staphylococcus epidermidis
EVVKQL